(19) United States
(12) Patent Application Publication
(10) Pub. No.: US 2023/0014092 A1
(43) Pub. Date: Jan. 19, 2023

(54) MATERIALS AND METHODS FOR MONITORING INFLAMMATION

(71) Applicant: Janssen Biotech, Inc., Horsham, PA (US)

(72) Inventors: Ernesto MUNOZ, Horsham, PA (US); Julianty ANGSANA, Horsham, PA (US); Carl MANTHEY, Horsham, PA (US); Xuejun LIU, Horsham, PA (US)

(21) Appl. No.: 17/757,199

(22) PCT Filed: Dec. 11, 2020
(86) PCT No.: PCT/US2020/064631

§ 371 Date: Jun. 10, 2022

Related U.S. Application Data
(60) Provisional application No. 62/948,131, filed on Dec. 13, 2019.
(60) Provisional application No. 63/055,661, filed on Jul. 23, 2020.

(51) Int. Cl. G01N 33/50

(52) U.S. Cl. CPC G01N 33/5023; G01N 2333/545; G01N 2800/52

(57) ABSTRACT

A method of identifying a subject who is likely to be responsive to a treatment comprising an inhibitor of IL1α, or predicting the responsiveness of a subject to a treatment comprising an inhibitor of IL1α, comprising providing a sample from a subject; administering the inhibitor of IL1α to the sample; measuring levels of one or more biomarkers in the sample; and determining the pharmacodynamic or the pharmacokinetic effect of the inhibitor of IL1α based on the levels of the one or more biomarkers.

### CROSS-REFERENCE TO RELATED APPLICATIONS

The present application claims the priority of U.S. provisional patent application Ser. No. 62/948,131 filed on Dec. 13, 2019, and U.S. provisional patent application Ser. No. 63/055,661 filed on Jul. 23, 2020.

### 1. FIELD

Provided herein, in some embodiments, are methods of using certain biomarkers in assessing and monitoring a pharmacodynamics or pharmacokinetic effect of an inhibitor of Interleukin 1 alpha (IL1α), and in predicting and monitoring clinical sensitivity and therapeutic response to an inhibitor of IL1α, and kits for performing same. Also provided herein, in certain embodiments, are methods of screening or identifying an inhibitor of IL1α.

### 2. BACKGROUND

Inflammation is a common pathogenesis of many diseases, including cardiovascular and bowel diseases, diabetes, arthritis, and cancer. Inflammation is a biological response of the immune system that can be triggered by a variety of factors, including pathogens, damaged cells and toxic compounds. These factors may induce acute and/or chronic inflammatory responses in the heart, pancreas, liver, kidney, lung, brain, intestinal tract and reproductive system, potentially leading to tissue damage or disease. Chen et al., Oncotarget. 9(6): 7204-7218 (2018).

In response to tissue injury, the body initiates a chemical signaling cascade that stimulates responses aimed at healing affected tissues. These signals activate leukocyte chemotaxis from the general circulation to sites of damage. Id.

IL1α is a cytokine of the interleukin 1 family that is encoded by the IL1A gene in humans. In general, Interleukin 1 is responsible for the production of inflammation. IL1α inhibitors are being developed to interrupt those processes and treat diseases. Blocking the activity of IL1α has the potential to treat various diseases or conditions including skin diseases such as acne. See, e.g., Valente Duarte de Sousa, Expert Opinion on Investigational Drugs. 23 (10): 1389-410 (2014).

There is a need in the art to assess effects (including a pharmacodynamics or pharmacokinetic effect) of IL1α inhibitors as well as identifying additional IL1α inhibitors for therapeutic or other uses.

### 3. SUMMARY OF THE INVENTION

In one aspect, provided herein is a method of identifying a subject who is likely to be responsive to a treatment comprising an inhibitor of IL1a, or predicting the responsiveness of a subject to a treatment comprising an inhibitor of IL1α, comprising:
i. providing a sample from the subject;
ii. administering the inhibitor of IL1α to the sample;
iii. measuring levels of one or more biomarkers in the sample;
iv. identifying the subject as being likely to be responsive to the treatment comprising the inhibitor of IL1α, or predicting the responsiveness of the subject to the treatment comprising the inhibitor of IL1α, based on the levels of the one or more biomarkers as measured in step (iii).

In another aspect, provided herein is a method of selectively treating a subject with a treatment comprising an inhibitor of IL1α, comprising administering a therapeutically effective amount of the treatment comprising the inhibitor of IL1α to the subject identified as being likely to be responsive to the treatment comprising the inhibitor of IL1α according to the method provided herein.

In some embodiments, the sample comprises a skin cell. In some embodiments, the sample comprises an injured skin cell.

In some embodiments, the sample is obtained by a skin biopsy procedure. In some embodiments, the size of the sample is about 3.5 mm to 4.5 mm.

In some embodiments, the method further comprises culturing the sample ex vivo prior to administering the inhibitor of IL1α to the sample. In other embodiments, the method further comprises culturing the sample ex vivo after administering the inhibitor of IL1α to the sample. In yet other embodiments, the inhibitor of IL1α is administered to the sample while culturing the sample ex vivo.

In some embodiments, the levels of the one or more biomarkers are measured at least 4 hours, at least 5 hours, at least 10 hours, at least 15 hours, at least 20 hours, at least 21 hours, at least 22 hours, at least 23 hours, or at least 24 hours post the administration of the inhibitor of IL1α and/or post the sample is obtained from the subject. In some embodiments, the level of the one or more biomarkers are measured at about 24 hours post the administration of the inhibitor of IL1α and/or post the sample is obtained from the subject.

In some embodiments, the method further comprises comparing the levels of the one or more biomarkers with reference levels of the one or more biomarkers. In some embodiments, the reference levels of the one or more biomarkers are the levels of the one or more biomarkers in a reference sample from the subject prior to administration of the inhibitor of IL1α. In other embodiments, the reference levels of the one or more biomarkers are the levels of the one or more biomarkers in a reference sample from the subject without administration of the inhibitor of IL1α. In yet other embodiments, the reference levels of the one or more biomarkers are pre-determined levels of the one or more biomarkers. In other embodiments, the reference levels of the one or more biomarkers are the levels of the one or more biomarkers in a reference sample administered with a control agent. In some embodiments, the control agent is a positive control agent that inhibits IL1α. In other embodiments, the control agent is a negative control agent that does not inhibit IL1α.

In some embodiments, the lower levels of the one or more biomarkers as compared with reference levels of the one or more biomarkers indicates the subject is likely to be responsive to the treatment comprising the inhibitor of IL1α, or wherein the subject is identified likely to be responsive to the treatment comprising the inhibitor of IL1α if the levels of the one or more biomarkers in the sample are at least 30%, at least 35%, at least 40%, or at least 50% less than the reference levels.

In some embodiments, the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one ore more biomarkers comprise CXCL1. In another embodiment, the one or more biomarkers comprise IL6.

In some embodiments, the one or more biomarkers are GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score.

In some embodiments, the one or more biomarkers are GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score. In some embodiments, the one or more biomarkers are CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. In some embodiments, a composite score is calculated based on the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3, and wherein the method further comprises comparing the composite score to a reference score.

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In one embodiment, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score.

In some embodiments, the levels of the one or more biomarkers are determined by measuring the nucleic acid levels (for example, mRNA) of the one or more biomarkers. In other embodiments, the levels of the one or more biomarkers are determined by measuring the protein levels of the one or more biomarkers.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the levels of the one or more biomarkers are the levels of cytokines secreted by the sample selected from a group consisting of GCSF, CXCL1, IL6 and IL8.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In one embodiment, the one or more biomarkers comprise CSF3 (GCSF). In another embodiment, the one or more biomarkers comprise CXCL1. In another embodiment, the one or more biomarkers comprise IL6.

In some embodiments, the one or more biomarkers are GCSF, CXCL1, IL4, IL6, IL8, MDC, IP10, GMCSF, MIP1a and TGFa. In some embodiments, a composite score is calculated based on the levels of GCSF, CXCL1, IL4, IL6, IL8, MDC, IP10, GMCSF, MIP1a and TGFa, and wherein the method further comprises comparing the composite score to a reference score.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In one embodiment, the one or more biomarkers are CSF3 (GCSF), CXCL1 and IL6. In one embodiment, a composite score is calculated based on the levels of CSF3 (GCSF), CXCL1 and IL6, and wherein the method further comprises comparing the composite score to a reference score.

In some embodiments, the levels of the one or more biomarkers are determined by measuring the protein levels of the one or more biomarkers.

In another aspect, provided herein is a method of identifying a subject having a IL1α mediated disease who is likely to be responsive to a treatment comprising an inhibitor of IL1α or predicting the responsiveness of a subject having a IL1α mediated disease to a treatment comprising an inhibitor of IL1α, comprising:
i. providing a sample from the subject;
ii. measuring levels of one or more biomarkers in the sample;
iii. identifying the subject with the IL1α mediated disease as responsive to the treatment with the inhibitor of IL1α when the levels of one or more biomarkers in the sample are higher than the reference levels of one or more biomarkers; and/or identifying the subject with the IL1α mediated disease as non-responsive to the treatment with the inhibitor of IL1α when the levels of one or more biomarkers in the sample are not higher than the reference levels of one or more biomarkers.

In yet another aspect, provided herein is a method of selectively treating a subject having a IL1α mediated disease with a treatment comprising an inhibitor of IL1α, comprising administering a therapeutically effective amount of the treatment comprising the inhibitor of IL1α to the subject identified responsive to the treatment comprising the inhibitor of IL1αaccording to the method provided herein.

In some embodiments, the sample comprises a skin cell, and wherein optionally the skin cell is an injured skin cell.

In some embodiments, the sample is obtained by a skin biopsy procedure.

In some embodiments, the sample is a blood sample.

In some embodiments, the reference levels of the one or more biomarkers are pre-determined levels of the one or more biomarkers. In other embodiments, the reference level of the one or more biomarkers are the levels of the one or more biomarkers in a sample comprising an uninjured skin cell.

In some embodiments, the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one ore more biomarkers comprise CXCL1. In another embodiment, the one or more biomarkers comprise IL6.

In some embodiments, the one or more biomarkers are GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, IL8, IL4, MDC, IP10, GMCSF (CSF2), MIP1a, TGFa, CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score.

In some embodiments, the one or more biomarkers are GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score.

In some embodiments, the one or more biomarkers are CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. In some embodiments, a composite score is calculated based on the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3, and wherein the method further comprises comparing the composite score to a reference score.

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In one embodiment, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score.

In some embodiments, the levels of the one or more biomarkers are determined by measuring the nucleic acid levels (for example, mRNA) of the one or more biomarkers. In other embodiments, the levels of the one or more biomarkers are determined by measuring the protein levels of the one or more biomarkers.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the levels of the one or more biomarkers are the levels of cytokines secreted by the sample selected from a group consisting of GCSF, CXCL1, IL6 and IL8.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In some embodiments, the one or more biomarkers are GCSF, CXCL1, IL4, IL6, IL8, MDC, IP10, GMCSF, MIP1a and TGFa. In some embodiments, a composite score is calculated based on the levels of GCSF, CXCL1, IL4, IL6, IL8, MDC, IP10, GMCSF, MIP1a and TGFa, and wherein the method further comprises comparing the composite score to a reference score.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In one embodiment, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score.

In some embodiments, the levels of the one or more biomarkers are determined by measuring the protein levels of the one or more biomarkers.

In yet another aspect, provided herein is a method of predicting the responsiveness of a subject having an IL1α mediated disease to an IL1α inhibitor or determining an effective dose of an IL1α inhibitor to be administered to a subject having an IL1α mediated disease comprising:
i. providing a first sample from a subject;
ii. administering an IL1α inhibitor to the subject at a predefined dose;
iii. providing a second sample from the subject;
iv. measuring levels of one or more biomarkers in the first sample and the second sample; and
v. determining that the subject is likely to be responsive to the IL1α inhibitor or the predefined dose is effective when the levels of the one or more biomarkers in the second sample are lower than the levels of one or more biomarkers in the first sample.

In some embodiments, the second sample is obtained at 1.5 to 2 hours post the administration of the IL1α inhibitor.

In some embodiments, the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one ore more biomarkers comprise CXCL1. In another embodiment, the one or more biomarkers comprise IL6.

In some embodiments, the one or more biomarkers are GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, IL8, IL4, MDC, IP10, GMCSF (CSF2), MIP1a, TGFa, CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score.

In some embodiments, the one or more biomarkers are GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1. In some embodiments, a composite score is calculated based on the levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1, and wherein the method further comprises comparing the composite score to a reference score. In some embodiments, the one or more biomarkers are CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. In some embodiments, a composite score is calculated based on the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3, and wherein the method further comprises comparing the composite score to a reference score.

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In one embodiment, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score. In some embodiments, the levels of the one or more biomarkers are determined by measuring the nucleic acid levels (for example, mRNA) of the one or more biomarkers. In other embodiments, the levels of the one or more biomarkers are determined by measuring the protein levels of the one or more biomarkers.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the levels of the one or more biomarkers are the levels of cytokines secreted by the sample selected from a group consisting of GCSF, CXCL1, IL6 and IL8.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In some embodiments, the one or more biomarkers are GCSF, CXCL1, IL4, IL6, IL8, MDC, IP10, GMCSF, MIP1a and TGFa. In some embodiments, a composite score is calculated based on the levels of GCSF, CXCL1, IL4, IL6, IL8, MDC, IP10, GMCSF, MIP1a and TGFa, and wherein the method further comprises comparing the composite score to a reference score.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In one embodiment, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score.

In some embodiments, the levels of the one or more biomarkers are determined by measuring the protein levels of the one or more biomarkers.

In yet another aspect, provided herein is a kit for identifying a subject who is likely to be responsive to a treatment comprising an inhibitor of IL1a, predicting the responsiveness of a subject to a treatment comprising an inhibitor of IL1a, or monitoring the response of a subject to a treatment comprising an inhibitor of IL1α, comprising: (a) an agent for measuring levels of one or more biomarkers in a sample, wherein the one or more biomarkers are selected from (i) a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3, or (ii) a group consisting of GCSF, CXCL1, IL4, IL6, IL8, MDC, IP10, GMCSF, MIP1a and TGFa, or (iii) a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1 and (b) a positive control agent that inhibits IL1α and/or a negative control agent that does not inhibit IL1α.

In some embodiments of the kit, the one or more biomarkers comprise CSF3 (GCSF), CXCL1 and IL6.

In some embodiments of the kit, the one or more biomarkers are GCSF, CXCL1, IL6 and IL8.

In some embodiments of the kit, the one or more biomarkers are CXCL1, GCSF, GMCSF, IL6, IL8, MIP1a and TGFa.

In some embodiments of the kit, the one or more biomarkers are GCSF, CXCL1, IL4, IL6, IL8, MDC and IP10.

In some embodiments of the kit, the one or more biomarkers are GCSF, CXCL1, IL4, IL6, IL8, MDC, IP10, GMCSF, MIP1a and TGFa.

In other embodiments of the kit, the one or more biomarkers are CCL20, CSF3(GCSF), CXCL1, CXCL2, CXCL3, CXCL5, IL6, IL8, and PTGS2.

In other embodiments of the kit, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments of the kit, the one or more biomarkers are CCL20, CCL22, CSF3(GCSF), CXCL1, CXCL2, CXCL3, CXCL5, CXCL6, IL6, IL8, and PTGS2.

In some embodiments of the kit, the one or more biomarkers are CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments of the kit, the one or more biomarkers are GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, G0S2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment of the kit, the one or more biomarkers are GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the kit further comprises a tool for obtaining the sample from a subject. In other embodiments, the tool is suitable for obtaining a skin sample from the subject. In other embodiments, the kit further comprises a tool for administering the inhibitor of IL1α to the sample.

In some embodiments, the positive control agent is an antibody that binds to IL1α.

In some embodiments of the various methods and kits provided herein, the subject has atopic dermatitis, hidradenitis suppurativa, psoriasis, cutaneous lupus erythematosus, or autoimmune bullous disease. In one embodiment, the subject has atopic dermatitis. In another embodiment, the subject has hidradenitis suppurativa.

### 4. BRIEF DESCRIPTION OF THE FIGURES

FIGS. 1A and 1B show the level of IL1α is elevated in ex vivo biopsy explant culture model at both 4-hour and 24-hour time point. FIG. 1A illustrates the ex vivo biopsy explant culture experimental setup. FIG. 1B shows that secreted IL1α level was measured using Luminex and was shown to be upregulated both at 4 hr and 24 hr time point compared to control (n=4 mm biopsy, 5-8 donors).

FIGS. 2A and 2B show the effects of IL1α blockade on secreted cytokine levels in supernatant of skin biopsy explant culture at 4 hr (3 mm, 4 mm or 6 mm). FIG. 2A illustrates the ex vivo biopsy explant culture experimental setup to evaluate PD effects of IL1α blockade. FIG. 2B shows the level of secreted cytokine was measured using Luminex (Millipore, HCYTMAG-60K-PX41) after 4 hours of ex vivo biopsy explant culture using 3 mm, 4 mm or 6 mm skin biopsies (1 donor, 3 replicates per donor). Treatment using anti-IL1α(open round dot) significantly reduced cytokine level compared to samples cultured with no treatment (black round dot) for all skin biopsy size evaluated. However, dynamic range (difference between 4 hr and 4 hr+anti-IL1α) was much smaller in 3 mm than 4 mm or 6 mm biopsy size.

FIG. 3 shows lidocaine injection affects ability of anti-IL1α to reduce level of GCSF, CXCL1 and IL8 in culture supernatant at 4-hour, but not at 24-hour time point. The level of secreted cytokine was measured using Luminex after 4 hours or 24 hours of ex vivo biopsy explant culture using biopsies (4 mm) that were obtained from skin that were injected with PBS (black circle) or 1% lidocaine (black square), (n=5 donors for 4-hour, n=3 donors for 24-hour, each dot or square represent the average of 3-5 replicates from each donor).

FIGS. 4A and 4B show that anti-IL1α treatment can reduce elevated level of GCSF, CXCL1, IL4, IL6, IL8, MDC and IP10 cytokines in supernatant of ex vivo biopsy explant culture model (with lidocaine injection) at 24-hour time point. The level of secreted cytokine was measured using Luminex after 24 hours of ex vivo biopsy explant culture using biopsies (4 mm) that were obtained from skin that were injected with 1% lidocaine (3 donors, 1-5 replicates per donor).

FIGS. 4C to 4F show the evaluation of the ex vivo biopsy explant culture model in 10 healthy volunteers. FIG. 4C shows that luminex analysis of the culture supernatant showed the induction of various cytokines following 24 hour of culture. FIG. 4D shows that treatment with anti-IL1α can reduce the level of a subset of cytokines, i.e., CXCL1, GCSF, GMCSF, IL8, IL8, MIP1a and TGFa. The level of these cytokines were reduced by anti-IL1α treatment (>50% inhibition) and display low variance (<100%) in their inhibition response. FIG. 4E shows the induction fold change of these cytokines in the 10 healthy volunteers. FIG. 4F shows the concentrations of these cytokines in the 10 healthy volunteers.

FIGS. 5A and 5B show that anti-IL1α treatment reduces elevated level in gene expression of CCL20, CCL22, CSF3(GCSF), CXCL1, CXCL2, CXCL3, CXCL5, CXCL6, IL6, IL8, and PTGS2 in ex vivo biopsy explant culture model (with lidocaine injection) at 24-hour time point. Gene expression in ex vivo biopsy explant culture model (with lidocaine injection) was measured using Nanostring platform (Nanostring, nCounter Human Inflammation v2 panel). 40 of the 249 tested genes were induced at 24-hour time point, with geometric mean of fold change>2 among skin samples derived from three donors (FIG. 5A). Elevated gene expression in 11 of the 40 induced genes was inhibited by anti-IL1α treatment, with an average of inhibition>30% (FIG. 5B). D1-3: three donors.

FIGS. 5C, 5D, and 5E show anti-IL1α treatment reduces elevated level in gene expression of CCL20, CCL3, CCL4, CCL8, CFB, CSF3(GMCSF), CXCL1, CXCL2, CXCL3, CXCL5, IL1B, IL6, IL8, MMP3, and PTGS2 in ex vivo biopsy explant culture model (with lidocaine injection) at 24-hour time point as measured using Nanostring in 30 skin biopsies from ten donors, with three skin biopsies per donor. FIG. 5C shows that 25 of the 249 tested genes were induced at 24-hour time point, with fold change>2 in skin samples derived from all ten donors. Elevated gene expression in 15 of the 25 cut-induced genes was inhibited by anti-IL1α treatment, with an average of inhibition>30%. FIG. 5D shows induction of the 15 genes (upper panel) and inhibition by anti-IL1α(lower pannel) among ten donors. FIG. 5E shows that elevated gene expression in 15 of the 29 induced genes was inhibited by anti-IL1α treatment, with an average of inhibition>30%. These 15 genes were defined as IL1α signature. Geomean of induced fold change of the 15 genes (upper panel) and the average of % inhibition on induction by anti-IL1α(lower panel) among ten donors are shown.

FIG. 6 shows anti IL-1a treatment can reduce elevated level in gene expression of a subset of injury-induced genes in ex vivo biopsy explant culture model at 24-hour time point. Gene expression in ex vivo biopsy explant culture model was measured using RNAseq. 1287 genes were induced after 24-hour culture (vs control) with geometric average of fold change>2 among the 10 donors, and false discovery rate (FDR)<0.05; all these 1287 genes were also induced (fold change>1.5) in each of the 10 donors. Elevated gene expression in 139 of these 1287 injury-induced genes was inhibited by anti-IL1α treatment, with an average of inhibition>20%.

FIG. 7 shows bermekimab can reduce elevated level of a subset of induced cytokines in ex vivo biopsy explant culture model at 24-hour time point. FIG. 7A indicates that measurement of cytokine levels in the supernatant showed that bermekimab can reduce the level of GCSF, CXCL1, IL6 and IL8 in a dose responsive manner. FIG. 7B indicates that measurement of cytokine levels in the skin tissue lysate showed that bermekimab can reduce the level of CXCL1, IL6 and IL8 in a dose responsive manner.

FIG. 8 is a schematic summarizing the Phase 1 study of Example 7.

### 5. DETAILED DESCRIPTION OF THE INVENTION

The present disclosure provides in part an effective ex vivo skin biopsy-based assay and novel biomarkers that allow for the measurement of various effects of an anti IL1α treatment.

### 5.1. Definitions

As used herein, and unless otherwise specified, the terms “treat,” “treating,” and “treatment” refer to an action that occurs while a patient is suffering from a disease or disorder (e.g., such as a disease or disorder related to inflammation), which reduces the severity of the disease or disorder or retards or slows the progression of the disease or disorder.

The term “responsiveness” or “responsive” when used in reference to a treatment refers to the degree of effectiveness of the treatment in lessening or decreasing the symptoms of a disease or disorder being treated. An improvement in a disease or disorder can be characterized as a complete or partial response. “Complete response” refers to an absence of clinically detectable disease with normalization of any previously abnormal measurements. “Partial response” refers to at least about 10%, about 20%, about 30%, about 40%, about 50%, about 60%, about 70%, about 80%, or about 90% decrease in any measurable parameters of a disease or disorder.

As used herein, and unless otherwise specified, the term “therapeutically effective amount” of a compound is an amount sufficient to provide a therapeutic benefit in the treatment or management of a disease or disorder, or to delay or minimize one or more symptoms associated with the presence of the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of the disease or disorder, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “likelihood” generally refers to an increase in the probability of an event. The term “likelihood” when used in reference to the effectiveness of a patient response can mean the increase of indicators, such as mRNA or protein expression, that may evidence an increase in the progress in treating a disease or disorder.

The term “predict” generally means to determine or tell in advance. When used to “predict” the effectiveness or responsiveness of a treatment, for example, the term “predict” can mean that the likelihood of the outcome of the treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the response of a subject to a treatment” refers to tracking the effectiveness or responsiveness in treating a disease or disorder in a patient. The monitoring can be performed, for example, by following the expression of mRNA or protein biomarkers.

A “biological marker” or “biomarker” is a substance whose detection indicates a particular biological state, such as, for example, the responsiveness to a treatment. In some embodiments, biomarkers can be determined individually. In other embodiments, several biomarkers can be measured simultaneously. In some embodiments, a “biomarker” indicates a change in the level of mRNA expression that may correlate with the progression of a disease, or with the susceptibility of the disease to a given treatment. In some embodiments, the biomarker is a nucleic acid, such as mRNA or cDNA. In additional embodiments, a “biomarker” indicates a change in the level of polypeptide or protein expression that may correlate with the risk or progression of a disease, or patient's susceptibility to treatment. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

The term “expression level” refers to the amount, accumulation, or rate of a biomarker molecule or a gene set. An expression level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “expression level” refers to an absolute amount of a molecule in a sample or a relative amount of the molecule, determined under steady-state or non-steady-state conditions.

An mRNA that is “upregulated” is generally increased upon a given treatment or condition, or in certain patient groups. An mRNA that is “downregulated” generally refers to a decrease in the level of expression of the mRNA in response to a given treatment or condition, or in certain patient groups. In some situations, the mRNA level can remain unchanged upon a given treatment or condition. An mRNA from a patient sample can be “upregulated” when treated with a drug, as compared to a non-treated control. This upregulation can be, for example, an increase of about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control mRNA level. Alternatively, an mRNA can be “downregulated”, or expressed at a lower level, in response to administration of certain compounds or other agents. A downregulated mRNA can be, for example, present at a level of about 99%, about 95%, about 90%, about 80%, about 70%, about 60%, about 50%, about 40%, about 30%, about 20%, about 10%, about 1%, or less of the comparative control mRNA level.

Similarly, the level of a polypeptide or protein biomarker from a patient sample can be increased when treated with a drug, as compared to a non-treated control. This increase can be about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control protein level. Alternatively, the level of a protein biomarker can be decreased in response to administration of certain compounds or other agents. This decrease can be, for example, present at a level of about 99%, about 95%, about 90%, about 80%, about 70%, about 60%, about 50%, about 40%, about 30%, about 20%, about 10%, about 1%, or less of the comparative control protein level.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The terms “isolated” and “purified” refer to isolation of a substance (such as mRNA, DNA, or protein) such that the substance comprises a substantial portion of the sample in which it resides, i.e., greater than the portion of the substance that is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” indicates direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and embodiments where the attachment is indirect.

The term “sample” as used herein relates to a material or mixture of materials, typically, although not necessarily, in a tissue biopsy form, containing one or more components of interest. In some embodiments, the sample is a biological sample, which refers to a sample obtained from a biological subject, including a sample of biological tissue or fluid origin, obtained, reached, or collected in vivo or in situ. A biological sample also includes samples from a region of a biological subject containing injured cells or tissues. Such samples can be, but are not limited to, organs, tissues, and cells isolated from a mammal. Exemplary biological samples include but are not limited to cell lysate, cells, tissues, organs, organelles, a biological fluid, a blood sample, a urine sample, a skin sample, and the like.

The term “inhibit” as used herein in connection with IL1α refers to reducing any biological activity of IL1α, and an “inhibitor of IL1α” refers to an agent that can reduce any biological activity of IL1α. For example, such an inhibitor may partially, substantially or completely inhibit the biological activity of IL1α.

The term “polymerase chain reaction” or “PCR” as used herein generally refers to a procedure wherein small amounts of a nucleic acid, RNA and/or DNA, are amplified as described, for example, in U.S. Pat. No. 4,683,195. Generally, sequence information from the ends or beyond of the region of interest needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 1987, 51:263-273; PCR Technology (Stockton Press, NY, Erlich, ed., 1989).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It should be noted that if there is a discrepancy between a depicted structure and a name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (4th ed. 2014); Glover, ed., DNA Cloning, Volumes I and II (2^{nd} ed. 1995); Immunochemical Methods in Cell and Molecular Biology (Academic Press, London); Scopes, Protein Purification: Principles and Practice (Springer Verlag, N.Y., 3rd ed. 1993); and Weir & Blackwell, eds., Handbook of Experimental Immunology, Volumes I-IV (5^{th} ed. 1996).

Bermekimab is a monoclonal anti-IL-1a antibody having heavy and light chain amino acid sequences of SEQ ID NO: 158 and SEQ ID NO: 159 respectively.

### 5.2. Biomarkers and Methods of Use Thereof

5.2.1. Biomarkers for Pharmacodynamics or Pharmacokinetic Effects

As shown in Section 6 below, elevated levels of gene expression of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 or a subset thereof in injured skin biopsy (e.g., following 24 hours of culture) are reduced by treatment with an IL1α inhibitor (in this case, an anti-IL1α antibody), and thus expression levels of these biomarkers or a subset thereof can be used to evaluate effects of IL1α blockade (in particular, IL1α blockade with an anti-IL1α antibody). Similarly, elevated levels of gene expression of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL5, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, G0S2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1 are reduced by treatment with an IL1α inhibitor (in this case, an anti-IL1α antibody), and thus expression of these biomarkers or a subset thereof can be used to evaluate the effects of IL1α blockade (in particular, IL1α blockade with an anti-IL1α antibody). Similarly, secretion of GCSF, CXCL1, IL4, IL6, IL8, MDC, IP10, GMCSF, MIP1a and TGFa or a subset thereof in injured skin biopsy are reduced by treatment with an IL1α inhibitor (for example, an anti-IL1α antibody, such as bermekimab), and thus these cytokines can also be used to evaluate effects of IL1α blockade (in particular, IL1α blockade with an anti-IL1α antibody, such as bermekimab).

In one aspect, provided herein is a method of determining a pharmacodynamics (PD) or pharmacokinetic (PK) effect of an inhibitor of IL1α comprising providing a sample from a subject; administering the inhibitor of IL1α to the sample; measuring levels of one or more biomarkers in the sample; and determining the pharmacodynamic or the pharmacokinetic effect of the inhibitor of IL1α based on the levels of the one or more biomarkers. In some embodiments, the method is used for determining the PD effect of the inhibitor of IL1α.

In some embodiments, the sample is obtained by biopsy procedure and contain injured skin cells. Typically the injury arises as a result of the biopsy procedure.

In one embodiment, the sample is a skin sample.

The size of a skin sample may affect the measurement of a pharmacodynamic (PD) or pharmacokinetic (PK) effect of an inhibitor of IL1α. For example, as shown in Section 6 below, evaluation of the ex vivo skin explant-based assay at 4-hour time point indicate that 4 mm biopsy size is effective for measurement of PD response. For clinical implementation, it may be advantageous for a skin biopsy to be as small as possible as this minimizes disruption to a subject. The inventors have found that the measurements obtained in a 4 mm skin biopsy were comparable to a larger 6 mm biopsy when assessed 4 hours post-biopsy. Thus, in some embodiments, the size of the skin biopsy sample is about 3.5 mm to 4.5 mm. In some embodiments, the size of the skin biopsy sample is at least 4 mm. In some embodiments, the size of the skin biopsy sample is about 3.5 mm. In some embodiments, the size of the skin biopsy sample is about 3.6 mm. In some embodiments, the size of the skin biopsy sample is about 3.7 mm. In some embodiments, the size of the skin biopsy sample is about 3.8 mm. In some embodiments, the size of the skin biopsy sample is about 3.9 mm. In some embodiments, the size of the skin biopsy sample is about 4 mm. In some embodiments, the size of the skin biopsy sample is about 4.1 mm. In some embodiments, the size of the skin biopsy sample is about 4.2 mm. In some embodiments, the size of the skin biopsy sample is about 4.3 mm. In some embodiments, the size of the skin biopsy sample is about 4.4 mm. In some embodiments, the size of the skin biopsy sample is about 4.5 mm. In some embodiments, the size of the skin biopsy sample is about 4.6 mm. In some embodiments, the size of the skin biopsy sample is about 4.7 mm. In some embodiments, the size of the skin biopsy sample is about 4.8 mm. In some embodiments, the size of the skin biopsy sample is about 4.8 mm. In some embodiments, the size of the skin biopsy sample is about 4.9 mm. In some embodiments, the size of the skin biopsy sample is about 5.0 mm.

In one embodiment, the size of the skin sample is at least 4.0 mm. In another embodiment, the size of the skin sample is about 4.0 mm. In some embodiments, the biopsy sample (for example, a skin sample) obtained from the subject is cultured ex vivo. An inhibitor of IL1α can be administered to the sample (for example, a skin sample) prior to the ex vivo culturing. In some embodiments, an inhibitor of IL1α can be administered to the sample (for example, a skin sample) during the ex vivo culturing. In other embodiments, the sample (for example, a skin sample) is cultured ex vivo for a period of time prior to administering to the sample the inhibitor of IL1α.

In some embodiments, the levels of the one or more biomarkers provided herein can be measured at a time when IL1α would have been elevated should no inhibitor of IL1α be administrated (e.g., 4 to 24 hours post the biopsy procedure).

In one embodiment the levels of the one or more biomarkers provided herein are measured in a skin sample at 4 hours post the biopsy procedure. In certain such embodiments, the levels of one or more biomarkers provided herein are measured in a skin sample that has a size of at least 4.0 mm at 4 hours post the biopsy procedure. In other embodiments, the levels of one or more biomarkers provided herein are measured in a skin sample that has a size of about 4.0 mm at 4 hours post the biopsy procedure.

In another embodiment, the levels of the one or more biomarkers provided herein are measured in a skin sample at 24 hours post the biopsy procedure. In certain such embodiments, the levels of one or more biomarkers provided herein are measured in a skin sample that has a size of at least 4.0 mm at 24 hours post the biopsy procedure. In other embodiments, the levels of one or more biomarkers provided herein are measured in a skin sample that has a size of about 4.0 mm at 24 hours post the biopsy procedure.

In addition, a local anaesthetic that is used during a biopsy procedure may affect the ability of an IL1α inhibitor to reduce the level of one or more biomarkers. For example, as shown in Section 6 below, lidocaine injection affects ability of anti-IL1α to reduce level of GCSF, CXCL1 and IL8 in culture supernatant at 4-hour, but not at 24-hour time point. Therefore, the levels of the one or more biomarkers can be measured at least 4 hours post administration of the inhibitor of IL1α. In some embodiments, the levels of the one or more biomarkers are measured at least 5 hours post administration of the inhibitor of IL1α. In some embodiments, the levels of the one or more biomarkers are measured at least 6 hours post administration of the inhibitor of IL1α. In some embodiments, the levels of the one or more biomarkers are measured at least 7 hours post administration of the inhibitor of IL1α. In some embodiments, the levels of the one or more biomarkers are measured at least 8 hours post administration of the inhibitor of IL1α. In some embodiments, the levels of the one or more biomarkers are measured at least 9 hours post administration of the inhibitor of IL1α. In some embodiments, the levels of the one or more biomarkers are measured at least 10 hours post administration of the inhibitor of IL1α. In some embodiments, the levels of the one or more biomarkers are measured at least 11 hours post administration of the inhibitor of IL1α. In some embodiments, the levels of the one or more biomarkers are measured at least 12 hours post administration of the inhibitor of IL1α. In some embodiments, the levels of the one or more biomarkers are measured at least 13 hours post administration of the inhibitor of IL1α. In some embodiments, the levels of the one or more biomarkers are measured at least 14 hours post administration of the inhibitor of IL1α. In some embodiments, the levels of the one or more biomarkers are measured at least 15 hours post administration of the inhibitor of IL1α. In some embodiments, the levels of the one or more biomarkers are measured at least 16 hours post administration of the inhibitor of IL1α. In some embodiments, the levels of the one or more biomarkers are measured at least 17 hours post administration of the inhibitor of IL1α. In some embodiments, the levels of the one or more biomarkers are measured at least 18 hours post administration of the inhibitor of IL1α. In some embodiments, the levels of the one or more biomarkers are measured at least 19 hours post administration of the inhibitor of IL1α. In some embodiments, the levels of the one or more biomarkers are measured at least 20 hours post administration of the inhibitor of IL1α. In some embodiments, the levels of the one or more biomarkers are measured at least 21 hours post administration of the inhibitor of IL1α. In some embodiments, the levels of the one or more biomarkers are measured at least 22 hours post administration of the inhibitor of IL1α. In some embodiments, the levels of the one or more biomarkers are measured at least 23 hours post administration of the inhibitor of IL1α. In some embodiments, the levels of the one or more biomarkers are measured at least 24 hours post administration of the inhibitor of IL1α. In some embodiments, the levels of the one or more biomarkers are measured at least 25 hours post administration of the inhibitor of IL1α. In some embodiments, the levels of the one or more biomarkers are measured at least 30 hours post administration of the inhibitor of IL1α.

In one embodiment, the levels of the one or more biomarkers are measured at least 24 hours post administration of the inhibitor of IL1α(for example, an anti-IL1α antibody, such as bermekimab).

In certain embodiments, the levels of one or more biomarkers are measured at least 24 hours post administration of the inhibitor of IL1α(for example, an anti-IL1α antibody, such as bermekimab) and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post administration of the inhibitor of IL1α(for example, an anti-IL1α antibody, such as bermekimab). In another embodiment, the level of CXCL1 is measured at least 24 hours post administration of the inhibitor of IL1α(for example, an anti-IL1α antibody, such as bermekimab). In another embodiment, the level of IL6 is measured at least 24 hours post administration of the inhibitor of IL1α(for example, an anti-IL1α antibody, such as bermekimab).

In one embodiment, the levels of CSF3 (GCSF), CXCL1 and IL6 are measured at least 24 hours post administration of the inhibitor of IL1α(for example, an anti-IL1α antibody, such as bermekimab).

In one embodiment, the levels of GCSF, CXCL1, IL6 and IL8 are measured at least 24 hours post administration of the inhibitor of IL1α(for example, an anti-IL1α antibody, such as bermekimab).

In one embodiment, the levels of CXCL1, GCSF, GMCSF, IL6, IL8, MIP1a and TGFa are measured at least 24 hours post administration of the inhibitor of IL1α(for example, an anti-IL1α antibody, such as bermekimab).

In one embodiment, the levels of GCSF, CXCL1, IL4, IL6, IL8, MDC and IP10 are measured at least 24 hours post administration of the inhibitor of IL1α(for example, an anti-IL1α antibody, such as bermekimab).

In one embodiment, the levels of GCSF, CXCL1, IL4, IL6, IL8, MDC, IP10, GMCSF, MIP1a and TGFa are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, and PTGS2 are measured at least 24 hours post administration of the inhibitor of IL1α(for example, an anti-IL1α antibody, such as bermekimab).

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post administration of the inhibitor of IL1α(for example, an anti-IL1α antibody, such as bermekimab).

In one embodiment, the levels of CCL20, CSF3(GCSF), CXCL1, CXCL2, CXCL3, CXCL5, IL6, IL8, and PTGS2 are measured at least 24 hours post administration of the inhibitor of IL1α(for example, an anti-IL1α antibody, such as bermekimab).

The time point at which the biomarkers are measured may also be defined according to the time elapsed since the biopsy procedure was performed. As discussed above, a local anaesthetic that is used in the biopsy procedure may affect the ability of an IL1α inhibitor to reduce the level of one or more biomarkers. Thus, in some embodiments, the levels of the one or more biomarkers are measured at least 4 hours post the biopsy procedure. In some embodiments, the levels of the one or more biomarkers are measured at least 5 hours post the biopsy procedure. In some embodiments, the levels of the one or more biomarkers are measured at least 6 hours post the biopsy procedure. In some embodiments, the levels of the one or more biomarkers are measured at least 7 hours post the biopsy procedure. In some embodiments, the levels of the one or more biomarkers are measured at least 8 hours post the biopsy procedure. In some embodiments, the levels of the one or more biomarkers are measured at least 9 hours post the biopsy procedure. In some embodiments, the levels of the one or more biomarkers are measured at least 10 hours post the biopsy procedure. In some embodiments, the levels of the one or more biomarkers are measured at least 11 hours post the biopsy procedure. In some embodiments, the levels of the one or more biomarkers are measured at least 12 hours post the biopsy procedure. In some embodiments, the levels of the one or more biomarkers are measured at least 13 hours post the biopsy procedure. In some embodiments, the levels of the one or more biomarkers are measured at least 14 hours post the biopsy procedure. In some embodiments, the levels of the one or more biomarkers are measured at least 15 hours post the biopsy procedure. In some embodiments, the levels of the one or more biomarkers are measured at least 16 hours post the biopsy procedure. In some embodiments, the levels of the one or more biomarkers are measured at least 17 hours post the biopsy procedure. In some embodiments, the levels of the one or more biomarkers are measured at least 18 hours post the biopsy procedure. In some embodiments, the levels of the one or more biomarkers are measured at least 19 hours post the biopsy procedure. In some embodiments, the levels of the one or more biomarkers are measured at least 20 hours post the biopsy procedure. In some embodiments, the levels of the one or more biomarkers are measured at least 21 hours post the biopsy procedure. In some embodiments, the levels of the one or more biomarkers are measured at least 22 hours post the biopsy procedure. In some embodiments, the levels of the one or more biomarkers are measured at least 23 hours post the biopsy procedure. In some embodiments, the levels of the one or more biomarkers are measured at least 24 hours post the biopsy procedure. In some embodiments, the levels of the one or more biomarkers are measured at least 25 hours post the biopsy procedure. In some embodiments, the levels of the one or more biomarkers are measured at least 30 hours post the biopsy procedure.

In one embodiment, the levels of the one or more biomarkers are measured at least 24 hours post the biopsy procedure.

In certain embodiments, the levels of one or more biomarkers are measured at least 24 hours post the biopsy procedure and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post the biopsy procedure. In another embodiment, the level of CXCL1 is measured at least 24 hours post the biopsy procedure. In another embodiment, the level of IL6 is measured at least 24 hours post the biopsy procedure.

In one embodiment, the levels of CSF3 (GCSF), CXCL1 and IL6 are measured at least 24 hours post the biopsy procedure.

In one embodiment, the levels of GCSF, CXCL1, IL6 and IL8 are measured at least 24 hours post the biopsy procedure.

In one embodiment, the levels of CXCL1, GCSF, GMCSF, IL6, IL8, MIP1a and TGFa are measured at least 24 hours post the biopsy procedure.

In one embodiment, the levels of GCSF, CXCL1, IL4, IL6, IL8, MDC and IP10 are measured at least 24 hours post the biopsy procedure.

In one embodiment, the levels of GCSF, CXCL1, IL4, IL6, IL8, MDC, IP10, GMCSF, MIP1a and TGFa are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, and PTGS2 are measured at least 24 hours post the biopsy procedure.

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post the biopsy procedure.

In one embodiment, the levels of CCL20, CSF3(GCSF), CXCL1, CXCL2, CXCL3, CXCL5, IL6, IL8, and PTGS2 are measured at least 24 hours post the biopsy procedure).

The effects of the inhibitor of IL1α(for example, an anti-IL1α antibody, such as bermekimab) can be determined by comparing the levels of the one or more biomarkers provided herein with reference levels of these biomarkers.

The reference levels of these biomarkers can be the levels of these biomarkers in a reference injured skin sample (for example, a skin biopsy sample) without any influence by an inhibitor of IL1α(for example, an anti-IL1α antibody, such as bermekimab), and the levels of the biomarkers in the sample and the levels of the biomarkers in the reference sample are measured at the same time point (for example, at least 4 hours or at least 24 hours post the biopsy procedure).

In some embodiments, the reference levels of the one or more biomarkers are the levels of the one or more biomarkers in a reference sample from the subject prior to administration of the inhibitor of IL1α. In some embodiments, the reference levels of the one or more biomarkers are the levels of the one or more biomarkers in a reference sample from the subject without administration of the inhibitor of IL1α. In some embodiments, the reference levels of the one or more biomarkers are pre-determined levels of the one or more biomarkers. In some embodiments, the reference levels of the one or more biomarkers are the levels of the one or more biomarkers in a reference sample administered with a control agent (e.g., a positive control agent that inhibits IL1α, such as the anti-IL1α antibody R&D, clone 4414, or a negative control agent that does not inhibit IL1α).

In one embodiment, the reference levels of the one or more biomarkers are the levels of the one or more biomarkers in a reference skin biopsy sample from the subject without administration of the inhibitor of IL1α.

When the levels of the biomarkers are compared to the references levels that represent the elevated levels of these biomarkers post injury (e.g. biopsy induced injury) but without the influence of an inhibitor of IL1α, the lower levels of the one or more biomarkers as compared with the reference levels indicate that the inhibitor is effective in inhibiting IL1α.

In some embodiments, the inhibitor is identified as effective in inhibiting IL1α if the levels of the one or more biomarkers in the sample are at least 20%, are at least 30%, at least 35%, at least 40%, or at least 50% less than the reference levels.

In one embodiment, the inhibitor is identified as effective in inhibiting IL1α if the levels of the one or more cytokine biomarkers secreted by the sample (for example, as measured by a Luminex assay) are at least 50% less than the reference levels. In another embodiment, the inhibitor is identified as effective in inhibiting IL1α if the mRNA levels (for example, as measured by a Nanostring assay) of the one or more biomarkers in the sample are at least 30% less than the reference levels. In another embodiment, the inhibitor is identified as effective in inhibiting IL1α if the mRNA levels (for example, as measured by an RNAseq assay) of the one or more biomarkers in the sample are at least 20% less than the reference levels.

In another aspect, a pharmacodynamics or pharmacokinetic effect of an inhibitor of IL1α is determined by administering the inhibitor directly to the subject. The subjects who received an effective IL1α inhibitor may be protected from injury-mediated upregulation (e.g. by biopsy procedure) of IL1α and subsequent downstream effects.

Thus, in some more embodiments, provided herein is a method for determining a pharmacodynamics or pharmacokinetic effect of an inhibitor of IL1α, comprises: i. obtaining a first sample from a subject; ii. administering an IL1α inhibitor to the subject; iii. obtaining a second sample from the subject; iv. measuring levels of one or more biomarkers in the first sample and the second sample; and v. determining that the treatment is effective when the levels of the one or more biomarkers in the second sample are lower than the levels of one or more biomarkers in the first sample. In some embodiments, the first sample and the second sample are from the same source.

In some embodiments, the second sample is obtained from the subject at 1.5 to 2.5 hours post the administration of the IL1α inhibitor to the subject. In some embodiments, the second sample is obtained from the subject at about 1.5 hours post the administration. In some embodiments, the second sample is obtained from the subject at about 1.6 hours post the administration. In some embodiments, the second sample is obtained from the subject at about 1.7 hours post the administration. In some embodiments, the second sample is obtained from the subject at about 1.8 hours post the administration. In some embodiments, the second sample is obtained from the subject at about 1.9 hours post the administration. In some embodiments, the second sample is obtained from the subject at about 2.0 hours post the administration. In some embodiments, the second sample is obtained from the subject at about 2.1 hours post the administration. In some embodiments, the second sample is obtained from the subject at about 2.2 hours post the administration. In some embodiments, the second sample is obtained from the subject at about 2.3 hours post the administration. In some embodiments, the second sample is obtained from the subject at about 2.4 hours post the administration. In some embodiments, the second sample is obtained from the subject at about 2.5 hours post the administration.

The first and second samples may contain skin cells. In some embodiments, the sample is obtained by biopsy procedure and contain injured skin cells. In some embodiments, the size of the skin biopsy sample is about 3.5 mm to 4.5 mm. In some embodiments, the size of the skin biopsy sample is at least 4 mm. In some embodiments, the size of the skin biopsy sample is about 3.5 mm. In some embodiments, the size of the skin biopsy sample is about 3.6 mm. In some embodiments, the size of the skin biopsy sample is about 3.7 mm. In some embodiments, the size of the skin biopsy sample is about 3.8 mm. In some embodiments, the size of the skin biopsy sample is about 3.9 mm. In some embodiments, the size of the skin biopsy sample is about 4 mm. In some embodiments, the size of the skin biopsy sample is about 4.1 mm. In some embodiments, the size of the skin biopsy sample is about 4.2 mm. In some embodiments, the size of the skin biopsy sample is about 4.3 mm. In some embodiments, the size of the skin biopsy sample is about 4.4 mm. In some embodiments, the size of the skin biopsy sample is about 4.5 mm. In some embodiments, the size of the skin biopsy sample is about 4.6 mm. In some embodiments, the size of the skin biopsy sample is about 4.7 mm. In some embodiments, the size of the skin biopsy sample is about 4.8 mm. In some embodiments, the size of the skin biopsy sample is about 4.8 mm. In some embodiments, the size of the skin biopsy sample is about 4.9 mm. In some embodiments, the size of the skin biopsy sample is about 5.0 mm.

In one embodiment, the size of the skin sample is at least 4.0 mm. In another embodiment, the size of the skin sample is about 4.0 mm.

In some embodiments, the biopsy sample obtained from the subject is cultured ex vivo.

In other embodiments, the sample used in the methods provided herein may comprise body fluids from a subject. In some embodiments, the sample is a blood sample. More detailed description of a sample is provided in Section 5.3 below.

In some embodiments, the levels of the one or more biomarkers provided herein can be measured at a time when IL1α would have been elevated should no inhibitor of IL1α be administrated (e.g., 4 to 24 hours post the biopsy procedure). In some embodiments, the levels of the one or more biomarkers are measured at least 4 hours post the biopsy procedure. In some embodiments, the levels of the one or more biomarkers are measured at least 5 hours post the biopsy procedure. In some embodiments, the levels of the one or more biomarkers are measured at least 6 hours post the biopsy procedure. In some embodiments, the levels of the one or more biomarkers are measured at least 7 hours post the biopsy procedure. In some embodiments, the levels of the one or more biomarkers are measured at least 8 hours post the biopsy procedure. In some embodiments, the levels of the one or more biomarkers are measured at least 9 hours post the biopsy procedure. In some embodiments, the levels of the one or more biomarkers are measured at least 10 hours post the biopsy procedure. In some embodiments, the levels of the one or more biomarkers are measured at least 11 hours post the biopsy procedure. In some embodiments, the levels of the one or more biomarkers are measured at least 12 hours post the biopsy procedure. In some embodiments, the levels of the one or more biomarkers are measured at least 13 hours post the biopsy procedure. In some embodiments, the levels of the one or more biomarkers are measured at least 14 hours post the biopsy procedure. In some embodiments, the levels of the one or more biomarkers are measured at least 15 hours post the biopsy procedure. In some embodiments, the levels of the one or more biomarkers are measured at least 16 hours post the biopsy procedure. In some embodiments, the levels of the one or more biomarkers are measured at least 17 hours post the biopsy procedure. In some embodiments, the levels of the one or more biomarkers are measured at least 18 hours post the biopsy procedure. In some embodiments, the levels of the one or more biomarkers are measured at least 19 hours post the biopsy procedure. In some embodiments, the levels of the one or more biomarkers are measured at least 20 hours post the biopsy procedure. In some embodiments, the levels of the one or more biomarkers are measured at least 21 hours post the biopsy procedure. In some embodiments, the levels of the one or more biomarkers are measured at least 22 hours post the biopsy procedure. In some embodiments, the levels of the one or more biomarkers are measured at least 23 hours post the biopsy procedure. In some embodiments, the levels of the one or more biomarkers are measured at least 24 hours post the biopsy procedure. In some embodiments, the levels of the one or more biomarkers are measured at least 25 hours post the biopsy procedure. In some embodiments, the levels of the one or more biomarkers are measured at least 30 hours post the biopsy procedure.

In one embodiment, the levels of the one or more biomarkers are measured at least 4 hours post the biopsy procedure. In another embodiment, the levels of the one or more biomarkers are measured at least 24 hours post the biopsy procedure.

In some embodiments, the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one ore more biomarkers comprise CXCL1. In another embodiment, the one or more biomarkers comprise IL6.

In some embodiments, the method provided herein comprises measuring all of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. This approach may allow variations of individual gene expression in different subjects. Similarly, this method also applies to a subset including 2 or more biomarkers selected from CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In one embodiment, the one or more biomarkers are CSF3 (GCSF), CXCL1 and IL6. In one embodiment, a composite score is calculated based on the levels of CSF3 (GCSF), CXCL1 and IL6, and wherein the method further comprises comparing the composite score to a reference score.

The levels of the biomarkers provided herein can be measured by determining protein levels or nucleic acid levels (e.g., mRNA, DNA or cDNA levels) of these biomarkers. In one embodiment, the levels of the biomarkes provided herein are measured by determining protein levels of these biomarkers. In another embodiment, the levels of the biomarkers provided herein are measured by determining mRNA levels of these biomarkers. Detailed description of these measurement methods are provided in the following sections.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In some embodiments, the method provided herein comprises measuring all of GCSF, CXCL1, IL4, IL6, IL8, MDC, IP10, GMCSF, MIP1a and TGFa. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. Similarly, this method also applies to a subset including 2 or more biomarkers selected from GCSF, CXCL1, IL4, IL6, IL8, MDC, IP10, GMCSF, MIP1a and TGFa.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In one embodiment, the one or more biomarkers are CSF3 (GCSF), CXCL1 and IL6. In one embodiment, a composite score is calculated based on the levels of CSF3 (GCSF), CXCL1 and IL6, and wherein the method further comprises comparing the composite score to a reference score.

In some embodiments, the levels of the biomarkers provided herein can be measured by determining protein levels of these biomarkers. Detailed description of these measurement methods are provided in the following sections.

In some embodiments of the various methods provided herein, the level(s) of the one or more biomarker(s) post treatment with the IL1α inhibitor is compared with the reference level(s) of the same biomarker(s) from a sample cultured for the same amount of time without the treatment, and compared with another reference level(s) of the same biomarker(s) in a control sample. In some embodiments, the control sample is a positive control using another IL1α inhibitor (for example, the anti-IL1α antibody R&D, clone 4414). In other embodiments, the control sample is a negative control using a compound that is not an IL1α inhibitor. In yet other embodiments, another reference level(s) of the same biomarker(s) in a control sample is the level(s) of the biomarker(s) in a control sample just obtained from the subject, e.g., within 30 mins after obtaining the sample from the subject or within the period before IL1α level is elevated.

In some embodiments, the various methods provided herein can be used to measure PD and/or PK effects of an anti-IL1α treatment in healthy subjects. In other embodiments, the various methods provided herein can be used to measure PD and/or PK effects of an anti-IL1α treatment in a subject having atopic dermatitis, hidradenitis suppurativa, psoriasis, cutaneous lupus erythematosus, or autoimmune bullous disease. In one embodiment, the various methods provided herein are used to measure PD and/or PK effects of an anti-IL1α treatment in a subject having atopic dermatitis. In another embodiment, the various methods provided herein are used to measure PD and/or PK effects of an anti-IL1α treatment in a subject having hidradenitis suppurativa.

### 5.2.2. Biomarkers for Predicting Responses to Treatments and Selective Treatments Using Biomarkers

The methods and biomarkers provided herein can be used to predict and evaluate the responsiveness to a treatment comprising an inhibitor of IL1α(for example an anti-IL1α antibody, such as bermekimab). In some embodiments, provided herein is a method of identifying a subject who is likely to be responsive to a treatment comprising an inhibitor of IL1α(for example an anti-IL1α antibody, such as bermekimab). In other embodiments, provided herein is a method of predicting the responsiveness of a subject to a treatment comprising an inhibitor of IL1α(for example an anti-IL1α antibody, such as bermekimab). In other embodiments, provided herein is a method of monitoring the response of a subject to a treatment comprising an inhibitor of IL1α(for example an anti-IL1α antibody, such as bermekimab). In yet other embodiments, provided herein is a method for determining dosing regimens of an inhibitor of IL1α(for example an anti-IL1α antibody, such as bermekimab).

In some embodiments, the method provided herein comprises contacting an inhibitor of IL1α(for example an anti-IL1α antibody, such as bermekimab) with a sample ex vivo. In other embodiments, the method provided herein comprises administering an inhibitor of IL1α(for example an anti-IL1α antibody, such as bermekimab) directly to a subject for predicting or monitoring responsiveness of the subject to the inhibitor of IL1α(for example an anti-IL1α antibody, such as bermekimab).

In one aspect, provided herein is a method for monitoring the response of a subject to a treatment comprising an inhibitor of IL1α(for example an anti-IL1α antibody, such as bermekimab) comprising providing a sample from the subject; administering the inhibitor of IL1α(for example an anti-IL1α antibody, such as bermekimab) to the sample; measuring levels of one or more biomarkers in the sample; and identifying the subject as being likely to be responsive to the treatment comprising the inhibitor of IL1α(for example an anti-IL1α antibody, such as bermekimab), predicting the responsiveness of the subject to the treatment comprising the inhibitor of IL1α(for example an anti-IL1α antibody, such as bermekimab), or monitoring the response of the subject to a treatment comprising the inhibitor of IL1α (for example an anti-IL1α antibody, such as bermekimab), based on the levels of the one or more biomarkers.

The effects of the inhibitor of IL1α(for example an anti-IL1α antibody, such as bermekimab) can be determined by comparing the levels of the one or more biomarkers provided herein with reference levels of these biomarkers.

The reference levels of these biomarkers can be the levels of these biomarkers in a reference injured skin sample (for example a skin biopsy sample) without any influence by an inhibitor of IL1α(for example an anti-IL1α antibody, such as bermekimab), and the levels of the biomarkers in the sample and the levels of the biomarkers in the reference sample are measured at the same time point (for example, at least 4 hours or at least 24 hours post the biopsy procedure).

In some embodiments, the reference levels of the one or more biomarkers are the levels of the one or more biomarkers in a reference sample from the subject prior to administration of the inhibitor of IL1α(for example an anti-IL1α antibody, such as bermekimab). In some embodiments, the reference levels of the one or more biomarkers are the levels of the one or more biomarkers in a reference sample from the subject without administration of the inhibitor of IL1α(for example an anti-IL1α antibody, such as bermekimab). In some embodiments, the reference levels of the one or more biomarkers are pre-determined levels of the one or more biomarkers. In some embodiments, the reference levels of the one or more biomarkers are the levels of the one or more biomarkers in a reference sample administered with a control agent (e.g., a positive control agent that inhibits IL1α, such as the anti-IL1α antibody R&D, clone 4414, or a negative control agent that does not inhibit IL1α).

In one embodiment, the reference levels of the one or more biomarkers are the levels of the one or more biomarkers in a reference skin biopsy sample from the subject without administration of the inhibitor of IL1α, and the levels of the biomarkers in the sample and the levels of the biomarkers in the reference sample are measured at the same time point.

When the levels of the biomarkers are compared to the references level that represent the elevated levels of these biomarkers post injury (e.g. biopsy induced injury) but without the influence of an inhibitor of IL1α(for example an anti-IL1α antibody, such as bermekimab), the lower levels of the one or more biomarkers as compared with the references indicate that subject is likely to be responsive to the treatment comprising the inhibitor of IL1α (for example an anti-IL1α antibody, such as bermekimab).

In some embodiments, the subject is identified likely to be responsive to the treatment comprising the inhibitor of IL1α if the levels of the one or more biomarkers in the sample are at least 20%, at least 30%, at least 35%, at least 40%, or at least 50% less than the reference levels.

In one embodiment, the subject is identified likely to be responsive to the treatment comprising the inhibitor of IL1α if the levels of the one or more cytokine biomarkers secreted by the sample (for example, as measured by a Luminex assay) are at least 50% less than the reference levels. In another embodiment, the subject is identified likely to be responsive to the treatment comprising the inhibitor of IL1α if the mRNA levels (for example, as measured by a Nanostring assay) of the one or more biomarkers in the sample are at least 30% less than the reference levels. In another embodiment, the subject is identified likely to be responsive to the treatment comprising the inhibitor of IL1α if the mRNA levels (for example, as measured by an RNAseq assay) of the one or more biomarkers in the sample are at least 20% less than the reference levels.

In some embodiments, the sample is obtained by biopsy procedure and contain injured skin cells. In some embodiments, the size of the skin biopsy sample is about 3.5 mm to 4.5 mm. In some embodiments, the size of the skin biopsy sample is at least 4 mm. In some embodiments, the size of the skin biopsy sample is about 3.5 mm. In some embodiments, the size of the skin biopsy sample is about 3.6 mm. In some embodiments, the size of the skin biopsy sample is about 3.7 mm. In some embodiments, the size of the skin biopsy sample is about 3.8 mm. In some embodiments, the size of the skin biopsy sample is about 3.9 mm. In some embodiments, the size of the skin biopsy sample is about 4 mm. In some embodiments, the size of the skin biopsy sample is about 4.1 mm. In some embodiments, the size of the skin biopsy sample is about 4.2 mm. In some embodiments, the size of the skin biopsy sample is about 4.3 mm. In some embodiments, the size of the skin biopsy sample is about 4.4 mm. In some embodiments, the size of the skin biopsy sample is about 4.5 mm. In some embodiments, the size of the skin biopsy sample is about 4.6 mm. In some embodiments, the size of the skin biopsy sample is about 4.7 mm. In some embodiments, the size of the skin biopsy sample is about 4.8 mm. In some embodiments, the size of the skin biopsy sample is about 4.8 mm. In some embodiments, the size of the skin biopsy sample is about 4.9 mm. In some embodiments, the size of the skin biopsy sample is about 5.0 mm.

In one embodiment, the size of the skin sample is at least 4.0 mm. In another embodiment, the size of the skin sample is about 4.0 mm.

In other embodiments, the sample used in the methods provided herein may comprises body fluids from a subject. In some embodiments, the sample is a blood sample. More detailed description of a sample is provided in Section 5.3 below.

In some embodiments, the biopsy sample (for example, a skin sample) obtained from the subject is cultured ex vivo. An inhibitor of IL1α can be administered to the sample (for example, a skin sample) prior to the ex vivo culturing. In some embodiments, an inhibitor of IL1α can be administered to the sample (for example, a skin sample) during the ex vivo culturing. In other embodiments, the sample (for example, a skin sample) is cultured ex vivo for a period of time prior to administering to the sample the inhibitor of IL1α.

In some embodiments, the levels of the one or more biomarkers provided herein can be measured at a time when IL1α would have been elevated should no inhibitor of IL1α be administrated (e.g., 4 to 24 hours post the biopsy procedure).

In some embodiments the levels of the one or more biomarkers provided herein are measured in a skin sample at 4 hours post the biopsy procedure. In certain such embodiments, the levels of one or more biomarkers provided herein are measured in a skin sample that has a size of at least 4.0 mm at 4 hours post the biopsy procedure. In other embodiments, the levels of one or more biomarkers provided herein are measured in a skin sample that has a size of about 4.0 mm at 4 hours post the biopsy procedure.

In another emboimdent, the levels of the one or more biomarkers provided herein are measuered in a skin sample at 24 hours post the biopsy procedure. In certain such embodiments, the levels of one or more biomarkers provided herein are measured in a skin sample that has a size of at least 4.0 mm at 24 hours post the biopsy procedure. In other embodiments, the levels of one or more biomarkers provided herein are measured in a skin sample that has a size of about 4.0 mm at 24 hours post the biopsy procedure.

The levels of the one or more biomarkers can be measured at least 4 hours post administration of the inhibitor of IL1α. In some embodiments, the levels of the one or more biomarkers are measured at least 5 hours post administration of the inhibitor of IL1α. In some embodiments, the levels of the one or more biomarkers are measured at least 6 hours post administration of the inhibitor of IL1α. In some embodiments, the levels of the one or more biomarkers are measured at least 7 hours post administration of the inhibitor of IL1α. In some embodiments, the levels of the one or more biomarkers are measured at least 8 hours post administration of the inhibitor of IL1α. In some embodiments, the levels of the one or more biomarkers are measured at least 9 hours post administration of the inhibitor of IL1α. In some embodiments, the levels of the one or more biomarkers are measured at least 10 hours post administration of the inhibitor of IL1α. In some embodiments, the levels of the one or more biomarkers are measured at least 11 hours post administration of the inhibitor of IL1α. In some embodiments, the levels of the one or more biomarkers are measured at least 12 hours post administration of the inhibitor of IL1α. In some embodiments, the levels of the one or more biomarkers are measured at least 13 hours post administration of the inhibitor of IL1α. In some embodiments, the levels of the one or more biomarkers are measured at least 14 hours post administration of the inhibitor of IL1α. In some embodiments, the levels of the one or more biomarkers are measured at least 15 hours post administration of the inhibitor of IL1α. In some embodiments, the levels of the one or more biomarkers are measured at least 16 hours post administration of the inhibitor of IL1α. In some embodiments, the levels of the one or more biomarkers are measured at least 17 hours post administration of the inhibitor of IL1α. In some embodiments, the levels of the one or more biomarkers are measured at least 18 hours post administration of the inhibitor of IL1α. In some embodiments, the levels of the one or more biomarkers are measured at least 19 hours post administration of the inhibitor of IL1α. In some embodiments, the levels of the one or more biomarkers are measured at least 20 hours post administration of the inhibitor of IL1α. In some embodiments, the levels of the one or more biomarkers are measured at least 21 hours post administration of the inhibitor of IL1α. In some embodiments, the levels of the one or more biomarkers are measured at least 22 hours post administration of the inhibitor of IL1α. In some embodiments, the levels of the one or more biomarkers are measured at least 23 hours post administration of the inhibitor of IL1α. In some embodiments, the levels of the one or more biomarkers are measured at least 24 hours post administration of the inhibitor of IL1α. In some embodiments, the levels of the one or more biomarkers are measured at least 25 hours post administration of the inhibitor of IL1α. In some embodiments, the levels of the one or more biomarkers are measured at least 30 hours post administration of the inhibitor of IL1α.

In one embodiment, the levels of the one or more biomarkers are measured at least 24 hours post administration of the inhibitor of IL1α(for example, an anti-IL1α antibody, such as bermekimab).

In certain embodiments, the levels of one or more biomarkers are measured at least 24 hours post administration of the inhibitor of IL1α and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAIVID1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post administration of the inhibitor of IL1α(for example, an anti-IL1α antibody, such as bermekimab). In another embodiment, the level of CXCL1 is measured at least 24 hours post administration of the inhibitor of IL1α(for example, an anti-IL1α antibody, such as bermekimab). In another embodiment, the level of IL6 is measured at least 24 hours post administration of the inhibitor of IL1α(for example, an anti-IL1α antibody, such as bermekimab).

In one embodiment, the levels of CSF3 (GCSF), CXCL1 and IL6 are measured at least 24 hours post administration of the inhibitor of IL1α(for example, an anti-IL1α antibody, such as bermekimab).

In one embodiment, the levels of GCSF, CXCL1, IL6 and IL8 are measured at least 24 hours post administration of the inhibitor of IL1α(for example, an anti-IL1α antibody, such as bermekimab).

In one embodiment, the levels of CXCL1, GCSF, GMCSF, IL6, IL8, MIP1a and TGFa are measured at least 24 hours post administration of the inhibitor of IL1α(for example, an anti-IL1α antibody, such as bermekimab).

In one embodiment, the levels of GCSF, CXCL1, IL4, IL6, IL8, MDC and IP10 are measured at least 24 hours post administration of the inhibitor of IL1α(for example, an anti-IL1α antibody, such as bermekimab).

In one embodiment, the levels of GCSF, CXCL1, IL4, IL6, IL8, MDC, IP10, GMCSF, MIP1a and TGFa are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, and PTGS2 are measured at least 24 hours post administration of the inhibitor of IL1α(for example, an anti-IL1α antibody, such as bermekimab).

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post administration of the inhibitor of IL1α(for example, an anti-IL1α antibody, such as bermekimab).

In one embodiment, the levels of CCL20, CSF3(GCSF), CXCL1, CXCL2, CXCL3, CXCL5, IL6, IL8, and PTGS2 are measured at least 24 hours post administration of the inhibitor of IL1α(for example, an anti-IL1α antibody, such as bermekimab).

In some embodiments, the levels of the one or more biomarkers are measured at least 4 hours post the biopsy procedure. In some embodiments, the levels of the one or more biomarkers are measured at least 5 hours post the biopsy procedure. In some embodiments, the levels of the one or more biomarkers are measured at least 6 hours post the biopsy procedure. In some embodiments, the levels of the one or more biomarkers are measured at least 7 hours post the biopsy procedure. In some embodiments, the levels of the one or more biomarkers are measured at least 8 hours post the biopsy procedure. In some embodiments, the levels of the one or more biomarkers are measured at least 9 hours post the biopsy procedure. In some embodiments, the levels of the one or more biomarkers are measured at least 10 hours post the biopsy procedure. In some embodiments, the levels of the one or more biomarkers are measured at least 11 hours post the biopsy procedure. In some embodiments, the levels of the one or more biomarkers are measured at least 12 hours post the biopsy procedure. In some embodiments, the levels of the one or more biomarkers are measured at least 13 hours post the biopsy procedure. In some embodiments, the levels of the one or more biomarkers are measured at least 14 hours post the biopsy procedure. In some embodiments, the levels of the one or more biomarkers are measured at least 15 hours post the biopsy procedure. In some embodiments, the levels of the one or more biomarkers are measured at least 16 hours post the biopsy procedure. In some embodiments, the levels of the one or more biomarkers are measured at least 17 hours post the biopsy procedure. In some embodiments, the levels of the one or more biomarkers are measured at least 18 hours post the biopsy procedure. In some embodiments, the levels of the one or more biomarkers are measured at least 19 hours post the biopsy procedure. In some embodiments, the levels of the one or more biomarkers are measured at least 20 hours post the biopsy procedure. In some embodiments, the levels of the one or more biomarkers are measured at least 21 hours post the biopsy procedure. In some embodiments, the levels of the one or more biomarkers are measured at least 22 hours post the biopsy procedure. In some embodiments, the levels of the one or more biomarkers are measured at least 23 hours post the biopsy procedure. In some embodiments, the levels of the one or more biomarkers are measured at least 24 hours post the biopsy procedure. In some embodiments, the levels of the one or more biomarkers are measured at least 25 hours post the biopsy procedure. In some embodiments, the levels of the one or more biomarkers are measured at least 30 hours post the biopsy procedure.

In one embodiment, the levels of the one or more biomarkers are measured at least 24 hours post the biopsy procedure.

In certain embodiments, the levels of one or more biomarkers are measured at least 24 hours post the biopsy procedure and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post the biopsy procedure. In another embodiment, the level of CXCL1 is measured at least 24 hours post the biopsy procedure. In another embodiment, the level of IL6 is measured at least 24 hours post the biopsy procedure.

In one embodiment, the levels of CSF3 (GCSF), CXCL1 and IL6 are measured at least 24 hours post the biopsy procedure.

In one embodiment, the levels of GCSF, CXCL1, IL6 and IL8 are measured at least 24 hours post the biopsy procedure.

In one embodiment, the levels of CXCL1, GCSF, GMCSF, IL6, IL8, MIP1a and TGFa are measured at least 24 hours post the biopsy procedure.

In one embodiment, the levels of GCSF, CXCL1, IL4, IL6, IL8, MDC and IP10 are measured at least 24 hours post the biopsy procedure.

In one embodiment, the levels of GCSF, CXCL1, IL4, IL6, IL8, MDC, IP10, GMCSF, MIP1a and TGFa are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, and PTGS2 are measured at least 24 hours post the biopsy procedure.

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post the biopsy procedure.

In one embodiment, the levels of CCL20, CSF3(GCSF), CXCL1, CXCL2, CXCL3, CXCL5, IL6, IL8, and PTGS2 are measured at least 24 hours post the biopsy procedure).

In another aspect, the biomarkers provided herein and changes thereof upon treatment with an IL1α inhibitor (e.g., decreased levels of the biomarkers upon treatment) can be used for monitoring the response of a subject having an IL1α mediated disease to an IL1α inhibitor or determining an effective dose of an IL1α inhibitor to be administered to a subject having an IL1α mediated disease.

Therefore, in some embodiments, provided herein is a method for monitoring the response of a subject having an IL1α mediated disease to an IL1α inhibitor or determining an effective dose of an IL1α inhibitor to be administered to a subject having an IL1α mediated disease comprising administering an IL1α inhibitor to the subject; obtain a sample from the subject; measuring levels of one or more biomarkers in the sample; and monitoring the response and/or determining the effectiveness of the dose based on the levels of the one or more biomarkers. In some embodiments, the levels of the one or more biomarkers are compared with reference levels of the one or more biomarkers, thereby monitoring the response and/or determining the effectiveness of the dose. In some embodiments, the reference levels are pre-determined levels. In other embodiments, the reference levels are the levels of the one or more biomarkers prior to the treatment.

In one embodiment, the reference levels of the one or more biomarkers are the levels of the one or more biomarkers in a reference skin biopsy sample from the subject prior to administration of the inhibitor of IL1α.

The subjects who received an effective IL1α inhibitor may be protected from injury-mediated upregulation (e.g. by biopsy procedure) of IL1α and subsequent downstream effects. Thus, in some more specific embodiments, the method provided herein comprises: i. obtaining a first sample from a subject; ii. administering an IL1α inhibitor to the subject; iii. obtaining a second sample from the subject; iv. measuring levels of one or more biomarkers in the first sample and the second sample; and v. determining that the treatment is effective when the levels of the one or more biomarkers in the second sample are lower than the levels of one or more biomarkers in the first sample. In some embodiments, the first sample and the second sample are from the same source.

In some embodiments, the IL1α inhibitor is an anti-IL1α antibody, such as bermekimab.

In some embodiments, the IL1α mediated disease is atopic dermatitis, hidradenitis suppurativa, psoriasis, cutaneous lupus erythematosus, or autoimmune bullous disease. In one embodiment, the IL1α mediated disease is atopic dermatitis. In another embodiment, the IL1α mediated disease is hidradenitis suppurativa.

Thus, in some embodiments, the biomarkers provided herein and changes thereof upon treatment with an anti-IL1α antibody (such as bermekimab) can be used for monitoring the response of a subject having atopic dermatitis to an anti-IL1α antibody (such as bermekimab) or determining an effective dose of an anti-IL1α antibody (such as bermekimab) to be administered to a subject having atopic dermatitis.

In other embodiments, the biomarkers provided herein and changes thereof upon treatment with an anti-IL1α antibody (such as bermekimab) can be used for monitoring the response of a subject having hidradenitis suppurativa to an anti-IL1α antibody (such as bermekimab) or determining an effective dose of an anti-IL1α antibody (such as bermekimab) to be administered to a subject having hidradenitis suppurativa. In certain embodiments, the one or more biomarkers are selected from the group consisting of GCSF (CSF3), CXCL1, IL6, IL8, IL4, MDC, IP10, GMCSF (CSF2), MMP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the one or more biomarkers comprise CSF3 (GCSF). In one embodiment, the one or more biomarkers comprise CXCL1. In one embodiment, the one or more biomarkers comprise IL6.

In one embodiment, the one or more biomarkers comprise CSF3 (GCSF), CXCL1 and IL6.

In one embodiment, the one or more biomarkers comprise GCSF, CXCL1, IL6 and IL8.

In one embodiment, the one or more biomarkers comprise CXCL1, GCSF, GMCSF, IL6, IL8, MIP1a and TGFa.

In one embodiment, the one or more biomarkers comprise GCSF, CXCL1, IL4, IL6, IL8, MDC and IP10.

In one embodiment, the one or more biomarkers comprise GCSF, CXCL1, IL4, IL6, IL8, MDC, IP10, GMCSF, MIP1a and TGFa.

In one embodiment, the one or more biomarkers comprise CCL20, CSF3(GCSF), CXCL1, CXCL2, CXCL3, CXCL5, IL6, IL8, and PTGS2.

In one embodiment, the one or more biomarkers comprise CCL20, CCL3, CCL4, CCL8, CFB, CSF3(GCSF), CXCL1, CXCL2, CXCL3, CXCL5, IL1B, IL6, IL8, MMP3, and PTGS2.

In one embodiment, the one or more biomarkers comprise CCL20, CCL22, CSF3(GCSF), CXCL1, CXCL2, CXCL3, CXCL5, CXCL6, IL6, IL8, and PTGS2.

In one embodiment, the one or more biomarkers comprise CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the second sample is obtained from the subject at 1.5 to 2.5 hours post the administration of the IL1α inhibitor to the subject. In some embodiments, the second sample is obtained from the subject at about 1.5 hours post the administration. In some embodiments, the second sample is obtained from the subject at about 1.6 hours post the administration. In some embodiments, the second sample is obtained from the subject at about 1.7 hours post the administration. In some embodiments, the second sample is obtained from the subject at about 1.8 hours post the administration. In some embodiments, the second sample is obtained from the subject at about 1.9 hours post the administration. In some embodiments, the second sample is obtained from the subject at about 2.0 hours post the administration. In some embodiments, the second sample is obtained from the subject at about 2.1 hours post the administration. In some embodiments, the second sample is obtained from the subject at about 2.2 hours post the administration. In some embodiments, the second sample is obtained from the subject at about 2.3 hours post the administration. In some embodiments, the second sample is obtained from the subject at about 2.4 hours post the administration. In some embodiments, the second sample is obtained from the subject at about 2.5 hours post the administration.

In some embodiments, the methods provided herein further comprises adjusting treatment regimens (e.g., dosage, frequency, treatment cycle or treatment length) based on the measurements of the biomarkers provided herein.

The first and second samples may contain skin cells. In some embodiments, the sample is obtained by biopsy procedure and contain injured skin cells. In some embodiments, the size of the skin biopsy sample is about 3.5 mm to 4.5 mm. In some embodiments, the size of the skin biopsy sample is at least 4 mm. In some embodiments, the size of the skin biopsy sample is about 3.5 mm. In some embodiments, the size of the skin biopsy sample is about 3.6 mm. In some embodiments, the size of the skin biopsy sample is about 3.7 mm. In some embodiments, the size of the skin biopsy sample is about 3.8 mm. In some embodiments, the size of the skin biopsy sample is about 3.9 mm. In some embodiments, the size of the skin biopsy sample is about 4 mm. In some embodiments, the size of the skin biopsy sample is about 4.1 mm. In some embodiments, the size of the skin biopsy sample is about 4.2 mm. In some embodiments, the size of the skin biopsy sample is about 4.3 mm. In some embodiments, the size of the skin biopsy sample is about 4.4 mm. In some embodiments, the size of the skin biopsy sample is about 4.5 mm. In some embodiments, the size of the skin biopsy sample is about 4.6 mm. In some embodiments, the size of the skin biopsy sample is about 4.7 mm. In some embodiments, the size of the skin biopsy sample is about 4.8 mm. In some embodiments, the size of the skin biopsy sample is about 4.8 mm. In some embodiments, the size of the skin biopsy sample is about 4.9 mm. In some embodiments, the size of the skin biopsy sample is about 5.0 mm. In some embodiments, the biopsy sample obtained from the subject is cultured ex vivo.

In one embodiment, the size of the skin sample is at least 4.0 mm. In another embodiment, the size of the skin sample is about 4.0 mm.

In other embodiments, the sample used in the methods provided herein may comprises body fluids from a subject. In some embodiments, the sample is a blood sample. More detailed description of a sample is provided in Section 5.3 below.

In some embodiments, the levels of the one or more biomarkers provided herein can be measured at a time when IL1α would have been elevated should no inhibitor of IL1α be administrated (e.g., 4 to 24 hours post the biopsy procedure). In some embodiments, the levels of the one or more biomarkers are measured at least 4 hours post the biopsy procedure. In some embodiments, the levels of the one or more biomarkers are measured at least 5 hours post the biopsy procedure. In some embodiments, the levels of the one or more biomarkers are measured at least 6 hours post the biopsy procedure. In some embodiments, the levels of the one or more biomarkers are measured at least 7 hours post the biopsy procedure. In some embodiments, the levels of the one or more biomarkers are measured at least 8 hours post the biopsy procedure. In some embodiments, the levels of the one or more biomarkers are measured at least 9 hours post the biopsy procedure. In some embodiments, the levels of the one or more biomarkers are measured at least 10 hours post the biopsy procedure. In some embodiments, the levels of the one or more biomarkers are measured at least 11 hours post the biopsy procedure. In some embodiments, the levels of the one or more biomarkers are measured at least 12 hours post the biopsy procedure. In some embodiments, the levels of the one or more biomarkers are measured at least 13 hours post the biopsy procedure. In some embodiments, the levels of the one or more biomarkers are measured at least 14 hours post the biopsy procedure. In some embodiments, the levels of the one or more biomarkers are measured at least 15 hours post the biopsy procedure. In some embodiments, the levels of the one or more biomarkers are measured at least 16 hours post the biopsy procedure. In some embodiments, the levels of the one or more biomarkers are measured at least 17 hours post the biopsy procedure. In some embodiments, the levels of the one or more biomarkers are measured at least 18 hours post the biopsy procedure. In some embodiments, the levels of the one or more biomarkers are measured at least 19 hours post the biopsy procedure. In some embodiments, the levels of the one or more biomarkers are measured at least 20 hours post the biopsy procedure. In some embodiments, the levels of the one or more biomarkers are measured at least 21 hours post the biopsy procedure. In some embodiments, the levels of the one or more biomarkers are measured at least 22 hours post the biopsy procedure. In some embodiments, the levels of the one or more biomarkers are measured at least 23 hours post the biopsy procedure. In some embodiments, the levels of the one or more biomarkers are measured at least 24 hours post the biopsy procedure. In some embodiments, the levels of the one or more biomarkers are measured at least 25 hours post the biopsy procedure. In some embodiments, the levels of the one or more biomarkers are measured at least 30 hours post the biopsy procedure.

In one embodiment, the levels of the one or more biomarkers are measured at least 24 hours post the biopsy procedure.

In certain embodiments, the levels of one or more biomarkers are measured at least 24 hours post the biopsy procedure and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post the biopsy procedure. In another embodiment, the level of CXCL1 is measured at least 24 hours post the biopsy procedure. In another embodiment, the level of IL6 is measured at least 24 hours post the biopsy procedure.

In one embodiment, the levels of CSF3 (GCSF), CXCL1 and IL6 are measured at least 24 hours post the biopsy procedure.

In one embodiment, the levels of GCSF, CXCL1, IL6 and IL8 are measured at least 24 hours post the biopsy procedure.

In one embodiment, the levels of CXCL1, GCSF, GMCSF, IL6, IL8, MIP1a and TGFa are measured at least 24 hours post the biopsy procedure.

In one embodiment, the levels of GCSF, CXCL1, IL4, IL6, IL8, MDC and IP10 are measured at least 24 hours post the biopsy procedure.

In one embodiment, the levels of GCSF, CXCL1, IL4, IL6, IL8, MDC, IP10, GMCSF, MIP1a and TGFa are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, and PTGS2 are measured at least 24 hours post the biopsy procedure.

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post the biopsy procedure.

In one embodiment, the levels of CCL20, CSF3(GCSF), CXCL1, CXCL2, CXCL3, CXCL5, IL6, IL8, and PTGS2 are measured at least 24 hours post the biopsy procedure).

In yet another aspect, the levels of the biomarkers provided herein prior to treatment with an inhibitor of IL1α can be used for identifying a subject having a IL1α mediated disease who is likely to be responsive to a treatment comprising an inhibitor of IL1α or predicting the responsiveness of a subject having a IL1α mediated disease to a treatment comprising an inhibitor of IL1α. In some embodiments, the method comprises providing a sample from the subject; measuring levels of one or more biomarkers in the sample; identifying the subject with the IL1α mediated disease as responsive to the treatment with the inhibitor of IL1α when the levels of one or more biomarkers in the sample are higher than the reference levels of one or more biomarkers; and/or identifying the subject with the IL1α mediated disease as non-responsive to the treatment with the inhibitor of IL1α when the levels of one or more biomarkers in the sample are not higher than the reference levels of one or more biomarkers.

In some embodiments, the IL1α inhibitor is an anti-IL1α antibody, such as bermekimab.

In some embodiments, the IL1α mediated disease is atopic dermatitis, hidradenitis suppurativa, psoriasis, cutaneous lupus erythematosus, or autoimmune bullous disease. In one embodiment, the IL1α mediated disease is atopic dermatitis. In another embodiment, the IL1α mediated disease is hidradenitis suppurativa.

Thus, in some embodiments, the levels of the biomarkers provided herein prior to treatment with an anti-IL1α antibody (such as bermekimab) can be used for identifying a subject having atopic dermatitis who is likely to be responsive to a treatment comprising an anti-IL1α antibody (such as bermekimab) or predicting the responsiveness of a subject having atopic dermatitis to a treatment comprising an anti-IL1α antibody (such as bermekimab).

In other embodiments, the levels of the biomarkers provided herein prior to treatment with an anti-IL1α antibody (such as bermekimab) can be used for identifying a subject having hidradenitis suppurativa who is likely to be responsive to a treatment comprising an anti-IL1α antibody (such as bermekimab) or predicting the responsiveness of a subject having hidradenitis suppurativa to a treatment comprising an anti-IL1α antibody (such as bermekimab).

In some embodiments, the reference levels are the levels of the one or more biomarkers in a healthy subject. In other embodiments, the reference levels are the levels of the one or more biomarkers in an uninjured cell. In yet other embodiments, the reference levels are pre-determined levels.

In certain embodiments, the levels of one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one ore more biomarkers comprise CXCL1. In another embodiment, the one or more biomarkers comprise IL6.

In some embodiments, the method provided herein comprises measuring all of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the method provided herein comprises measuring all of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the method provided herein comprises measuring all of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. This approach may allow variations of individual gene expression in different subjects. Similarly, this method also applies to a subset including 2 or more biomarkers selected from CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In some embodiments, the one or more biomarkers are CSF3 (GCSF), CXCL1 and IL6. In some embodiments a composite score is calculated based on the levels of CSF3 (GCSF), CXCL1 and IL6, and wherein the method further comprises comparing the composite score to a reference score.

The levels of the biomarkers provided herein can be measured by determining protein levels or nucleic acid levels (e.g., mRNA, DNA or cDNA levels) of these biomarkers. In one embodiment, the levels of the biomarkers provided herein are measured by determining protein levels of these biomarkers. In another embodiment, the levels of the biomarkers provided herein are measured by determining mRNA levels of these biomarkers. Detailed description of these measurement methods are provided in the following sections.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In some embodiments, the method provided herein comprises measuring all of GCSF, CXCL1, IL4, IL6, IL8, MDC, IP10, GMCSF, MIP1a and TGFa. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. Similarly, this method also applies to a subset including 2 or more biomarkers selected from GCSF, CXCL1, IL4, IL6, IL8, MDC, IP10, GMCSF, MIP1a and TGFa.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In some embodiments, the one or more biomarkers are CSF3 (GCSF), CXCL1 and IL6. In some embodiments, a composite score is calculated based on the levels of CSF3 (GCSF), CXCL1 and IL6, and wherein the method further comprises comparing the composite score to a reference score.

In some embodiments, the levels of the biomarkers provided herein can be measured by determining protein levels of these biomarkers. Detailed description of these measurement methods are provided in the following sections.

In some embodiments of the various methods provided herein, the level(s) of the one or more biomarker(s) post treatment with the IL1α inhibitor (for example an anti-IL1α antibody, such as bermekimab) is compared with the reference level(s) of the same biomarker(s) from a sample cultured for the same amount of time without the treatment, and compared with another reference level(s) of the same biomarker(s) in a control sample. In some embodiments, the control sample is a positive control using another IL1α inhibitor (for example, the anti-IL1α antibody R&D, clone 4414). In other embodiments, the control sample is a negative control using a compound that is not an IL1α inhibitor. In yet other embodiments, another reference level(s) of the same biomarker(s) in a control sample is the level(s) of the biomarker(s) in a control sample just obtained from the subject, e.g., within 30 mins after obtaining the sample from the subject or within the period before IL1α level is elevated.

In other embodiments of the various methods provided herein, for each biomarker, four levels of the biomarker are obtained. Specifically, the first level of the biomarker is measured in a sample from a subject without treatment with the IL1α inhibitor (for example an anti-IL1α antibody, such as bermekimab) right after the sample is obtained from the subject (e.g., within 30 mins or before IL1α level is elevated); the second level of the biomarker is measured in a sample from a subject without treatment with the IL1α inhibitor (for example an anti-IL1α antibody, such as bermekimab) after the sample is cultured for a period of time (e.g., after IL1α level would usually elevate); the third level of the biomarker is measured in a sample from a subject with the treatment with the IL1α inhibitor (for example an anti-IL1α antibody, such as bermekimab) right after the sample is obtained from the subject (e.g., within 30 mins or before IL1α level would usually elevate); the fourth level of the biomarker is measured in a sample from a subject with the treatment with the IL1α inhibitor (for example an anti-IL1α antibody, such as bermekimab) after the sample is cultured for a period of time (e.g., after IL1α level would usually elevate without any treatment). In some embodiments, the first level and the second level are measured based on one sample but at different time points after being obtained from the subject. In some embodiments, the third level and the fourth level are measured based on one sample but at different time points after being obtained from the subject.

In some embodiments of various methods provided herein (including those described above), an inhibitor of IL1α provided herein is administered to a patient that has been determined likely to be responsive to the inhibitor of IL1α. In some embodiments, the inhibitor is an anti-IL1α antibody, such as bermekimab. So in one aspect, provided herein is a method of selectively treating a subject with a treatment comprising an inhibitor of IL1α, (for example an anti-IL1αantibody, such as bermekimab) comprising administering a therapeutically effective amount of the treatment comprising the inhibitor of IL1α(for example an anti-IL1α antibody, such as bermekimab) to the subject identified as being likely to be responsive to the treatment comprising the inhibitor of IL1α(for example an anti-IL1α antibody, such as bermekimab) according to the methods provided herein.

Also provided herein are methods of treating patients who have been previously treated but are non-responsive to standard therapies, as well as those who have not previously been treated. The disclosure also encompasses methods of treating patients regardless of patient's age, although some diseases or disorders are more common in certain age groups. The disclosure further encompasses methods of treating patients who have undergone surgery in an attempt to treat the disease or condition at issue, as well as those who have not. The treatment given to a patient may vary, depending on his/her prognosis. The skilled clinician will be able to readily determine specific secondary agents, types of surgery, and types of non-drug based standard therapy that can be effectively used to treat an individual patient.

In some embodiments, the subject has atopic dermatitis, hidradenitis suppurativa, psoriasis, cutaneous lupus erythematosus, or autoimmune bullous disease. In one embodiment, the subject has atopic dermatitis. In another embodiment, the subject has hidradenitis suppurativa.

In some embodiments, provided herein is a method for predicting or monitoring response to treatment of atopic dermatitis, hidradenitis suppurativa, psoriasis, cutaneous lupus erythematosus, or autoimmune bullous disease. In one embodiment, provided herein is a method for predicting or monitoring response to treatment of atopic dermatitis. In another embodiment, provided herein is a method for predicting or monitoring response to treatment of hidradenitis suppurativa.

In some embodiments, the method provided herein is for treating atopic dermatitis, hidradenitis suppurativa, psoriasis, cutaneous lupus erythematosus, or autoimmune bullous disease. In one embodiment, the method provided herein is for treating atopic dermatitis. In another embodiment, the method provided herein is for treating hidradenitis suppurativa.

5.2.3. Methods for Screening and Identifying IL1α Inhibitors

In another aspect, provided herein is a method for screening or identifying an agent that is capable of inhibiting IL1α using the biomarkers provided herein.

In some embodiments, the method comprises providing a sample; contacting the sample with the agent; measuring levels of one or more biomarkers in the sample; determining if the agent is capable of inhibiting IL1α based on the levels of the one or more biomarkers.

In some embodiments, the sample is obtained by biopsy procedure and contain injured skin cells. In some embodiments, the size of the skin biopsy sample is at least 4 mm. In some embodiments, the size of the skin biopsy sample is about 3.5 mm. In some embodiments, the size of the skin biopsy sample is about 3.6 mm. In some embodiments, the size of the skin biopsy sample is about 3.7 mm. In some embodiments, the size of the skin biopsy sample is about 3.8 mm. In some embodiments, the size of the skin biopsy sample is about 3.9 mm. In some embodiments, the size of the skin biopsy sample is about 4 mm. In some embodiments, the size of the skin biopsy sample is about 4.1 mm. In some embodiments, the size of the skin biopsy sample is about 4.2 mm. In some embodiments, the size of the skin biopsy sample is about 4.3 mm. In some embodiments, the size of the skin biopsy sample is about 4.4 mm. In some embodiments, the size of the skin biopsy sample is about 4.5 mm. In some embodiments, the size of the skin biopsy sample is about 4.6 mm. In some embodiments, the size of the skin biopsy sample is about 4.7 mm. In some embodiments, the size of the skin biopsy sample is about 4.8 mm. In some embodiments, the size of the skin biopsy sample is about 4.8 mm. In some embodiments, the size of the skin biopsy sample is about 4.9 mm. In some embodiments, the size of the skin biopsy sample is about 5.0 mm.

In one embodiment, the size of the skin sample is at least 4.0 mm. In another embodiment, the size of the skin sample is about 4.0 mm.

In some embodiments, the biopsy sample obtained from the subject is cultured ex vivo. The agent can be administered to the sample prior to the ex vivo culturing. In some embodiments, the agent can be administered to the sample during the ex vivo culturing. In other embodiments, the sample is cultured ex vivo for a period of time prior to administering to the sample the agent.

In some embodiments, the levels of the one or more biomarkers provided herein can be measured at a time when IL1α would have been elevated should no inhibitor of IL1α be administrated (e.g., 4 to 24 hours post the biopsy procedure).

In one embodiment the levels of the one or more biomarkers provided herein are measured in a skin sample at 4 hours post the biopsy procedure. In certain such embodiments, the levels of one or more biomarkers provided herein are measured in a skin sample that has a size of at least 4.0 mm at 4 hours post the biopsy procedure. In other embodiments, the levels of one or more biomarkers provided herein are measured in a skin sample that has a size of about 4.0 mm at 4 hours post the biopsy procedure.

In another embodiment, the levels of the one or more biomarkers provided herein are measured in a skin sample at 24 hours post the biopsy procedure. In certain such embodiments, the levels of one or more biomarkers provided herein are measured in a skin sample that has a size of at least 4.0 mm at 24 hours post the biopsy procedure. In other embodiments, the levels of one or more biomarkers provided herein are measured in a skin sample that has a size of about 4.0 mm at 24 hours post the biopsy procedure.

The levels of the one or more biomarkers can be measured at least 4 hours post administration of agent. In some embodiments, the levels of the one or more biomarkers are measured at least 5 hours post administration of the agent. In some embodiments, the levels of the one or more biomarkers are measured at least 6 hours post administration of the agent. In some embodiments, the levels of the one or more biomarkers are measured at least 7 hours post administration of the agent. In some embodiments, the levels of the one or more biomarkers are measured at least 8 hours post administration of the agent. In some embodiments, the levels of the one or more biomarkers are measured at least 9 hours post administration of the agent. In some embodiments, the levels of the one or more biomarkers are measured at least 10 hours post administration of the agent. In some embodiments, the levels of the one or more biomarkers are measured at least 11 hours post administration of the agent. In some embodiments, the levels of the one or more biomarkers are measured at least 12 hours post administration of the agent. In some embodiments, the levels of the one or more biomarkers are measured at least 13 hours post administration of the agent. In some embodiments, the levels of the one or more biomarkers are measured at least 14 hours post administration of the agent. In some embodiments, the levels of the one or more biomarkers are measured at least 15 hours post administration of the agent. In some embodiments, the levels of the one or more biomarkers are measured at least 16 hours post administration of the agent. In some embodiments, the levels of the one or more biomarkers are measured at least 17 hours post administration of the agent. In some embodiments, the levels of the one or more biomarkers are measured at least 18 hours post administration of the agent. In some embodiments, the levels of the one or more biomarkers are measured at least 19 hours post administration of the agent. In some embodiments, the levels of the one or more biomarkers are measured at least 20 hours post administration of the agent. In some embodiments, the levels of the one or more biomarkers are measured at least 21 hours post administration of the agent. In some embodiments, the levels of the one or more biomarkers are measured at least 22 hours post administration of the agent. In some embodiments, the levels of the one or more biomarkers are measured at least 23 hours post administration of the agent. In some embodiments, the levels of the one or more biomarkers are measured at least 24 hours post administration of the agent. In some embodiments, the levels of the one or more biomarkers are measured at least 25 hours post administration of the agent. In some embodiments, the levels of the one or more biomarkers are measured at least 30 hours post administration of the agent.

In one embodiment, the levels of the one or more biomarkers are measured at least 24 hours post administration of the inhibitor of IL1α(for example, an anti-IL1α antibody, such as bermekimab).

In certain embodiments, the levels of one or more biomarkers are measured at least 24 hours post administration of the inhibitor of IL1α and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post administration of the inhibitor of IL1α(for example, an anti-IL1α antibody, such as bermekimab). In another embodiment, the level of CXCL1 is measured at least 24 hours post administration of the inhibitor of IL1α(for example, an anti-IL1α antibody, such as bermekimab). In another embodiment, the level of IL6 is measured at least 24 hours post administration of the inhibitor of IL1α(for example, an anti-IL1α antibody, such as bermekimab).

In one embodiment, the levels of CSF3 (GCSF), CXCL1 and IL6 are measured at least 24 hours post administration of the inhibitor of IL1α(for example, an anti-IL1α antibody, such as bermekimab).

In one embodiment, the levels of GCSF, CXCL1, IL6 and IL8 are measured at least 24 hours post administration of the inhibitor of IL1α(for example, an anti-IL1α antibody, such as bermekimab).

In one embodiment, the levels of CXCL1, GCSF, GMCSF, IL6, IL8, MIP1a and TGFa are measured at least 24 hours post administration of the inhibitor of IL1α(for example, an anti-IL1α antibody, such as bermekimab).

In one embodiment, the levels of GCSF, CXCL1, IL4, IL6, IL8, MDC and IP10 are measured at least 24 hours post administration of the inhibitor of IL1α(for example, an anti-IL1α antibody, such as bermekimab).

In one embodiment, the levels of GCSF, CXCL1, IL4, IL6, IL8, MDC, IP10, GMCSF, MIP1a and TGFa are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post administration of the inhibitor of IL1α(for example, an anti-IL1α antibody, such as bermekimab).

In one embodiment, the levels of CCL20, CCL22, CSF3(GCSF), CXCL1, CXCL2, CXCL3, CXCL5, CXCL6, IL6, IL8, and PTGS2 are measured at least 24 hours post administration of the inhibitor of IL1α(for example, an anti-IL1α antibody, such as bermekimab).

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post administration of the inhibitor of IL1α(for example, an anti-IL1α antibody, such as bermekimab).

In one embodiment, the levels of CCL20, CSF3(GCSF), CXCL1, CXCL2, CXCL3, CXCL5, IL6, IL8, and PTGS2 are measured at least 24 hours post administration of the inhibitor of IL1α(for example, an anti-IL1α antibody, such as bermekimab).

In some embodiments, the levels of the one or more biomarkers are measured at least 4 hours post the biopsy procedure. In some embodiments, the levels of the one or more biomarkers are measured at least 5 hours post the biopsy procedure. In some embodiments, the levels of the one or more biomarkers are measured at least 6 hours post the biopsy procedure. In some embodiments, the levels of the one or more biomarkers are measured at least 7 hours post the biopsy procedure. In some embodiments, the levels of the one or more biomarkers are measured at least 8 hours post the biopsy procedure. In some embodiments, the levels of the one or more biomarkers are measured at least 9 hours post the biopsy procedure. In some embodiments, the levels of the one or more biomarkers are measured at least 10 hours post the biopsy procedure. In some embodiments, the levels of the one or more biomarkers are measured at least 11 hours post the biopsy procedure. In some embodiments, the levels of the one or more biomarkers are measured at least 12 hours post the biopsy procedure. In some embodiments, the levels of the one or more biomarkers are measured at least 13 hours post the biopsy procedure. In some embodiments, the levels of the one or more biomarkers are measured at least 14 hours post the biopsy procedure. In some embodiments, the levels of the one or more biomarkers are measured at least 15 hours post the biopsy procedure. In some embodiments, the levels of the one or more biomarkers are measured at least 16 hours post the biopsy procedure. In some embodiments, the levels of the one or more biomarkers are measured at least 17 hours post the biopsy procedure. In some embodiments, the levels of the one or more biomarkers are measured at least 18 hours post the biopsy procedure. In some embodiments, the levels of the one or more biomarkers are measured at least 19 hours post the biopsy procedure. In some embodiments, the levels of the one or more biomarkers are measured at least 20 hours post the biopsy procedure. In some embodiments, the levels of the one or more biomarkers are measured at least 21 hours post the biopsy procedure. In some embodiments, the levels of the one or more biomarkers are measured at least 22 hours post the biopsy procedure. In some embodiments, the levels of the one or more biomarkers are measured at least 23 hours post the biopsy procedure. In some embodiments, the levels of the one or more biomarkers are measured at least 24 hours post the biopsy procedure. In some embodiments, the levels of the one or more biomarkers are measured at least 25 hours post the biopsy procedure. In some embodiments, the levels of the one or more biomarkers are measured at least 30 hours post the biopsy procedure.

In one embodiment, the levels of the one or more biomarkers are measured at least 24 hours post the biopsy procedure.

In certain embodiments, the levels of one or more biomarkers are measured at least 24 hours post the biopsy procedure and the one or more biomarkers are selected from the group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the level of CSF3 (GCSF) is measured at least 24 hours post the biopsy procedure. In another embodiment, the level of CXCL1 is measured at least 24 hours post the biopsy procedure. In another embodiment, the level of IL6 is measured at least 24 hours post the biopsy procedure.

In one embodiment, the levels of CSF3 (GCSF), CXCL1 and IL6 are measured at least 24 hours post the biopsy procedure.

In one embodiment, the levels of GCSF, CXCL1, IL6 and IL8 are measured at least 24 hours post the biopsy procedure.

In one embodiment, the levels of CXCL1, GCSF, GMCSF, IL6, IL8, MIP1a and TGFa are measured at least 24 hours post the biopsy procedure.

In one embodiment, the levels of GCSF, CXCL1, IL4, IL6, IL8, MDC and IP10 are measured at least 24 hours post the biopsy procedure.

In one embodiment, the levels of GCSF, CXCL1, IL4, IL6, IL8, MDC, IP10, GMCSF, MIP1a and TGFa are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3 are measured at least 24 hours post the biopsy procedure.

In one embodiment, the levels of CCL20, CCL22, CSF3(GCSF), CXCL1, CXCL2, CXCL3, CXCL5, CXCL6, IL6, IL8, and PTGS2 are measured at least 24 hours post the biopsy procedure.

In one embodiment, the levels of CCL20, CCL3, CCL4, CCL8, CFB, CSF3(GCSF), CXCL1, CXCL2, CXCL3, CXCL5, IL1B, IL6, IL8, MMP3 are measured at least 24 hours post the biopsy procedure.

In one embodiment, the levels of CCL20, CSF3(GCSF), CXCL1, CXCL2, CXCL3, CXCL5, IL6, IL8, and PTGS2 are measured at least 24 hours post the biopsy procedure.

The effects of the agent can be determined by comparing the levels of the one or more biomarkers provided herein with reference levels of these biomarkers.

The reference levels of these biomarkers can be the levels of these biomarkers in a reference injured skin sample (for example, a skin biopsy sample) without any influence by an inhibitor of IL1α(for example an anti-IL1α antibody, such as bermekimab), and the levels of the biomarkers in the sample and the levels of the biomarkers in the reference sample are measured at the same time point (for example, at least 4 hours or at least 24 hours post the biopsy procedure).

In some embodiments, the reference levels of the one or more biomarkers are the levels of the one or more biomarkers in a reference sample from the subject prior to administration of an inhibitor of IL1α. In some embodiments, the reference levels of the one or more biomarkers are the levels of the one or more biomarkers in a reference sample from the subject without administration of an inhibitor of IL1α. In some embodiments, the reference levels of the one or more biomarkers are pre-determined levels of the one or more biomarkers. In some embodiments, the reference levels of the one or more biomarkers are the levels of the one or more biomarkers in a reference sample administered with a control agent (e.g., a positive control agent that inhibits IL1α, such as the anti-IL1α antibody R&D, clone 4414, or a negative control agent that does not inhibit IL1α).

In one embodiment, the reference levels of the one or more biomarkers are the levels of the one or more biomarkers in a reference skin biopsy sample from the subject without administration of the inhibitor of IL1α, and the levels of the biomarkers in the sample and the levels of the biomarkers in the reference sample are measured at the same time point.

When the levels of the biomarkers are compared to the reference levels that represent the elevated levels of these biomarkers post injury (e.g. biopsy induced injury) but without the influence of an inhibitor of IL1α, the lower levels of the one or more biomarkers as compared with the references indicate that the agent is capable of inhibiting IL1α.

In some embodiments, the agent is identified as capable of inhibiting IL1α if the levels of the one or more biomarkers in the sample are at least 20%, at least 30%, at least 35%, at least 40%, or at least 50% less than the reference levels.

In one embodiment, the agent is identified as capable of inhibiting IL1α if the levels of the one or more cytokine biomarkers secreted by the sample (for example, as measured by a Luminex assay) are at least 50% less than the reference levels. In another embodiment, the agent is identified as capable of inhibiting IL1α if the mRNA levels (for example, as measured by a Nanostring assay) of the one or more biomarkers in the sample are at least 30% less than the reference levels. In another embodiment, the agent is identified as capable of inhibiting IL1α if the mRNA levels (for example, as measured by an RNAseq assay) of the one or more biomarkers in the sample are at least 20% less than the reference levels.

In some embodiments, the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2. In other embodiments, the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the one or more biomarkers comprises CCL20. In some embodiments, the one or more biomarkers comprises CCL22. In some embodiments, the one or more biomarkers comprises CSF3. In other embodiments, the one or more biomarkers comprises CXCL1. In other embodiments, the one or more biomarkers comprises CXCL2. In other embodiments, the one or more biomarkers comprises CXCL3. In other embodiments, the one or more biomarkers comprises CXCL5. In other embodiments, the one or more biomarkers comprises CXCL6. In yet other embodiments, the one or more biomarkers comprises IL6. In yet other embodiments, the one or more biomarkers comprises IL8. In yet other embodiments, the one or more biomarkers comprises PTGS2. In yet other embodiments, the one or more biomarkers comprises CCL3. In yet other embodiments, the one or more biomarkers comprises CCL4. In yet other embodiments, the one or more biomarkers comprises CCL8. In yet other embodiments, the one or more biomarkers comprises CFB. In yet other embodiments, the one or more biomarkers comprises IL1B. In yet other embodiments, the one or more biomarkers comprises MMP3.

In one embodiment, the one or more biomarkers comprise CSF3 (GCSF). In another embodiment, the one or more biomarkers comprise CXCL1. In another embodiment, the one or more biomarkers comprise IL6.

In some embodiments, the method provided herein comprises measuring all of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the method provided herein comprises measuring all of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the method provided herein comprises measuring all of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. This approach may allow variations of individual gene expression in different subjects. Similarly, this method also applies to a subset including 2 or more biomarkers selected from CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3

In some embodiments, the one or more biomarkers are CCL20, CCL22, CSF3(GCSF), CXCL1, CXCL2, CXCL3, CXCL5, CXCL6, IL6, IL8, and PTGS2. In some embodiments, a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

In other embodiments, the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2. In some embodiments, a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

In other embodiments, the one or more biomarkers are CCL20, CSF3(GCSF), CXCL1, CXCL2, CXCL3, CXCL5, IL6, IL8, and PTGS2. In some embodiments, a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

In other embodiments, the one or more biomarkers are CSF3 (GCSF), CXCL1 and IL6. In some embodiments, a composite score is calculated based on the levels of CSF3 (GCSF), CXCL1 and IL6, and wherein the method further comprises comparing the composite score to a reference score.

The levels of the biomarkers provided herein can be measured by determining protein levels or nucleic acid levels (e.g., mRNA, DNA or cDNA levels) of these biomarkers. In one embodiment, the levels of the biomarkers provided herein are measured by determining protein levels of these biomarkers. In another embodiment, the levels of the biomarkers provided herein are measured by determining mRNA levels of these biomarkers. Detailed description of these measurement methods are provided in the following sections.

In some embodiments, the levels of the one or more biomarkers are the levels of cytokines secreted by the sample selected from a group consisting of GCSF, CXCL1, IL4, IL6, IL8, MDC, IP10, GMCSF, MIP1a and TGFa.

In some embodiments, the levels of the one or more biomarkers are the levels of cytokines secreted by the sample selected from a group consisting of GCSF, CXCL1, IL4, IL6, IL8, MDC and IP10.

In some embodiments, the levels of the one or more biomarkers are the levels of cytokines secreted by the sample selected from a group consisting of CXCL1, GCSF, GMCSF, IL6, IL8, MIP1a and TGFa.

In some embodiments, the one or more biomarkers comprises GCSF. In some embodiments, the one or more biomarkers comprises CXCL1. In other embodiments, the one or more biomarkers comprises IL4. In other embodiments, the one or more biomarkers comprises IL6. In other embodiments, the one or more biomarkers comprises IL8. In other embodiments, the one or more biomarkers comprises MDC. In other embodiments, the one or more biomarkers comprises IP10. In other embodiments, the one or more biomarkers comprises GMCSF. In other embodiments, the one or more biomarkers comprises MIP1a. In other embodiments, the one or more biomarkers comprises TGFa.

In some embodiments, the method provided herein comprises measuring all of GCSF, CXCL1, IL4, IL6, IL8, MDC, IP10, GMCSF, MIP1a and TGFa. Totality of the expression levels of these biomarkers is used to determine the effects of the inhibitor. Any classification methods or algorithms useful for comparing the totality of the expressions of these biomarkers with a reference are included herein. For example, a composite score based on the expression levels of these biomarkers can be used. In some instances a composite score may be calculated based on the geometric mean of the expression levels of these biomarkers. A composite score based on comparing the expression levels of these biomarkers with and without treatment may be used. In some instances, a composite score may be calculated using the average of percentage of reduction on the expression levels (following treatment) of these biomarkers. Similarly, this method also applies to a subset including 2 or more biomarkers selected from GCSF, CXCL1, IL4, IL6, IL8, MDC, IP10, GMCSF, MIP1a and TGFa.

In some embodiments, the one or more biomarkers are GCSF, CXCL1, IL4, IL6, IL8, MDC and IP10. In some embodiments, a composite score is calculated based on the levels of GCSF, CXCL1, IL4, IL6, IL8, MDC and IP10, and wherein the method further comprises comparing the composite score to a reference score.

In some embodiments, the one or more biomarkers are CXCL1, GCSF, GMCSF, IL6, IL8, MIP1a and TGFa. In some embodiments, a composite score is calculated based on the levels of CXCL1, GCSF, GMCSF, IL6, IL8, MIP1a and TGFa, and wherein the method further comprises comparing the composite score to a reference score.

In some embodiments, the one or more biomarkers are GCSF, CXCL1, IL6 and IL8. In some embodiments, a composite score is calculated based on the levels of GCSF, CXCL1, IL6 and IL8, and wherein the method further comprises comparing the composite score to a reference score.

In some embodiments, the one or more biomarkers are CSF3 (GCSF), CXCL1 and IL6. In some embodiments, a composite score is calculated based on the levels of CSF3 (GCSF), CXCL1 and IL6, and wherein the method further comprises comparing the composite score to a reference score.

In some embodiments, the levels of the biomarkers provided herein can be measured by determining protein levels of these biomarkers. Detailed description of these measurement methods are provided in the following sections.

The agent identified as capable of inhibiting IL1α can be subject to further validation tests, e.g., to evaluate its biological or other properties.

In another aspect, provided herein is a composition comprising an agent identified according to the methods provided herein and use of this composition for treating a disease or disorder. In some embodiments, the disease or disorder is selected from the group consisting of atopic dermatitis, hidradenitis suppurativa, psoriasis, cutaneous lupus erythematosus, or autoimmune bullous disease.

In one embodiment, the disease or disorder is atopic dermatitis. In another embodiment, the disease or disorder is hidradenitis suppurativa.

### 5.3. Subjects, Samples, and Types of Cells

In certain embodiments, the various methods provided herein use samples (e.g., biological samples) from subjects or individuals (e.g., patients). The subject can be a healthy subject. The subject can be a patient. The subject can be a mammal, for example, a human. The subject can be male or female, and can be an adult, a child, or an infant. In certain embodiments, more than one sample from a subject can be obtained.

In some embodiments, the sample used in the present methods comprises a biopsy (e.g., a skin biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In some embodiments, the biopsy sample is about 3 mm to 20 mm. In some embodiments, the biopsy sample is about 3 mm to 10 mm. In other embodiments, the biopsy sample is 3 mm to 5 mm. In a specific embodiment, the biopsy sample is a skin biopsy sample of about 4 mm.

In certain embodiments, the sample used in the methods provided herein comprises body fluids from a subject. Non-limiting examples of body fluids include blood (e.g., whole blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids (including cerebrospinal fluid surrounding the brain and the spinal cord), synovial fluid, intracellular fluid (the fluid inside cells), and vitreous humour (the fluid in the eyeball). In some embodiments, the sample is a blood sample. The blood sample can be obtained using conventional techniques as described in, e.g., Innis et al, eds., PCR Protocols (Academic Press, 1990). White blood cells can be separated from blood samples using conventional techniques or commercially available kits, e.g., RosetteSep kit (Stein Cell Technologies, Vancouver, Canada). Sub-populations of white blood cells, e.g., mononuclear cells, B cells, T cells, monocytes, granulocytes, or lymphocytes, can be further isolated using conventional techniques, e.g., magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 mL.

In one embodiment, the sample used in the methods provided herein is obtained from the subject prior to the subject receiving a treatment. In another embodiment, the sample is obtained from the subject during the subject receiving a treatment. In another embodiment, the sample is obtained from the subject after the subject receiving a treatment. In various embodiments, the treatment comprises administering a compound (e.g., an IL1α inhibitor, such as anti-IL1α antibody, for instance bermekimab) to the subject.

In certain embodiments, the sample used in the methods provided herein comprises a plurality of cells, such as skin cells.

In certain embodiments, the number of cells used in the methods provided herein can range from a single cell to about 10⁹ cells. In some embodiments, the number of cells used in the methods provided herein is about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, or about 1×10⁹.

The number and type of cells collected from a subject can be monitored, for example, by measuring changes in cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examining the morphology of cells using light or confocal microscopy, and/or by measuring changes in gene expression using techniques well known in the art, such as PCR and gene expression profiling. These techniques can be used, too, to identify cells that are positive for one or more particular markers.

In certain embodiments, subsets of cells are used in the methods provided herein. Methods of sorting and isolating specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc. FACS is a well-known method for separating particles, including cells, based on the fluorescent properties of the particles (Kamarch, Methods Enzymol. 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one embodiment, RNA (e.g., mRNA) or protein is purified from a population of cells, and the level of a gene set is measured by mRNA or protein expression analysis. In certain embodiments, the level of a gene set is measured by transcriptomic profiling, qRT-PCR, microarray, high throughput sequencing, or other similar methods known in the art. In other embodiments, the level of a gene set is measured by ELISA, flow cytometry, immunofluorescence, or other similar methods known in the art and described in more detail below.

In some embodiments of various methods provided herein, a subject is a healthy subject. In other embodiments, the subject is a patient, e.g., having atopic dermatitis, hidradenitis suppurativa, psoriasis, cutaneous lupus erythematosus, or autoimmune bullous disease. In one embodiment, the patient is a subject having atopic dermatitis. In another embodiment, the patient is a subject having hidradenitis suppurativa.

### 5.4. Methods of Detecting and Quantifying Biomarkers

In certain embodiments, the biomarkers provide herein can be measured by the protein level, RNA level, DNA level, or cDNA level of the biomarker. In one embodiment, the biomarkers provided herein can measured by the protein level of these biomarkers. In another embodiment, the biomarkers provided herein can be measured by the mRNA levels of these biomarkers.

In certain embodiments of the various methods provided herein, the two or more of the steps are performed sequentially. In other embodiments of the methods provided herein, two or more of steps are performed in parallel (e.g., at the same time).

Several protein detection and quantization methods can be used to measure the level of a biomarker. Any suitable protein quantization method can be used. In some embodiments, antibody-based methods are used. Exemplary methods that can be used include, but are not limited to, immunoblotting (Western blot), ELISA, immunohistochemistry, flow cytometry, cytometry bead array, mass spectroscopy, and the like. Several types of ELISA are commonly used, including direct ELISA, indirect ELISA, and sandwich ELISA.

In some embodiments, the methods provided herein for detecting and quantifying the protein levels of one or more biomarkers are antibody based methods.

In certain embodiments, provided herein are methods of detecting and quantifying the protein level of biomarker (e.g., a gene product) from a biological sample, comprising contacting proteins within the sample with a first antibody that binds to the biomarker protein. In some embodiments, the methods provided herein further comprise (i) contacting the biomarker protein bound to the first antibody with a second antibody with a detectable label, wherein the second antibody binds to the biomarker protein, and wherein the second antibody binds to a different epitope on the biomarker protein than the first antibody; (ii) detecting the presence of the second antibody bound to the biomarker protein; and (iii) determining the amount of the biomarker protein based on the amount of detectable label in the second antibody. In other embodiments, the methods provided herein further comprise (i) contacting the biomarker protein bound to the first antibody with a second antibody with a detectable label, wherein the second antibody binds to the first antibody; (ii) detecting the presence of the second antibody bound to the first antibody; and (iii) determining the amount of the biomarker protein based on the amount of detectable label in the second antibody.

In some embodiments of the various methods provided herein, the method comprises using dual staining immunohistochemistry to determine the level of a biomarker. In a dual staining immunohistochemistry assay, a biomarker provided herein and another biomarker are simultaneously detected using a first labeled antibody targeting a biomarker provided herein and a second labeled antibody targeting the other biomarker. Such assay can improve the specificity, accuracy, and sensitivity for detecting and measuring a biomarker provided herein.

Thus, in some embodiments, the method provided herein comprises (i) contacting proteins within a sample with a first antibody that binds to a biomarker provided herein, the first antibody being coupled with a first detectable label; (ii) contacting the proteins within the sample with a second antibody that binds to another biomarker, the second antibody being coupled with a second detectable label; (iii) detecting the presence of the first antibody and the second antibody bound to the proteins; and (iv) determining the level of the biomarker provided herein based on the amount of detectable label in the first antibody, and determining the level of the other biomarker based on the amount of detectable label in the second antibody.

In some embodiments, the methods provided herein comprise measuring the protein level of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and/or MMP3. In other embodiments, the methods provided herein comprise measuring the protein level of GCSF, CXCL1, IL4, IL6, IL8, MDC, IP10, GMCSF, MIP1a and/or TGFa.

In some embodiments, the methods provided herein comprise measuring the protein level of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the protein level of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the protein level of CCL20. In some embodiments, the methods provided herein comprise measuring the protein level of CCL22. In some embodiments, the methods provided herein comprise measuring the protein level of CSF3. In some embodiments, the methods provided herein comprise measuring the protein level of CXCL1. In some embodiments, the methods provided herein comprise measuring the protein level of CXCL2. In some embodiments, the methods provided herein comprise measuring the protein level of CXCL3. In some embodiments, the methods provided herein comprise measuring the protein level of CXCL5. In some embodiments, the methods provided herein comprise measuring the protein level of CXCL6. In some embodiments, the methods provided herein comprise measuring the protein level of IL6. In some embodiments, the methods provided herein comprise measuring the protein level of IL8. In some embodiments, the methods provided herein comprise measuring the protein level of PTGS2. In some embodiments, the methods provided herein comprise measuring the protein level of CCL3. In some embodiments, the methods provided herein comprise measuring the protein level of CCL4. In some embodiments, the methods provided herein comprise measuring the protein level of CCL8. In some embodiments, the methods provided herein comprise measuring the protein level of CFB. In some embodiments, the methods provided herein comprise measuring the protein level of IL1B. In some embodiments, the methods provided herein comprise measuring the protein level of MMP3. In some embodiments, the methods provided herein comprise measuring the protein level of GCSF. In some embodiments, the methods provided herein comprise measuring the protein level of CXCL1. In some embodiments, the methods provided herein comprise measuring the protein level of IL4. In some embodiments, the methods provided herein comprise measuring the protein level of IL6. In some embodiments, the methods provided herein comprise measuring the protein level of IL8. In some embodiments, the methods provided herein comprise measuring the protein level of MDC. In some embodiments, the methods provided herein comprise measuring the protein level of IP10. In some embodiments, the methods provided herein comprise measuring the protein level of GMCSF. In some embodiments, the methods provided herein comprise measuring the protein level of MIP1a. In some embodiments, the methods provided herein comprise measuring the protein level of TGFa.

In one embodiment, the methods provided herein comprise measuring the protein level of CSF3 (GCSF). In another embodiment, the methods provided herein comprise measuring the protein level of CXCL1. In another embodiment, the methods provided herein comprise measuring the protein level of IL6.

In some embodiments, the methods provided herein comprise measuring the protein levels of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the protein levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the protein levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the methods provided herein comprise measuring the protein levels of CCL20, CCL22, CSF3(GCSF), CXCL1, CXCL2, CXCL3, CXCL5, CXCL6, IL6, IL8, and PTGS2.

In some embodiments, the methods provided herein comprise measuring the protein levels of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the methods provided herein comprise measuring the protein levels of CCL20, CSF3(GCSF), CXCL1, CXCL2, CXCL3, CXCL5, IL6, IL8, and PTGS2.

In some embodiments, the methods provided herein comprise measuring the protein levels of CSF3 (GCSF), CXCL1 and IL6.

In some embodiments, the methods provided herein comprise measuring the protein levels of GCSF, CXCL1, IL4, IL6, IL8, MDC, IP10, GMCSF, MIP1a and TGFa.

In some embodiments, the methods provided herein comprise measuring the protein levels of GCSF, CXCL1, IL4, IL6, IL8, MDC and IP10.

In some embodiments, the methods provided herein comprise measuring the protein levels of CXCL1, GCSF, GMCSF, IL6, IL8, MIP1a and TGFa.

In some embodiments, the methods provided herein comprise measuring the protein levels of GCSF, CXCL1, IL6 and IL8.

Several methods of detecting or quantitating mRNA levels are known in the art. Exemplary methods include, but are not limited to, northern blots, ribonuclease protection assays, PCR-based methods, a Nanostring assay, and the like. The mRNA sequence of a biomarker can be used to prepare a probe that is at least partially complementary to the mRNA sequence. The probe can then be used to detect the mRNA in a sample, using any suitable assay, such as PCR-based methods, northern blotting, a dipstick assay, and the like.

In other embodiments, a nucleic acid assay for testing for compound activity in a biological sample can be prepared. An assay typically contains a solid support and at least one nucleic acid contacting the support, where the nucleic acid corresponds to at least a portion of an mRNA that has altered expression during a compound treatment in a patient, such as the mRNA of a biomarker. The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5-carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

In some embodiments, the mRNA sequences comprise at least one mRNA of a biomarker provided herein.

The nucleic acids may be present in specific, addressable locations on a solid support, each corresponding to at least a portion of mRNA sequences that are differentially expressed upon treatment of a compound in a cell or a patient.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science 1992, 258:818-821 and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect the expression of a biomarker provided herein. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

Other methods, such as RNA sequencing-based methods, can also be used to detect the expression of a biomarker provided herein. In certain embodiments, the RNA expression profile of biomarkers is measured by high-throughput sequencing (e.g., whole transcriptome shotgun sequencing (RNA sequencing or RNAseq)). RNA sequencing methods have been described elsewhere (see Wang Z, Gerstein M and Snyder M, Nature Review Genetics (2009) 10: 57-63; Maher C A et al., Nature (2009) 458: 97-101; Kukrba K & Montgomery S B, Cold Spring Harbor Protocols (2015) 2015 (11): 951-969).

In some embodiments, the methods provided herein comprise measuring the mRNA level of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and/or MMP3. In other embodiments, the methods provided herein comprise measuring the mRNA level of GCSF, CXCL1, IL4, IL6, IL8, MDC, IP10, GMCSF, MIP1a and/or TGFa.

In some embodiments, the methods provided herein comprise measuring the mRNA level of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the mRNA level of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the mRNA level of CCL20. In some embodiments, the methods provided herein comprise measuring the mRNA level of CCL22. In some embodiments, the methods provided herein comprise measuring the mRNA level of CSF3. In some embodiments, the methods provided herein comprise measuring the mRNA level of CXCL1. In some embodiments, the methods provided herein comprise measuring the mRNA level of CXCL2. In some embodiments, the methods provided herein comprise measuring the mRNA level of CXCL3. In some embodiments, the methods provided herein comprise measuring the mRNA level of CXCL5. In some embodiments, the methods provided herein comprise measuring the mRNA level of CXCL6. In some embodiments, the methods provided herein comprise measuring the mRNA level of IL6. In some embodiments, the methods provided herein comprise measuring the mRNA level of IL8. In some embodiments, the methods provided herein comprise measuring the mRNA level of PTGS2. In some embodiments, the methods provided herein comprise measuring the mRNA level of CCL3. In some embodiments, the methods provided herein comprise measuring the mRNA level of CCL4. In some embodiments, the methods provided herein comprise measuring the mRNA level of CCL8. In some embodiments, the methods provided herein comprise measuring the mRNA level of CFB. In some embodiments, the methods provided herein comprise measuring the mRNA level of IL1B. In some embodiments, the methods provided herein comprise measuring the mRNA level of MMP3. In some embodiments, the methods provided herein comprise measuring the mRNA level of GCSF. In some embodiments, the methods provided herein comprise measuring the mRNA level of CXCL1. In some embodiments, the methods provided herein comprise measuring the mRNA level of IL4. In some embodiments, the methods provided herein comprise measuring the mRNA level of IL6. In some embodiments, the methods provided herein comprise measuring the mRNA level of IL8. In some embodiments, the methods provided herein comprise measuring the mRNA level of MDC. In some embodiments, the methods provided herein comprise measuring the mRNA level of IP10. In some embodiments, the methods provided herein comprise measuring the mRNA level of GMCSF. In some embodiments, the methods provided herein comprise measuring the mRNA level of MIP1a. In some embodiments, the methods provided herein comprise measuring the mRNA level of TGFa.

In one embodiment, the methods provided herein comprise measuring the mRNA level of CSF3 (GCSF). In another embodiment, the methods provided herein comprise measuring the mRNA level of CXCL1. In another embodiment, the methods provided herein comprise measuring the mRNA level of IL6.

In some embodiments, the methods provided herein comprise measuring the mRNA levels of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the mRNA levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the mRNA levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the methods provided herein comprise measuring the mRNA levels of CCL20, CCL22, CSF3(GCSF), CXCL1, CXCL2, CXCL3, CXCL5, CXCL6, IL6, IL8, and PTGS2.

In some embodiments, the methods provided herein comprise measuring the mRNA levels of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the methods provided herein comprise measuring the mRNA levels of CCL20, CSF3(GCSF), CXCL1, CXCL2, CXCL3, CXCL5, IL6, IL8, and PTGS2.

In some embodiments, the methods provided herein comprise measuring the mRNA levels of CSF3 (GCSF), CXCL1 and IL6.

In some embodiments, the methods provided herein comprise measuring the mRNA levels of GCSF, CXCL1, IL4, IL6, IL8, MDC, IP10, GMCSF, MIP1a and TGFa.

In some embodiments, the methods provided herein comprise measuring the mRNA levels of GCSF, CXCL1, IL4, IL6, IL8, MDC and IP10.

In some embodiments, the methods provided herein comprise measuring the mRNA levels of CXCL1, GCSF, GMCSF, IL6, IL8, MIP1a and TGFa.

In some embodiments, the methods provided herein comprise measuring the mRNA levels of GCSF, CXCL1, IL6 and IL8.

In some embodiments, the methods provided herein comprise measuring the cDNA level of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and/or MMP3. In other embodiments, the methods provided herein comprise measuring the cDNA level of GCSF, CXCL1, IL4, IL6, IL8, MDC, IP10, GMCSF, MIP1a and/or TGFa.

In some embodiments, the methods provided herein comprise measuring the cDNA level of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the cDNA level of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the cDNA level of CCL20. In some embodiments, the methods provided herein comprise measuring the cDNA level of CCL22. In some embodiments, the methods provided herein comprise measuring the cDNA level of CSF3. In some embodiments, the methods provided herein comprise measuring the cDNA level of CXCL1. In some embodiments, the methods provided herein comprise measuring the cDNA level of CXCL2. In some embodiments, the methods provided herein comprise measuring the cDNA level of CXCL3. In some embodiments, the methods provided herein comprise measuring the cDNA level of CXCL5. In some embodiments, the methods provided herein comprise measuring the cDNA level of CXCL6. In some embodiments, the methods provided herein comprise measuring the cDNA level of IL6. In some embodiments, the methods provided herein comprise measuring the cDNA level of IL8. In some embodiments, the methods provided herein comprise measuring the cDNA level of PTGS2. In some embodiments, the methods provided herein comprise measuring the cDNA level of CCL3. In some embodiments, the methods provided herein comprise measuring the cDNA level of CCL4. In some embodiments, the methods provided herein comprise measuring the cDNA level of CCL8. In some embodiments, the methods provided herein comprise measuring the cDNA level of CFB. In some embodiments, the methods provided herein comprise measuring the cDNA level of IL1B. In some embodiments, the methods provided herein comprise measuring the cDNA level of MMP3. In some embodiments, the methods provided herein comprise measuring the cDNA level of GCSF. In some embodiments, the methods provided herein comprise measuring the cDNA level of CXCL1. In some embodiments, the methods provided herein comprise measuring the cDNA level of IL4. In some embodiments, the methods provided herein comprise measuring the cDNA level of IL6. In some embodiments, the methods provided herein comprise measuring the cDNA level of IL8. In some embodiments, the methods provided herein comprise measuring the cDNA level of MDC. In some embodiments, the methods provided herein comprise measuring the cDNA level of IP10. In some embodiments, the methods provided herein comprise measuring the cDNA level of GMCSF. In some embodiments, the methods provided herein comprise measuring the cDNA level of MIP1a. In some embodiments, the methods provided herein comprise measuring the cDNA level of TGFa.

In one embodiment, the methods provided herein comprise measuring the cDNA level of CSF3 (GCSF). In another embodiment, the methods provided herein comprise measuring the cDNA level of CXCL1. In another embodiment, the methods provided herein comprise measuring the cDNA level of IL6.

In some embodiments, the methods provided herein comprise measuring the cDNA levels of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the cDNA levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the methods provided herein comprise measuring the cDNA levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In some embodiments, the methods provided herein comprise measuring the cDNA levels of CCL20, CCL22, CSF3(GCSF), CXCL1, CXCL2, CXCL3, CXCL5, CXCL6, IL6, IL8, and PTGS2.

In some embodiments, the methods provided herein comprise measuring the cDNA levels of CCL20, CCL3, CCL4, CCL8, CFB, CSF3(GCSF), CXCL1, CXCL2, CXCL3, CXCL5, IL1B, IL6, IL8, MMP3, and PTGS2.

In some embodiments, the methods provided herein comprise measuring the cDNA levels of CCL20, CSF3(GCSF), CXCL1, CXCL2, CXCL3, CXCL5, IL6, IL8, and PTGS2.

In some embodiments, the methods provided herein comprise measuring the cDNA levels of CSF3 (GCSF), CXCL1 and IL6.

In some embodiments, the methods provided herein comprise measuring the cDNA levels of GCSF, CXCL1, IL4, IL6, IL8, MDC, IP10, GMCSF, MIP1a and TGFa.

In some embodiments, the methods provided herein comprise measuring the cDNA levels of GCSF, CXCL1, IL4, IL6, IL8, MDC and IP10.

In some embodiments, the methods provided herein comprise measuring the cDNA levels of CXCL1, GCSF, GMCSF, IL6, IL8, MIP1a and TGFa.

In some embodiments, the methods provided herein comprise measuring the cDNA levels of GCSF, CXCL1, IL6 and IL8.

In some embodiments, the methods provided herein comprise measuring the DNA level of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and/or MMP3. In other embodiments, the methods provided herein comprise measuring the DNA level of GCSF, CXCL1, IL4, IL6, IL8, MDC, IP10, GMCSF, MIP1a and/or TGFa. In some embodiments, the methods provided herein comprise measuring the DNA level of CCL20. In some embodiments, the methods provided herein comprise measuring the DNA level of CCL22. In some embodiments, the methods provided herein comprise measuring the DNA level of CSF3. In some embodiments, the methods provided herein comprise measuring the DNA level of CXCL1. In some embodiments, the methods provided herein comprise measuring the DNA level of CXCL2. In some embodiments, the methods provided herein comprise measuring the DNA level of CXCL3. In some embodiments, the methods provided herein comprise measuring the DNA level of CXCL5. In some embodiments, the methods provided herein comprise measuring the DNA level of CXCL6. In some embodiments, the methods provided herein comprise measuring the DNA level of IL6. In some embodiments, the methods provided herein comprise measuring the DNA level of IL8. In some embodiments, the methods provided herein comprise measuring the DNA level of PTGS2. In some embodiments, the methods provided herein comprise measuring the DNA level of CCL3. In some embodiments, the methods provided herein comprise measuring the DNA level of CCL4. In some embodiments, the methods provided herein comprise measuring the DNA level of CCL8. In some embodiments, the methods provided herein comprise measuring the DNA level of CFB. In some embodiments, the methods provided herein comprise measuring the DNA level of IL1B. In some embodiments, the methods provided herein comprise measuring the DNA level of MMP3. In some embodiments, the methods provided herein comprise measuring the DNA level of GCSF. In some embodiments, the methods provided herein comprise measuring the DNA level of CXCL1. In some embodiments, the methods provided herein comprise measuring the DNA level of IL4. In some embodiments, the methods provided herein comprise measuring the DNA level of IL6. In some embodiments, the methods provided herein comprise measuring the DNA level of IL8. In some embodiments, the methods provided herein comprise measuring the DNA level of MDC. In some embodiments, the methods provided herein comprise measuring the DNA level of IP10. In some embodiments, the methods provided herein comprise measuring the DNA level of GMCSF. In some embodiments, the methods provided herein comprise measuring the DNA level of MIP1a. In some embodiments, the methods provided herein comprise measuring the DNA level of TGFa.

### 5.5. Kits

In one aspect, provided herein is a kit for performing the methods provided herein. In some embodiments, the kit is for determining a pharmacodynamics or pharmacokinetic effect of an inhibitor of IL1α. In other embodiments, the kit is for identifying a subject who is likely to be responsive to a treatment comprising an inhibitor of IL1α. In other embodiments, the kit is for predicting the responsiveness of a subject to a treatment comprising an inhibitor of IL1α. In yet other embodiments, the kit is for monitoring the response of a subject to a treatment comprising an inhibitor of IL1α.

In some embodiments, the kit provided herein comprises one or more agents for measuring levels of one or more biomarkers in a sample selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3, or from a group consisting of GCSF, CXCL1, IL4, IL6, IL8, MDC, IP10, GMCSF, MIP1a and TGFa.

In some embodiments, the kit comprises one or more agents for measuring the levels of one or more biomarkers in a sample, wherein the one or more biomarkers are selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the kit comprises one or more agents for measuring the levels of one or more biomarkers in a sample, wherein the one or more biomarkers are selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In some embodiments, the kit comprises an agent for measuring the level of CCL20. In some embodiments, the kit comprises an agent for measuring the level of CCL22. In some embodiments, the kit comprises an agent for measuring the level of CSF3. In some embodiments, the kit comprises an agent for measuring the level of CXCL1. In some embodiments, the kit comprises an agent for measuring the level of CXCL2. In some embodiments, the kit comprises an agent for measuring the level of CXCL3. In some embodiments, the kit comprises an agent for measuring the level of CXCL5. In some embodiments, the kit comprises an agent for measuring the level of CXCL6. In some embodiments, the kit comprises an agent for measuring the level of IL6. In some embodiments, the kit comprises an agent for measuring the level of IL8. In some embodiments, the kit comprises an agent for measuring the level of PTGS2. In some embodiments, the kit comprises an agent for measuring the level of CCL3. In some embodiments, the kit comprises an agent for measuring the level of CCL4. In some embodiments, the kit comprises an agent for measuring the level of CCL8. In some embodiments, the kit comprises an agent for measuring the level of CFB. In some embodiments, the kit comprises an agent for measuring the level of IL1B. In some embodiments, the kit comprises an agent for measuring the level of MMP3. In some embodiments, the kit comprises an agent for measuring the level of GCSF. In some embodiments, the kit comprises an agent for measuring the level of CXCL1. In some embodiments, the kit comprises an agent for measuring the level of IL4. In some embodiments, the kit comprises an agent for measuring the level of IL6. In some embodiments, the kit comprises an agent for measuring the level of IL8. In some embodiments, the kit comprises an agent for measuring the level of MDC. In some embodiments, the kit comprises an agent for measuring the level of IP10. In some embodiments, the kit comprises an agent for measuring the level of GMCSF. In some embodiments, the kit comprises an agent for measuring the level of MIP1a. In some embodiments, the kit comprises an agent for measuring the level of TGFa.

In one embodiment, the kit comprises an agent for measuring the level of CSF3 (GCSF). In another embodiment, the kit comprises an agent for measuring the level of CXCL1. In another embodiment, the kit comprises an agent for measuring the level of IL6.

In one embodiment, the kit comprises agents for measuring the levels of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the kit comprises agents for measuring the levels of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

In one embodiment, the kit comprises agents for measuring the levels of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

In one embodiment, the kit comprises agents for measuring the levels of CCL20, CCL22, CSF3(GCSF), CXCL1, CXCL2, CXCL3, CXCL5, CXCL6, IL6, IL8, and PTGS2.

In one embodiment, the kit comprises agents for measuring the levels of CCL20, CCL3, CCL4, CCL8, CFB, CSF3(GCSF), CXCL1, CXCL2, CXCL3, CXCL5, IL1B, IL6, IL8, MMP3, and PTGS2.

In one embodiment, the kit comprises agents for measuring the levels of CCL20, CSF3(GCSF), CXCL1, CXCL2, CXCL3, CXCL5, IL6, IL8, and PTGS2.

In one embodiment, the kit comprises agents for measuring the levels of CSF3 (GCSF), CXCL1 and IL6.

In one embodiment, the kit comprises agents for measuring the levels of GCSF, CXCL1, IL4, IL6, IL8, MDC, IP10, GMCSF, MIP1a and TGFa.

In one embodiment, the kit comprises agents for measuring the levels of GCSF, CXCL1, IL4, IL6, IL8, MDC and IP10.

In one embodiment, the kit comprises agents for measuring the levels of CXCL1, GCSF, GMCSF, IL6, IL8, MIP1a and TGFa.

In one embodiment, the kit comprises agents for measuring the levels of GCSF, CXCL1, IL6 and IL8.

In some embodiments, the agent is for measuring the protein level of the above biomarker. In other embodiments, the agent is for measuring the mRNA level of the above biomarker. In other embodiments, the agent is for measuring the cDNA level of the above biomarker. In yet other embodiments, the agent is for measuring the DNA level of the above biomarker.

In some embodiments, the kit provided herein further comprises a control agent so that the effect of the tested inhibitor of IL1α can be compared to that of the control agent. In some embodiments, the control agent is a positive control agent that inhibits IL1α. In some embodiments, the positive control agent is an anti-IL1α antibody. In other embodiments, the control agent is a negative control agent that does not inhibit IL1α.

In some embodiments, the kit provided herein further comprises a tool suitable for obtaining a sample from a subject, for example, a tool suitable for obtaining a skin biopsy sample.

In some embodiments, the kit provided herein further comprises culture medium for culturing a sample obtained from a subject. In some embodiments, the culture medium is suitable for culturing a skin biopsy sample.

The kit provided herein can further comprises a tool or a device for administering an agent to a sample. Examples of such devices include, but are not limited to, syringes, drip bags, patches, and inhalers. Kits may further comprise pharmaceutically acceptable vehicles that can be used to administer one or more ingredients. For example, if an ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to, water for injection USP; aqueous vehicles (such as, but not limited to, sodium chloride injection, Ringer's injection, dextrose injection, dextrose and sodium chloride injection, and lactated Ringer's injection); water-miscible vehicles (such as, but not limited to, ethyl alcohol, polyethylene glycol, and polypropylene glycol); and non-aqueous vehicles (such as, but not limited to, corn oil, cottonseed oil, peanut oil, sesame oil, ethyl oleate, isopropyl myristate, and benzyl benzoate).

The kit may also contain an instruction on how to use the kit and apply various components of the kit. The kit may also include instructions on how to interpret the measurement, e.g., by providing a reference level of the gene.

In certain embodiments, provided herein is a kit for detecting the mRNA level of one or more genes. In certain embodiments, the kit comprises one or more probes that bind specifically to the mRNAs of the one or more genes. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more genes. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an organ, an organelle, a biological fluid, a blood sample, a urine sample, or a skin sample.

In another embodiment, the kit comprises a solid support, nucleic acids attached to the support, where the nucleic acids are complementary to at least 20, 50, 100, 200, 350, or more bases of mRNA, and a means for detecting the expression of the mRNA in a biological sample.

In a specific embodiment, the kit comprises components for isolating RNA. In another specific embodiment, the kit comprises components for conducting RT-PCR, qRT-PCR, deep sequencing, or microarray.

In certain embodiments, the kits provided herein employ means for detecting the expression of a biomarker by qRT-PCR, microarray, flow cytometry, or immunofluorescence. In other embodiments, the expression of the biomarker is measured by ELISA-based methodologies or other similar methods known in the art.

In another specific embodiment, the kit comprises components for isolating protein. In another specific embodiment, the kit comprises components for conducting flow cytometry or ELISA.

In another aspect, provided herein are kits for determining level of a gene that supply the materials necessary to measure the abundance of one or more gene products (e.g., 1, 2, 3, 4, 5, 6, 7, 8, 9, 10, 11, 12, 13, 14, 15, 16, or more genes) provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more gene products provided herein, or any combination thereof. In some embodiments, such kits may include primers for PCR of either the RNA product or the cDNA copy of the RNA product of the genes. In some embodiments, such kits may include primers for PCR as well as probes for qPCR. In some embodiments, such kits may include multiple primers and multiple probes, wherein some of the probes have different fluorophores so as to permit simultaneously measuring multiple gene products provided herein. In some embodiments, such kits may further include materials and reagents for creating cDNA from RNA. In some embodiments, such kits may include antibodies specific for the protein products of the gene provided herein. Such kits may additionally comprise materials and reagents for isolating RNA and/or proteins from a biological sample. In addition, such kits may include materials and reagents for synthesizing cDNA from RNA isolated from a biological sample. In some embodiments, such kits may include a computer program product embedded on computer readable media for predicting whether a patient is clinically sensitive to a compound.

In some embodiments, the kits may include a computer program product embedded on a computer readable media along with instructions.

In some embodiments, such kits measure the expression of one or more nucleic acid products of the genes provided herein. In accordance with this embodiment, the kits may comprise materials and reagents that are necessary for measuring the expression of particular nucleic acid products of the genes provided herein. For example, a microarray or RT-PCR kit may be produced for a specific condition and contain only those reagents and materials necessary for measuring the levels of specific RNA transcript products of the genes provided herein, to predict whether a patient is clinically sensitive to a compound. Alternatively, in some embodiments, the kits can comprise materials and reagents necessary for measuring the expression of particular nucleic acid products of genes other than the genes provided herein. For example, in certain embodiments, the kits comprise materials and reagents necessary for measuring the expression levels of 1, 2, 3, 4, 5, 6, 7, 8, 9, 10, 11, 12, 13, 14, 15, 16, 17, 20, 25, 30, 35, 40, 45, 50, or more of the genes, in addition to reagents and materials necessary for measuring the expression levels of the genes provided herein. In other embodiments, the kits contain reagents and materials necessary for measuring the expression levels of at least 1, at least 2, at least 3, at least 4, at least 5, at least 6, at least 7, at least 8, at least 9, at least 10, or more of the genes provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not the genes provided herein.

For nucleic acid microarray kits, the kits generally comprise probes attached to a solid support surface. In one such embodiment, probes can be either oligonucleotides or longer probes including probes ranging from 150 nucleotides to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from performing the assay. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own suitable container.

In certain embodiments, a nucleic acid microarray kit comprises materials and reagents necessary for measuring the expression levels of 1, 2, 3, 4, 5, 6, 7, 8, 9, 10, 15, or more of the genes provided herein, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those of the genes provided herein. In other embodiments, a nucleic acid microarray kit contains reagents and materials necessary for measuring the expression levels of at least 1, at least 2, at least 3, at least 4, at least 5, at least 6, at least 7, at least 8, at least 9, at least 10, at least 15, or more of the genes provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the genes provided herein. In another embodiment, a nucleic acid microarray kit contains reagents and materials necessary for measuring the expression levels of at least 1, at least 2, at least 3, at least 4, at least 5, at least 6, at least 7, at least 8, at least 9, at least 10, at least 15, or more of the genes of the genes provided herein, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000, or 500-1000 genes that are not the genes provided herein.

For quantitative PCR, the kits generally comprise pre-selected primers specific for particular nucleic acid sequences. The quantitative PCR kits may also comprise enzymes suitable for amplifying nucleic acids (e.g., polymerases such as Taq polymerase), deoxynucleotides, and buffers needed for amplification reaction. The quantitative PCR kits may also comprise probes specific for the nucleic acid sequences associated with or indicative of a condition. The probes may or may not be labeled with a fluorophore. The probes may or may not be labeled with a quencher molecule. In some embodiments, the quantitative PCR kits also comprise components suitable for reverse-transcribing RNA, including enzymes (e.g., reverse transcriptases such as AMV, MMLV, and the like) and primers for reverse transcription along with deoxynucleotides and buffers needed for reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the reaction and methods for interpreting and analyzing the data resulting from performing the reaction.

For antibody-based kits, the kit can comprise, for example: (1) a first antibody (which may or may not be attached to a solid support) that binds to a peptide, polypeptide or protein of interest; and, optionally, (2) a second, different antibody that binds to either the first antibody or the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulting from performing the assay.

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support, and the like, are also contemplated in relation to any of the various methods and/or kits provided herein.

Certain embodiments of the invention are illustrated by the following non-limiting examples.

### 6. EXAMPLES

The examples below are carried out using standard techniques, which are well known and routine to those of skill in the art, except where otherwise described in detail. The examples are intended to be merely illustrative.

### 6.1. Example 1—Expression of IL1α can be Provoked in Skin Injury Ex Vivo Biopsy Explant Culture Model

This example illustrates an assay provided herein that enables measurements of pharmacodynamics and/or pharmacokinetic effects of an IL1α blocker (or an IL1α inhibitor).

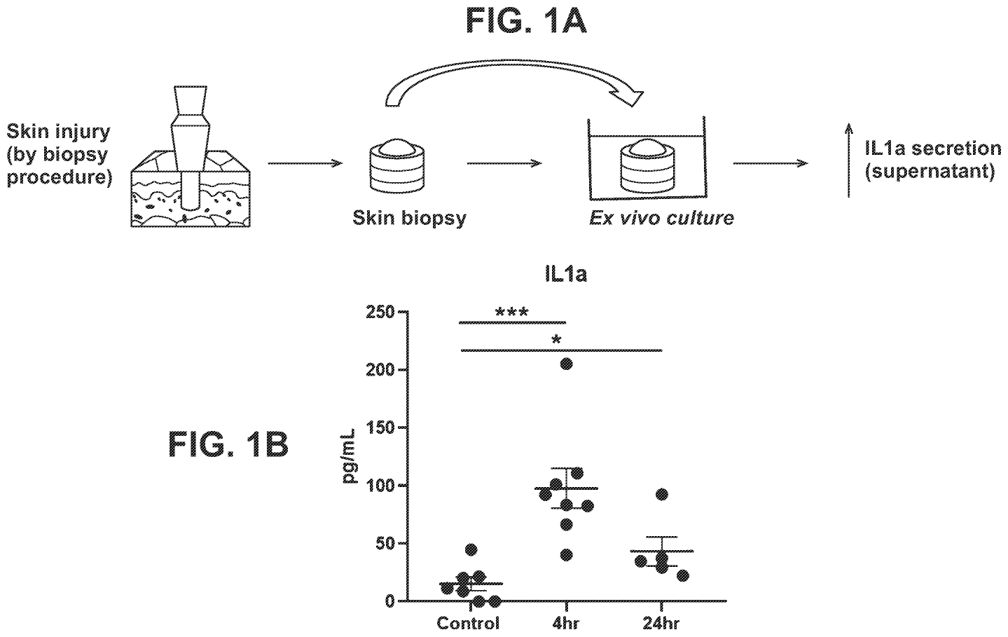

Inflammation is an important response of the immune system to cellular stress, injury or infection with the purpose of restoring tissue homeostasis. IL1α, a member of the IL1 family of cytokines, is a potent inflammatory cytokine that is released early during the inflammatory response and function to further activate the process. IL1α has also been recently shown to mediate the skin injury induced inflammatory response (SID 2019 Abstract/Poster, M J Turner, Indiana U). In this example, expression of IL1α was shown elevated in ex vivo skin biopsy explant-based assay. In summary, skin biopsies were obtained from healthy piece of skin and were cultured ex vivo (see FIG. 1A). As shown in FIG. 1B, level of IL1α was elevated in culture supernatant of skin biopsy explant cultured ex vivo for 4 hours or 24 hours following skin injury (induced by biopsy procedure).

### 6.2. Example 2—Ex Vivo Biopsy Explant Culture Model can be Utilized to Evaluate Effects of IL1α Blockade on Skin Inflammatory Responses

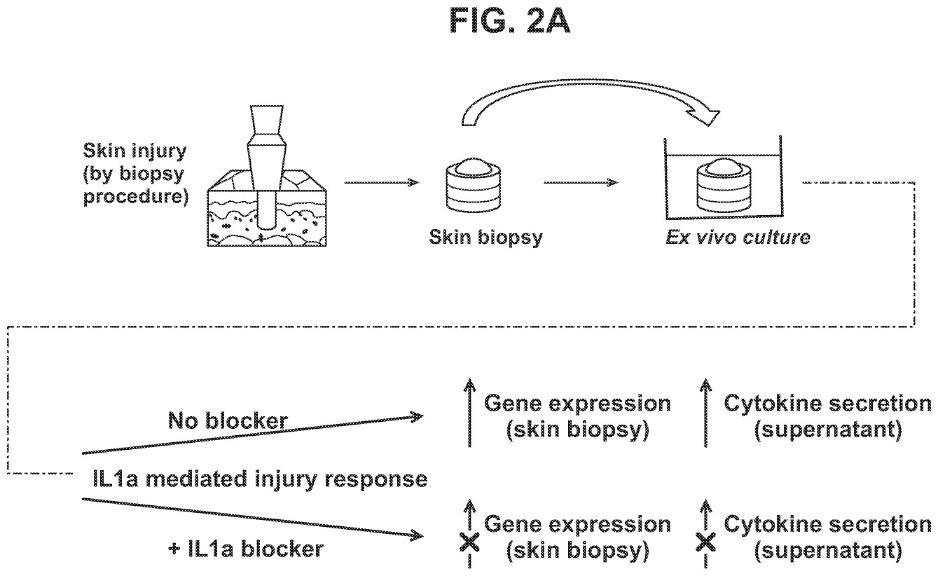

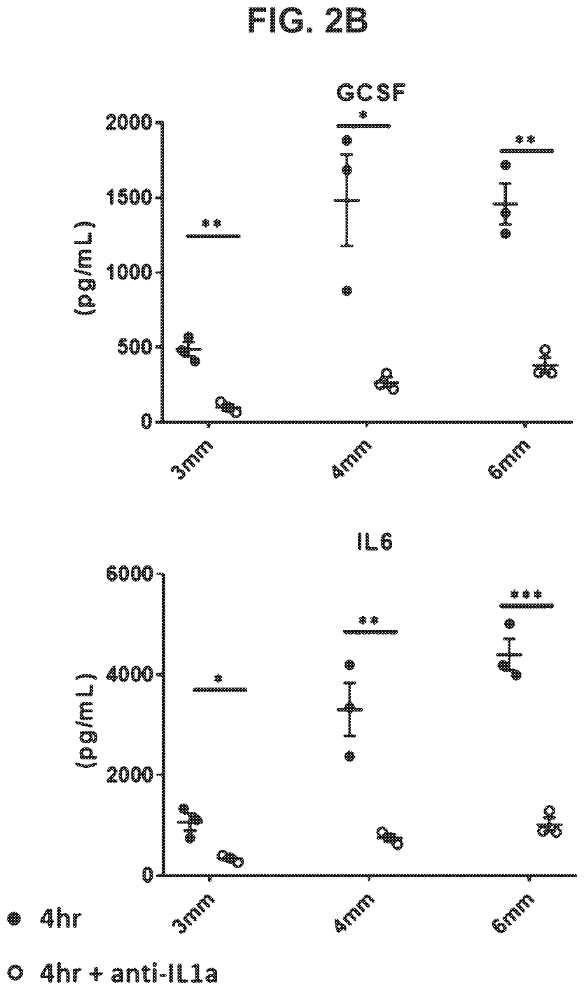

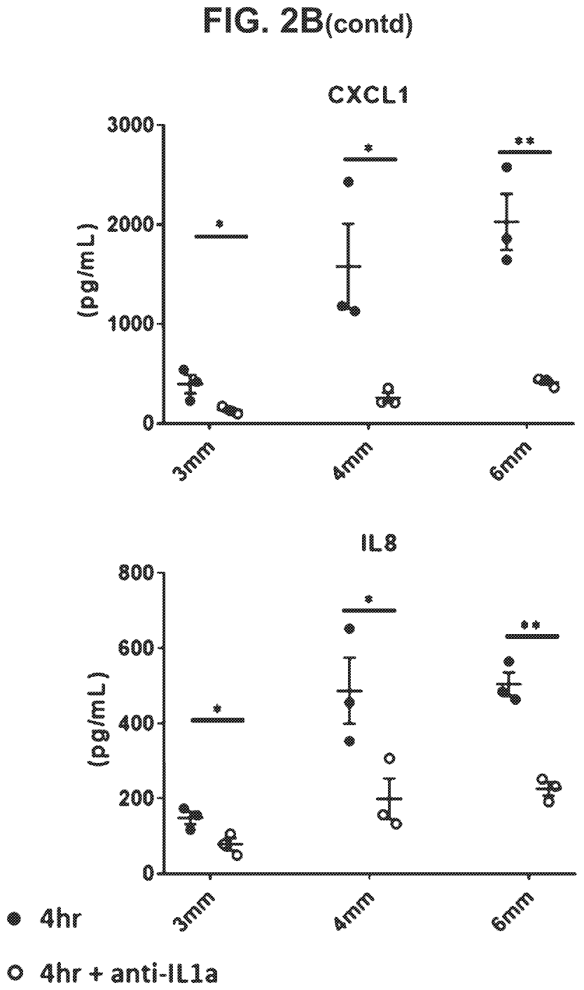

Next, we assessed the feasibility of using the ex vivo biopsy explant culture model to measure the impact of IL1α blockade of skin inflammatory responses. Briefly, skin biopsies were obtained from healthy piece of skin and were cultured ex vivo with or without anti-IL1α antibody (R&D, clone 4414). The readouts to monitor inflammatory response were gene expression level in skin biopsy and cytokine level in the culture supernatant. The effects of using different biopsy size to set up the ex vivo biopsy explant culture model were evaluated. While larger skin biopsy is preferred from experimental purpose (increased cytokine secretion level with larger skin biopsy), small skin biopsy is preferred for clinical implementation. To assess the impact of skin biopsy size on the ability to measure effects of IL1α blockade, 3 mm, 4 mm and 6 mm skin biopsy were compared. Luminex analysis of the culture supernatant showed that the level of GCSF, CXCL1, IL6 and IL8 were very low in control (data not shown), were elevated following 4 hours of culture and were significantly reduced by treatment with anti-IL1α antibody (see FIG. 2B). While treatment with anti-IL1α antibody significantly reduced cytokine level in all the biopsy size evaluated, the dynamic range (difference between 4 hr and 4 hr+anti-IL1α) was much smaller in 3 mm biopsy size. On the other hand, the range was larger and was similar between 4 mm and 6 mm biopsy size. Thus, 4 mm biopsy size was used in the subsequent development of the assay.

### 6.3. Example 3—Lidocaine Injection Affects the Ability of Anti-IL1α to Reduce Level of GCSF, CXCL1 and IL8 in Culture Supernatant at 4-Hour, but not at 24-Hour Time Point

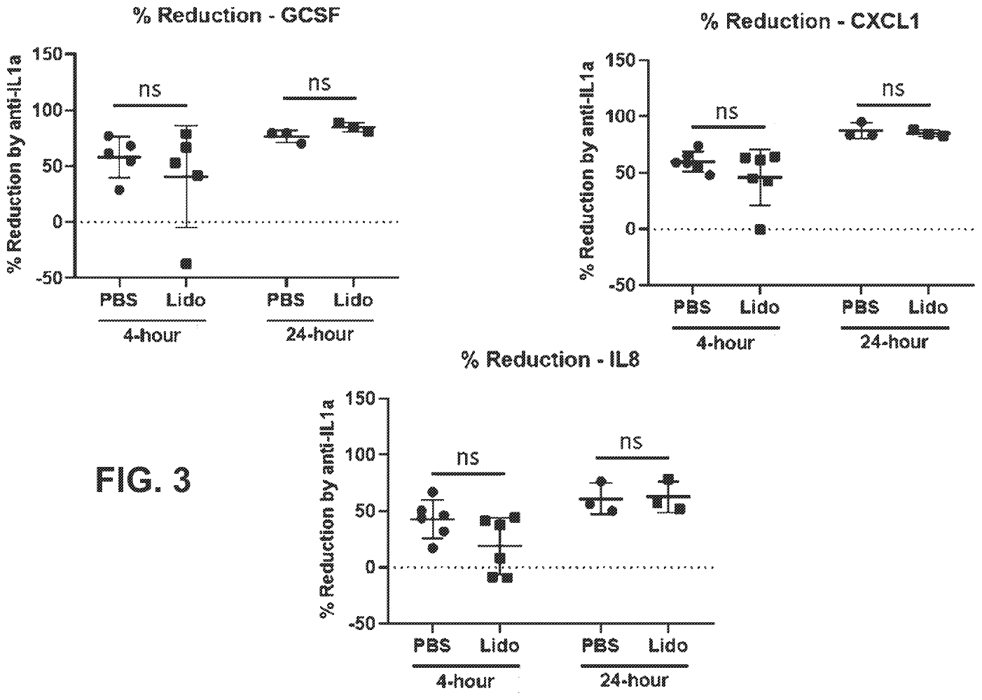

In the clinic, local anesthetic (injection of 1% lidocaine) is usually used to numb the skin prior to the biopsy procedure. To evaluate the effects of lidocaine injection on the ex vivo biopsy explant culture model, experiments were set up utilizing skin that has been injected with 1 mL of 1% lidocaine or with 1 mL of PBS (control). As shown in FIG. 3, the result suggested that lidocaine can potentially impact the ability of anti-IL1α to reduce level of cytokines in culture supernatant, including GCSF, CXCL1 and IL8 at 4-hour time point; % reduction by anti-IL1α in the lidocaine group showed a trend of being lower compared to the PBS group (though not statistically significant). However, similar % reduction by anti-IL1α treatment was observed in both groups at 24-hour time point. Thus, in some embodiments, the ex vivo biopsy explant culture model provided herein utilizes 24-hour time point.

### 6.4. Example 4—Anti IL1α Treatment Reduces Elevated Level of Certain Cytokines in Supernatant of Ex Vivo Biopsy Explant Culture Model at 24-Hour Time Point

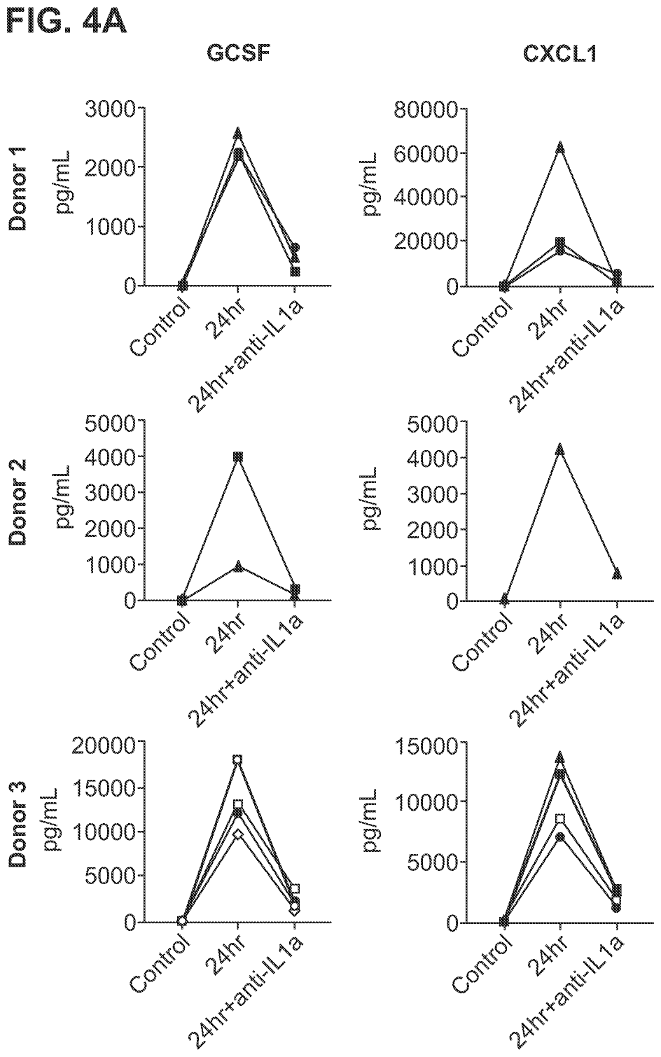

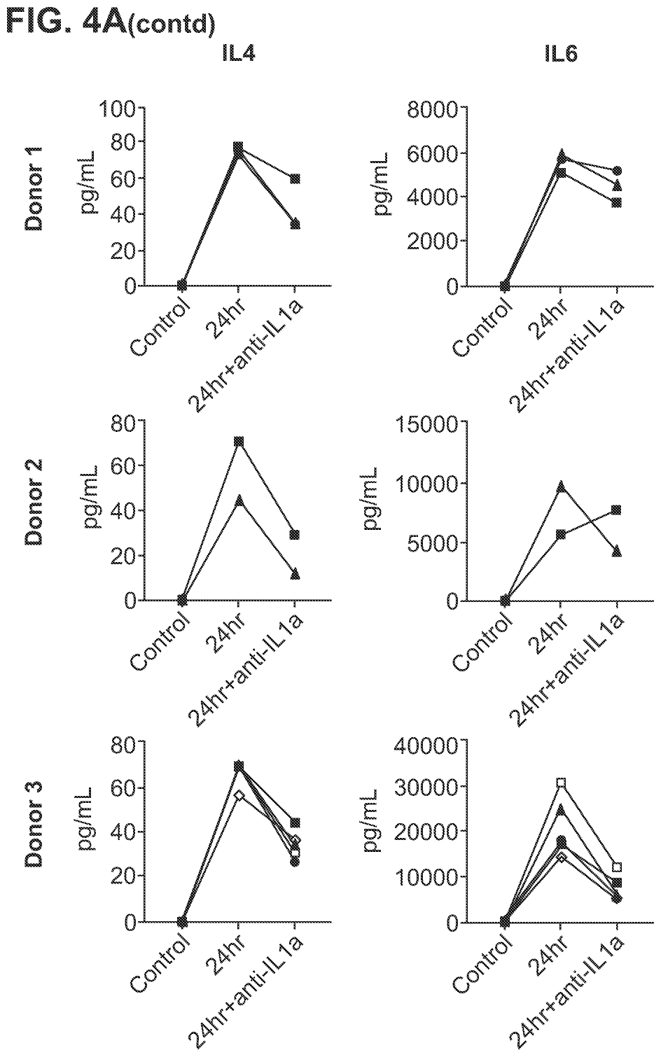

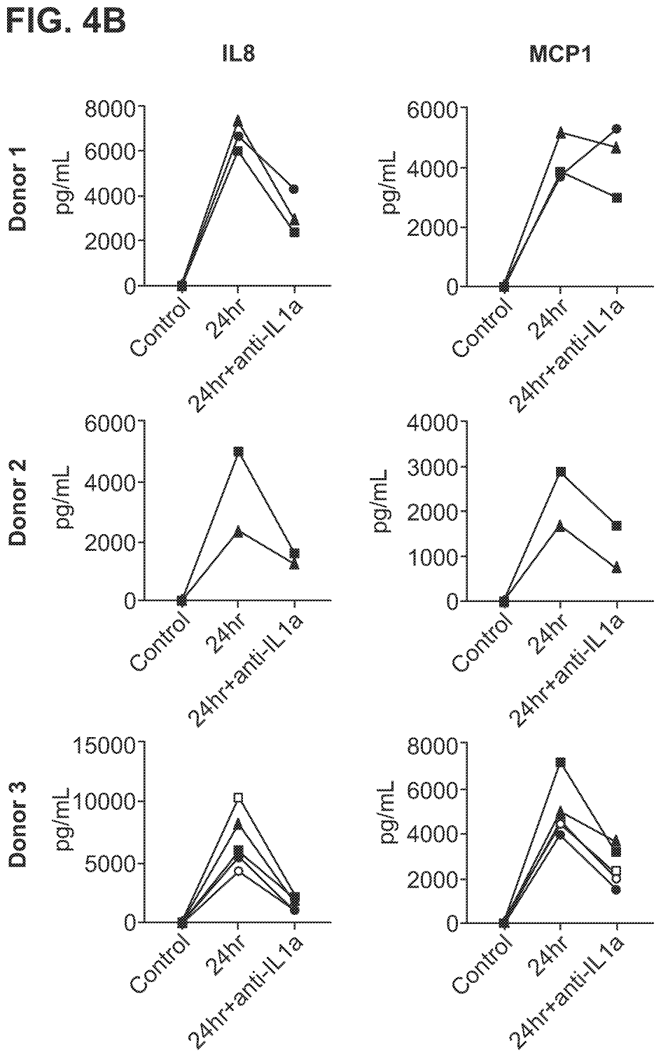

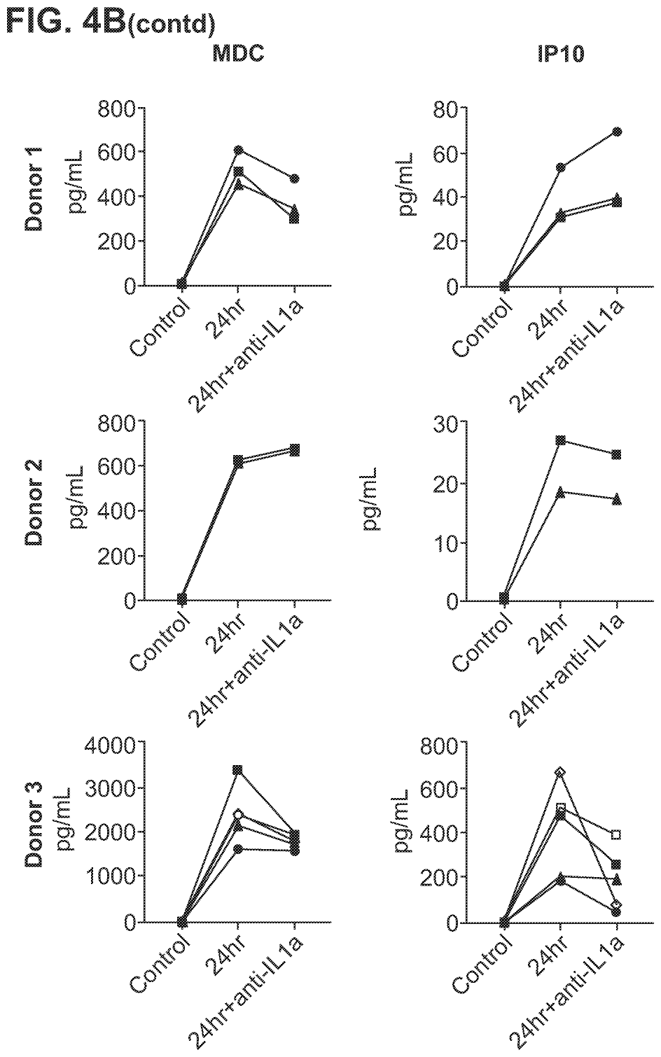

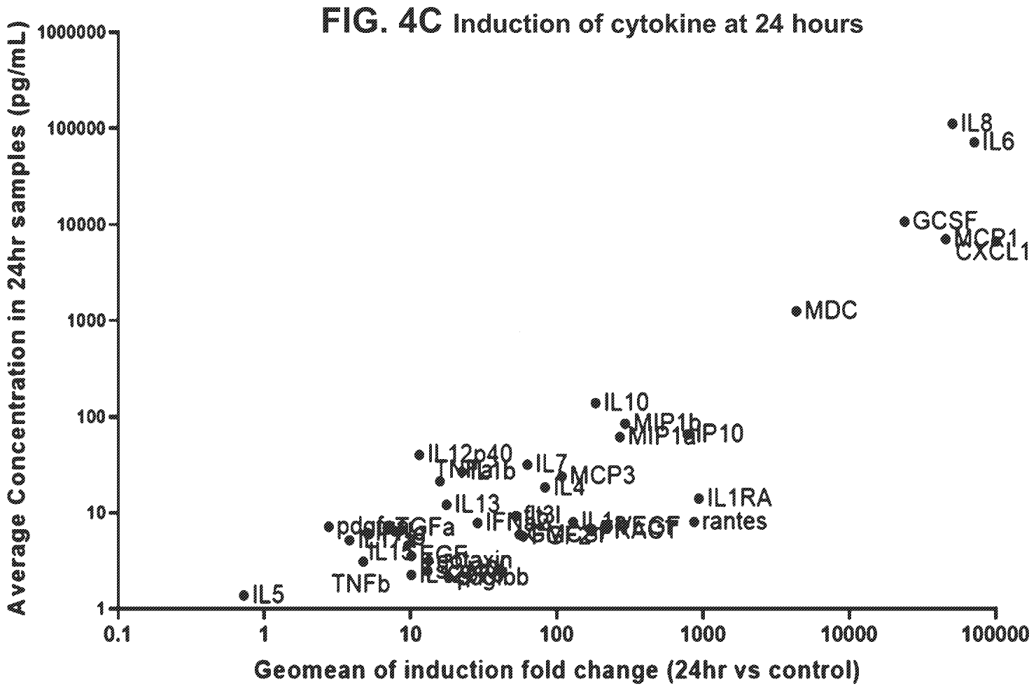

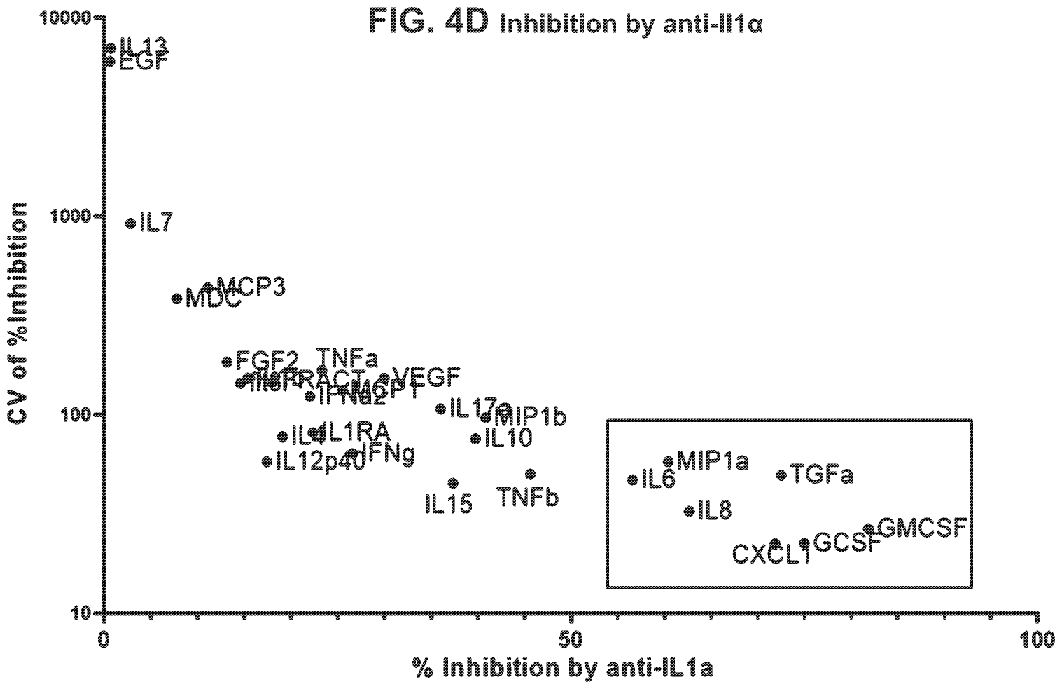

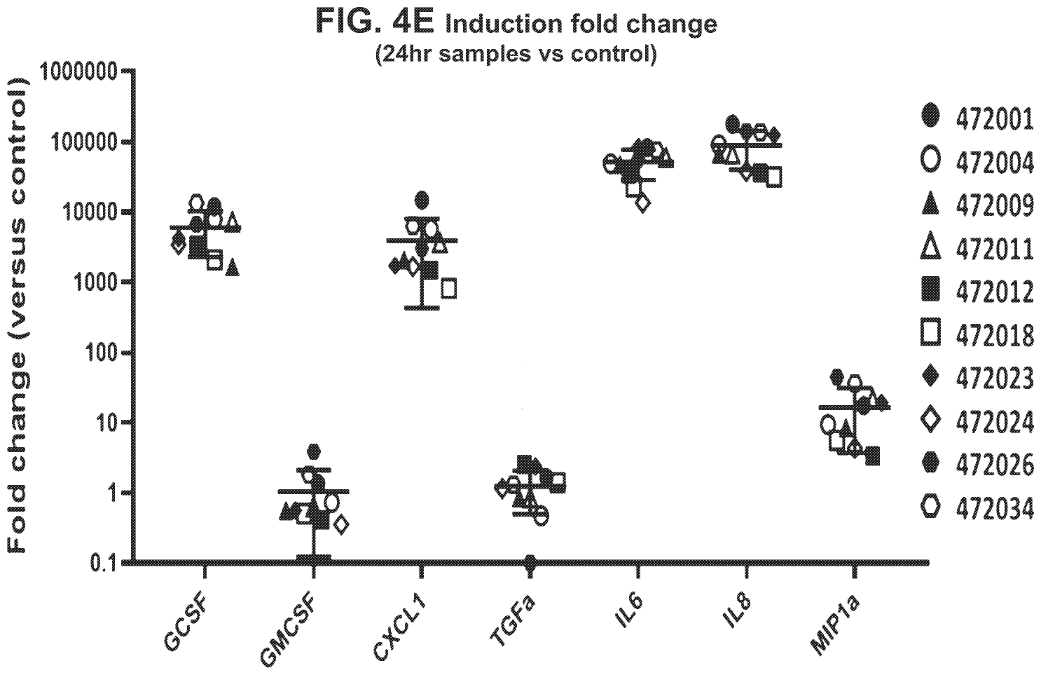

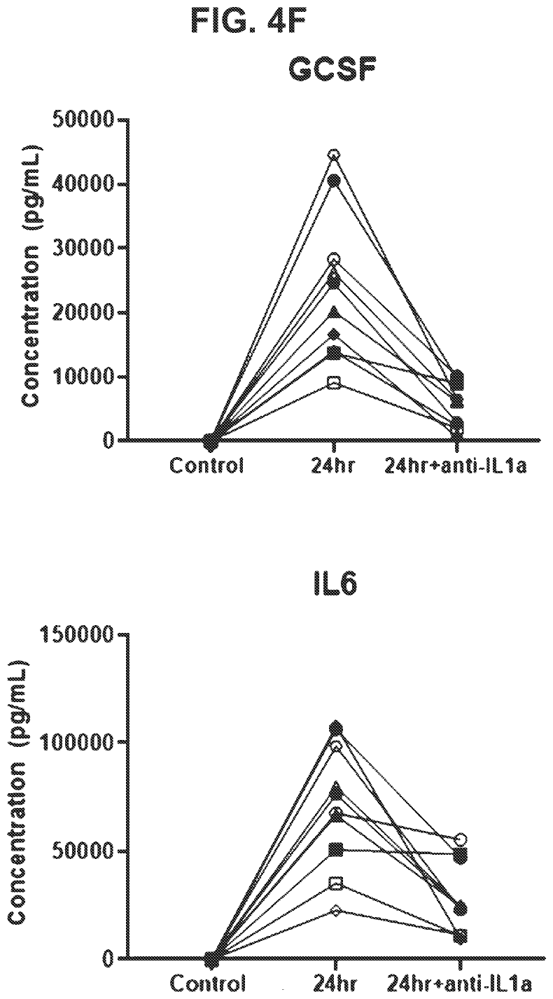

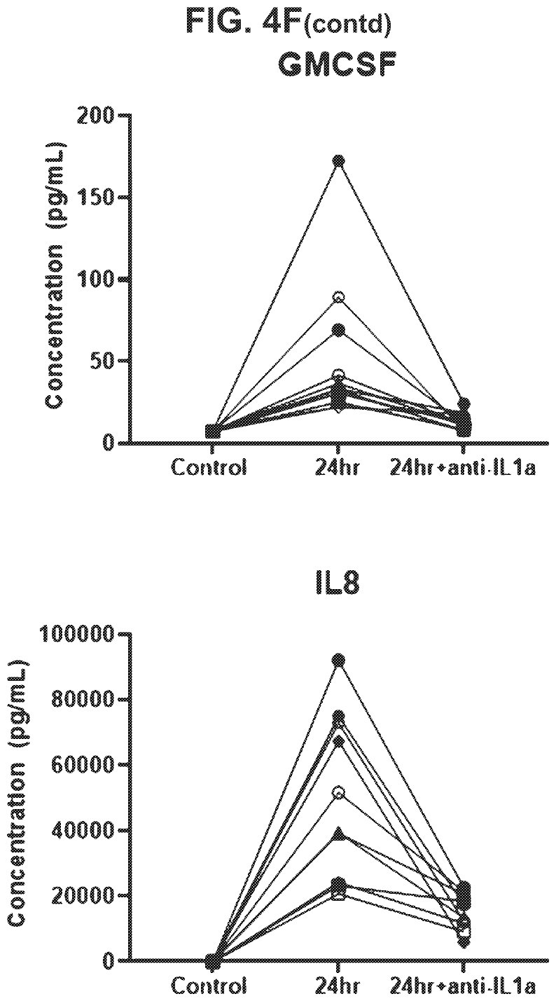

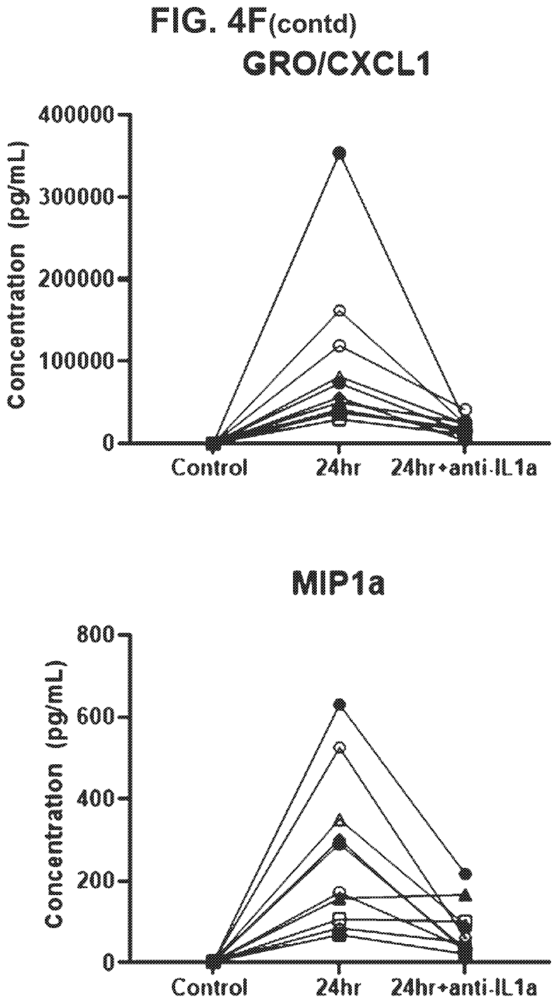

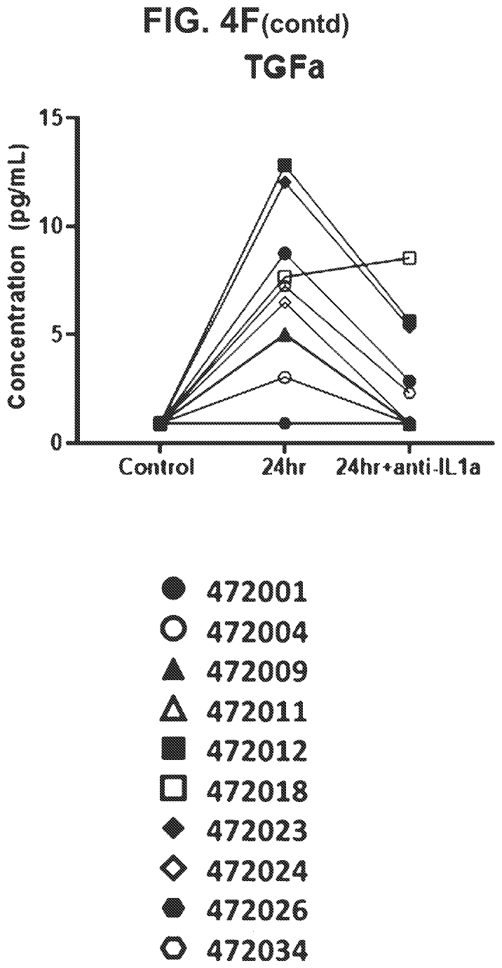

Analysis of the culture supernatant collected from ex vivo biopsy explant culture model (with lidocaine injection) at 24-hour time point identified GCSF, CXCL1, IL4, IL6, IL8, MDC and IP10 as potential biomarkers for measuring effects (e.g., pharmacodynamics effect) of anti-IL1α treatment in this model. The levels of these cytokines were elevated following 24-hour culture and showed a trend of being reduced by anti-IL1α treatment (see FIGS. 4A and 4B).

The ex vivo biopsy explant culture model was further evaluated in 10 healthy volunteers. In brief, 3 skin biopsies (4 mm) were collected from each healthy volunteer under sterile conditions after a local anesthetic agent (lidocaine) injection. One of the biopsies was used as control while the other two biopsies were cultured ex vivo for 24 hours and used to assess effects of anti-IL1α treatment (one of the two biopsies were cultured with anti-IL1α; R&D, clone 4414, 10 ug/mL). Culture supernatant were collected for cytokines measurement while the skin biopsies were used for gene expression analysis. Luminex analysis of the culture supernatant showed the induction of various cytokines following 24 hour of culture (FIG. 4C) and that treatment with anti-IL1α can reduce the level of a subset of cytokines (FIG. 4D). CXCL1, GCSF, GMCSF, IL6, IL8, MIP1a and TGFa were identified as potential biomarkers to measure PD effect of anti IL1α treatment. The level of these cytokines were reduced by anti-IL1α treatment (>50% inhibition) and display low variance (<100%) in their inhibition response. (FIG. 4D). The induction fold change (FIG. 4E) and concentration (FIG. 4F) of these cytokines in the 10 healthy volunteers were plotted.

### 6.5. Example 5—Anti IL1α Treatment Reduces Elevated Expression Levels of Certain Genes in Ex Vivo Biopsy Explant Culture Model (with Lidocaine Injection) at 24-Hour Time Point

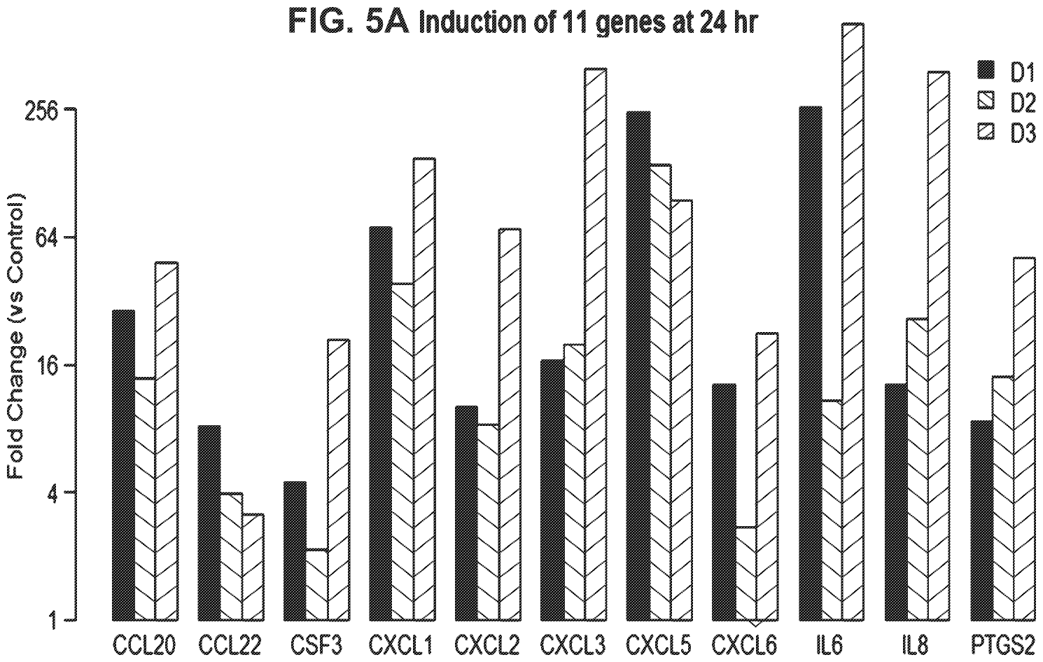

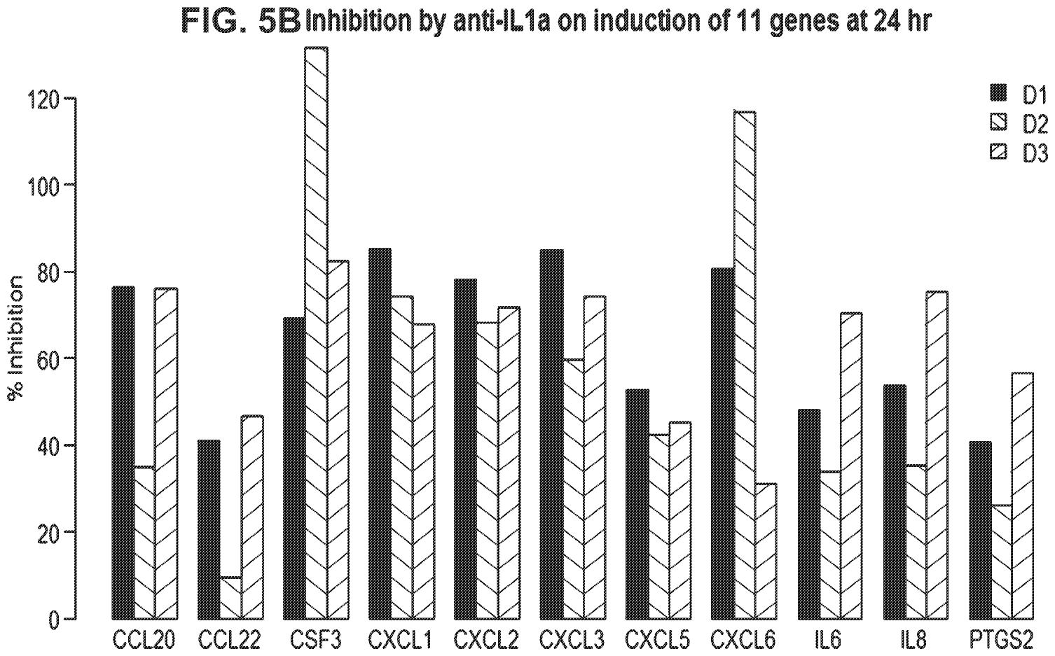

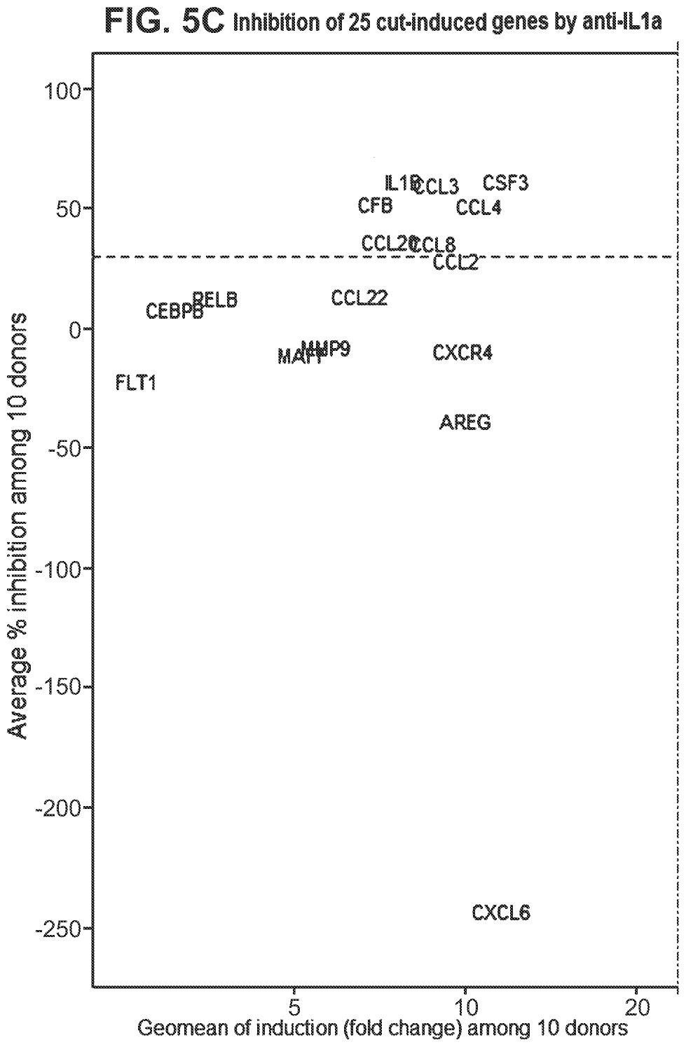

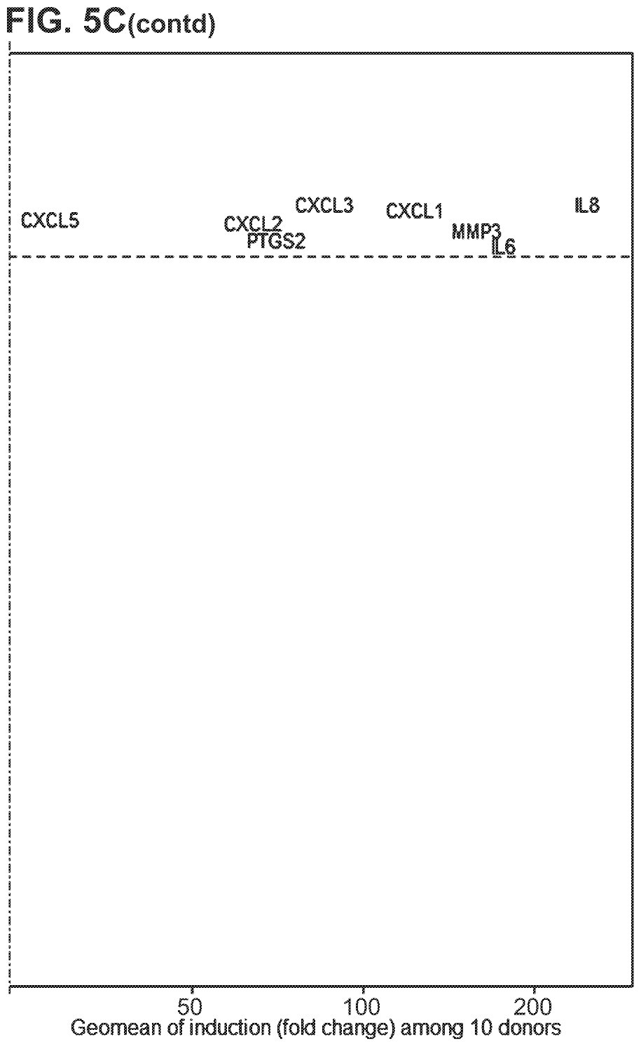

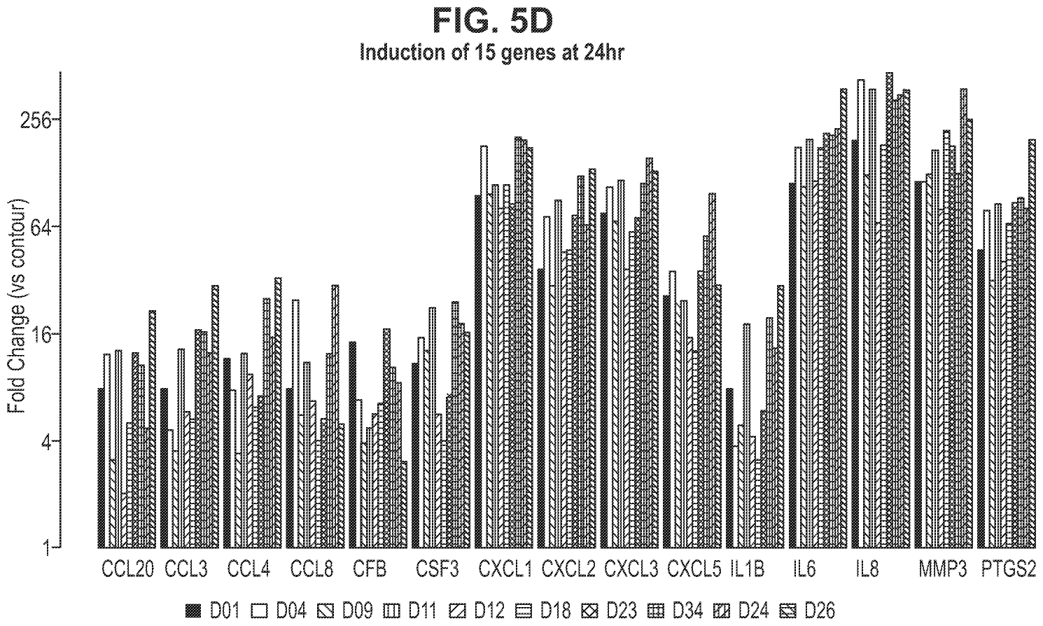

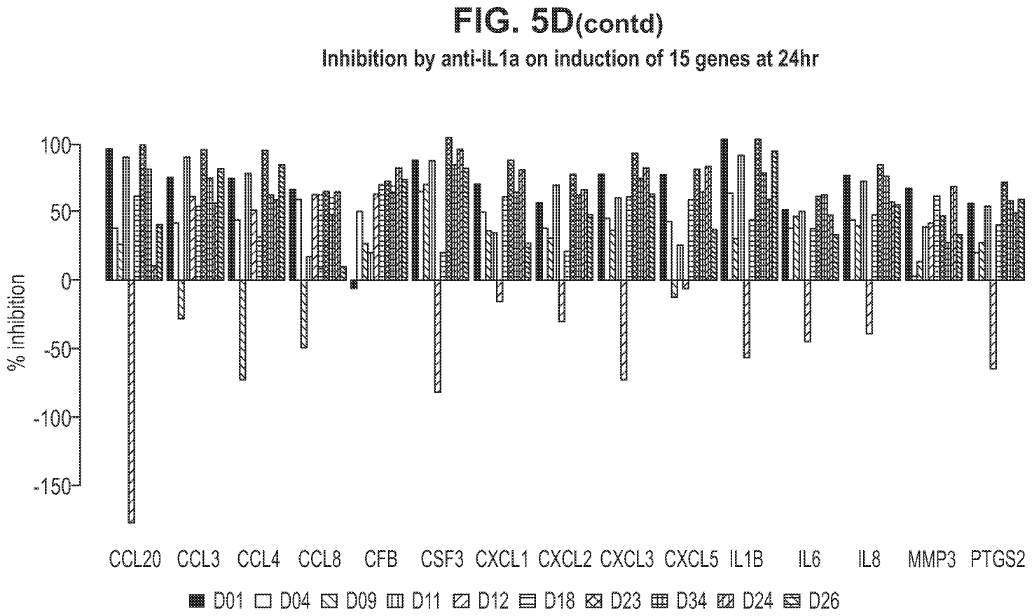

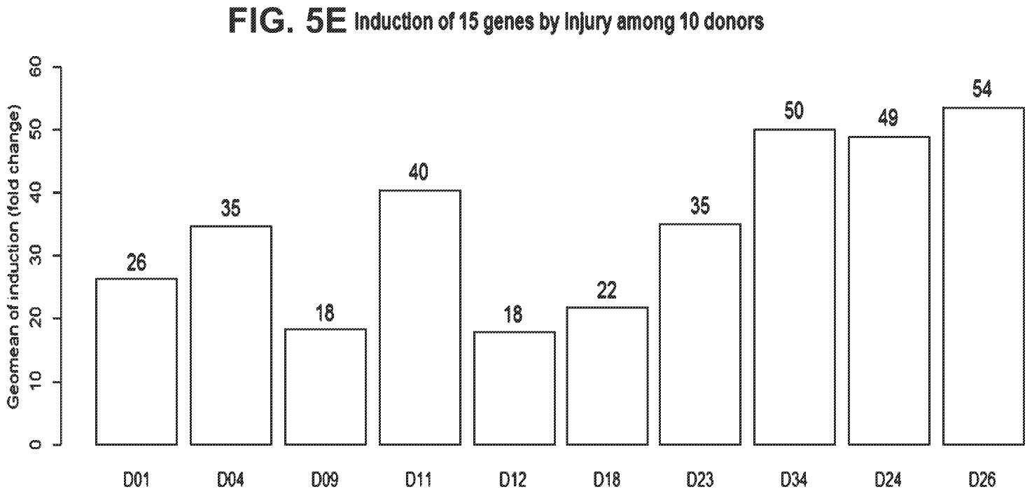

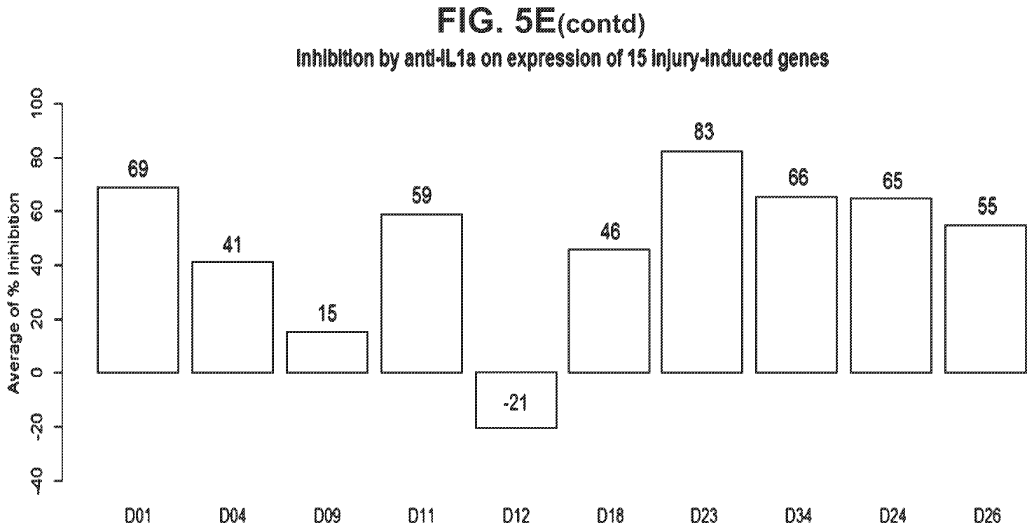

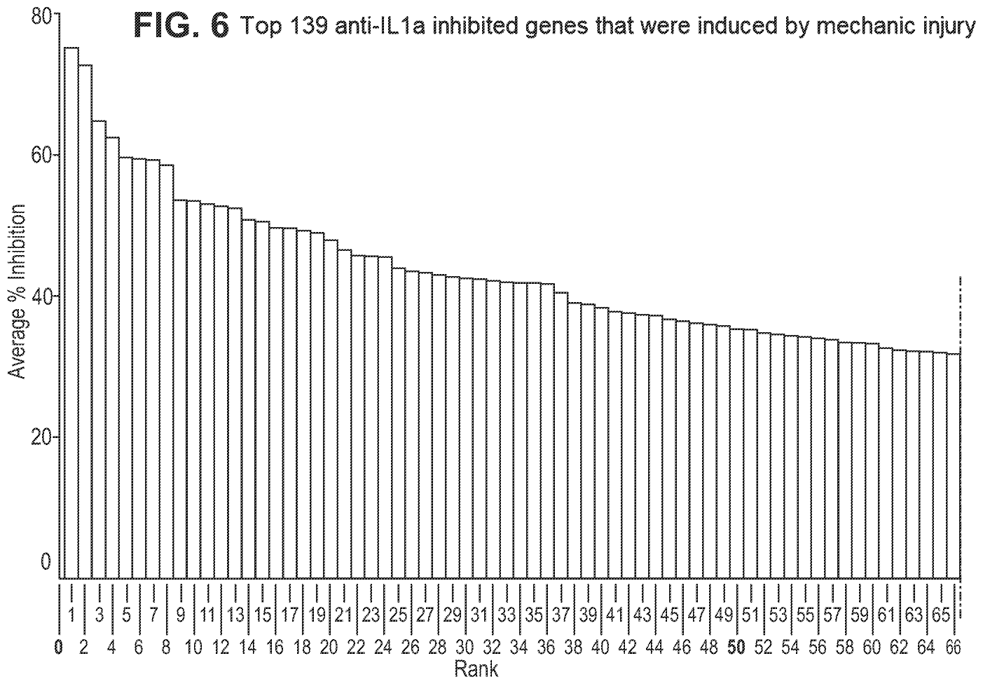

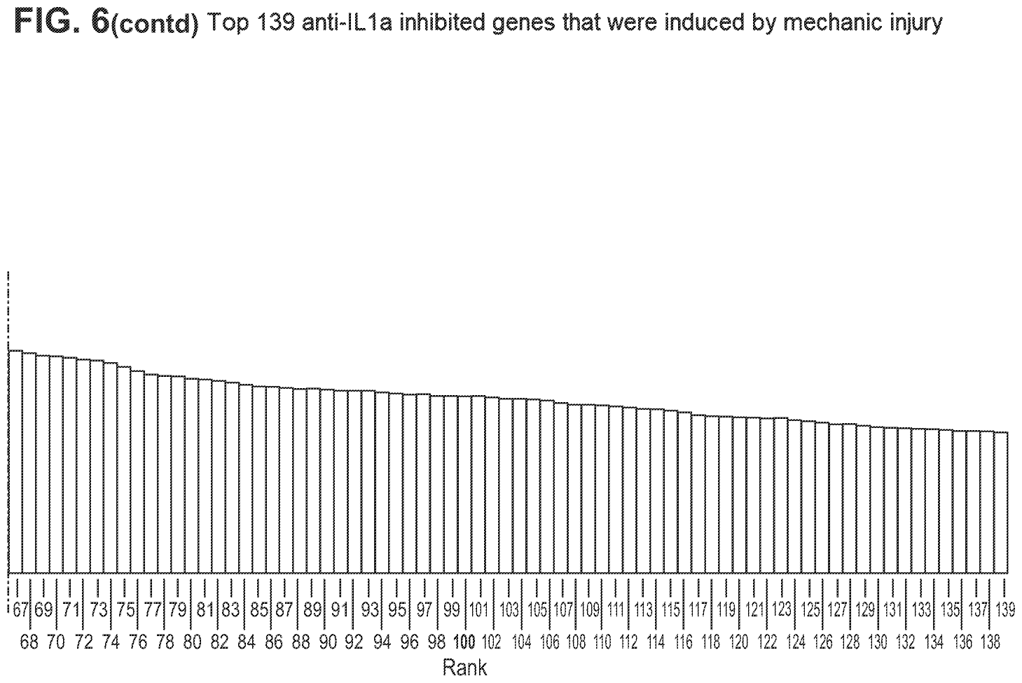

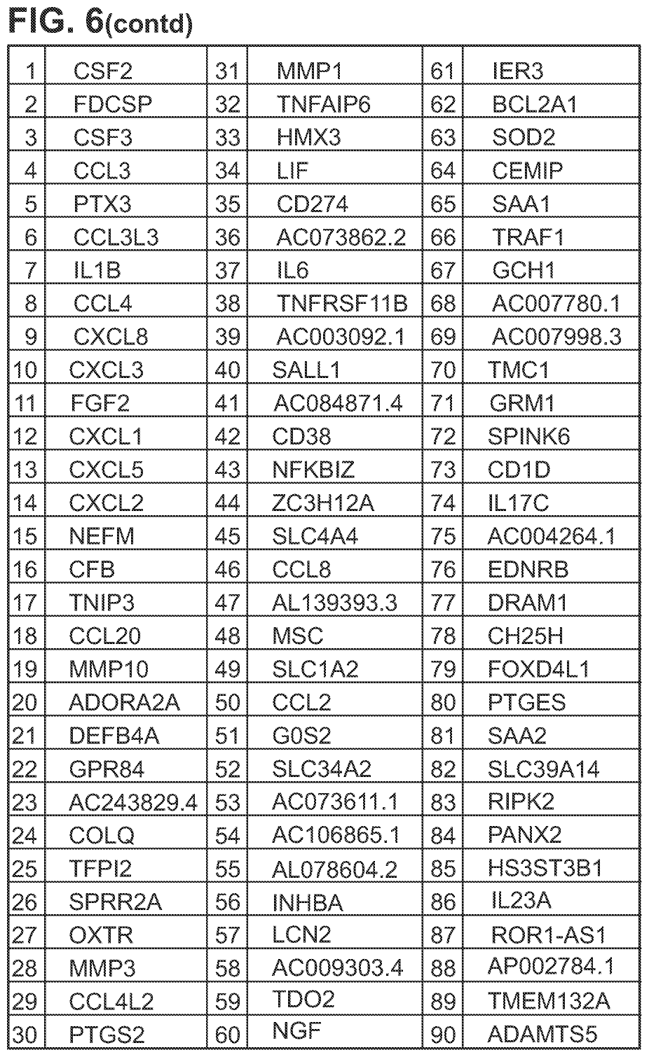

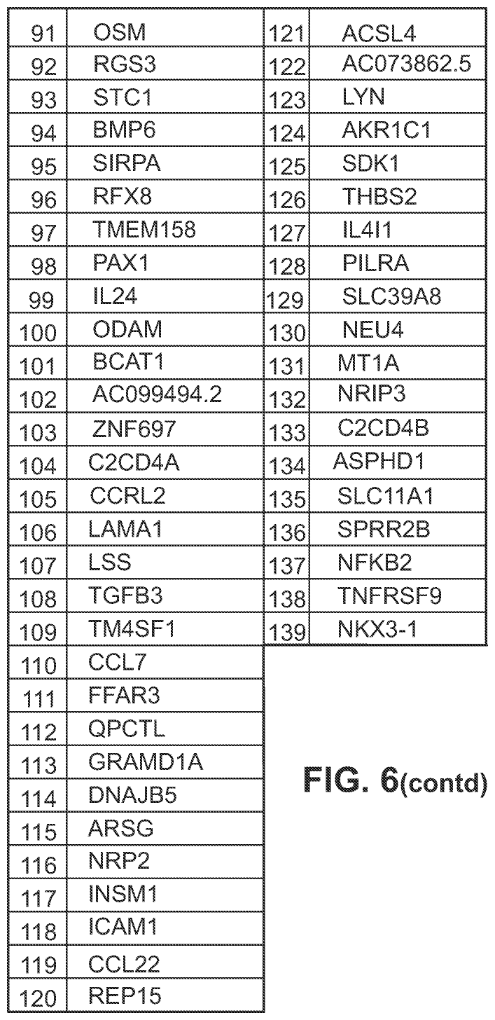

Gene expression of 249 genes in ex vivo biopsy explant culture model (with lidocaine injection) were measured using Nanostring platform. 40 of the 249 tested genes were induced at 24-hour time point, with geometric mean of fold change>2 among skin samples derived from three donors. Surprisingly, elevated gene expression in 11 of the 40 induced genes was inhibited by anti-IL1α treatment, with an average of inhibition>30% (see FIGS. 5A and 5B); these 11 genes were defined as IL1α signature for measuring effects (e.g., pharmacodynamics) of anti-IL1α treatment, e.g., in this model.

The 11 genes identified are listed in the table below.

| Gene | Accession # | SEQ ID NO: |
|---|---|---|
| CCL20 | NM_004591.1 | 1 |
| CCL22 | NM_002990.3 | 2 |
| CSF3 | NM_000759.2 | 3 |
| CXCL1 | NM_001511.1 | 4 |
| CXCL2 | NM_002089.3 | 5 |
| CXCL3 | NM_002090.2 | 6 |
| CXCL5 | NM_002994.3 | 7 |
| CXCL6 | NM_002993.3 | 8 |
| IL6 | NM_000600.1 | 9 |
| IL8 | NM_000584.2 | 10 |
| PTGS2 | NM_000963.1 | 11 |

In a further study, RNA expression of 249 genes, in addition to 6 housekeeping genes, 8 negative genes, and 6 positive genes, were measured using Nanostring in 30 skin biopsies from ten donors, with three skin biopsies per donor. For each donor, one biopsy was assayed right after collection (control), the second biopsy was cultured for 24 hour (24 hr), and the third biopsy was culture for 24 hour with the presence of anti-IL1α antibody (24 hr+anti-IL1α).

The 30 skin biopsies from ten donors were assayed in three runs, with up to 12 samples from four donors assayed in one run. The lower limit of detection (LLOD) across all samples in one run were determined as the maximal value of the reads of the 8 negative genes among all tested samples. Quantification (reads) below LLOD in any one of the 249 genes in each sample were assigned as the LLOD.

After setting the LLOD, quantification (reads) of 249 genes in each sample was further normalized across all 30 samples, by the geometric mean of the reads of all 249 genes to ensure the geometric means are the same across all samples after normalization.

Log 2 transformation was applied on normalized reads, and differential gene expression between two comparing conditions was evaluated using paired t test on log 2 transformed normalized reads, followed by multiple comparison correction using false discovery rate (FDR).

Among the 249 genes evaluated, 25 genes were induced after 24-hour culture (vs control) with fold change>2 in each of the 10 donors. The % inhibition by IL1α antibody on induced expression of the 25 cut-induced genes were further calculated in each donor. 15 of the 25 induced genes had the average of inhibition (among the 10 donors)>30%, and were defined as IL1α signature (see FIG. 5C, FIG. 5D, and FIG. 5E). The 15 genes identified are listed in the table below.

| Gene | Accession # | SEQ ID NO: |
|---|---|---|
| CCL20 | NM_004591.1 | 1 |
| CCL3 | NM_002983.2 | 12 |
| CCL4 | NM_002984.2 | 13 |
| CCL8 | NM_005623.2 | 14 |
| CFB | NM_001710.5 | 15 |
| CSF3 | NM_000759.2 | 3 |
| CXCL1 | NM_001511.1 | 4 |
| CXCL2 | NM_002089.3 | 5 |
| CXCL3 | NM_002090.2 | 6 |
| CXCL5 | NM_002994.3 | 7 |
| IL1B | NM_000576.2 | 17 |
| IL6 | NM_000600.1 | 9 |
| IL8 | NM_000584.2 | 10 |
| MMP3 | NM_002422.3 | 18 |
| PTGS2 | NM_000963.1 | 11 |

The sequences of the above mentioned genes are as follows:

Homo sapiens chemokine (C-C motif) ligand 20 (CCL20), mRNA (NCBI Reference Sequence: NM_004591.1)—SEQ ID NO: 1.

Homo sapiens chemokine (C-C motif) ligand 22 (CCL22), mRNA (NCBI Reference Sequence: NM_002990.3)—SEQ ID NO: 2.

Homo sapiens colony stimulating factor 3 (granulocyte) (CSF3), transcript variant 1, mRNA (NCBI Reference Sequence: NM_000759.2)—SEQ ID NO: 3.

Homo sapiens chemokine (C-X-C motif) ligand 1 (melanoma growth stimulating activity, alpha) (CXCL1), mRNA (NCBI Reference Sequence: NM_001511.1)—SEQ ID NO: 4

Homo sapiens C-X-C motif chemokine ligand 2 (CXCL2), mRNA (NCBI Reference Sequence: NM_002089.3)—SEQ ID NO: 5.

Homo sapiens C-X-C motif chemokine ligand 3 (CXCL3), mRNA (NCBI Reference Sequence: NM_002090.2)—SEQ ID NO: 6.

Homo sapiens chemokine (C-X-C motif) ligand 5 (CXCL5), mRNA (NCBI Reference Sequence: NM_002994.3)—SEQ ID NO: 7.

Homo sapiens C-X-C motif chemokine ligand 6 (CXCL6), mRNA (NCBI Reference Sequence: NM_002993.3)—SEQ ID NO: 8.

Homo sapiens interleukin 6 (interferon, beta 2) (IL6), mRNA (NCBI Reference Sequence: NM_000600.1)—SEQ ID NO: 9.

Homo sapiens interleukin 8 (IL8), mRNA (NCBI Reference Sequence: NM_000584.2)—SEQ ID NO: 10.

Homo sapiens prostaglandin-endoperoxide synthase 2 (prostaglandin G/H synthase and cyclooxygenase) (PTGS2), mRNA (NCBI Reference Sequence: NM_000963.1)—SEQ ID NO: 11.

Homo sapiens C-C motif chemokine ligand 3 (CCL3), mRNA, NCBI Reference Sequence: NM_002983.2—SEQ ID NO: 12.

Homo sapiens chemokine (C-C motif) ligand 4 (CCL4), mRNA, NCBI Reference Sequence: NM_002984.2—SEQ ID NO: 13.

Homo sapiens C-C motif chemokine ligand 8 (CCL8), mRNA, NCBI Reference Sequence: NM_005623.2—SEQ ID NO: 14.

Homo sapiens complement factor B (CFB), mRNA, NCBI Reference Sequence: NM_001710.5—SEQ ID NO: 15.

Homo sapiens C-X-C motif chemokine ligand 2 (CXCL2), mRNA, NCBI Reference Sequence: NM_002089.3—SEQ ID NO: 16.

Homo sapiens interleukin 1 beta (IL1B), mRNA, NCBI Reference Sequence: NM_000576.2—SEQ ID NO: 17.

Homo sapiens matrix metallopeptidase 3 (MMP3), mRNA, NCBI Reference Sequence: NM_002422.3—SEQ ID NO: 18.

In a further study, whole genome transcriptome in the same 30 skin biopsies were generated using RNAseq. Sequencing libraries were constructed using NEBNext Ultra RNA Library Prep Kit, and the RNAseq data were generated with 60 million paired end reads (2×150 bp, Novogene Corporation Inc.)

Quantification of gene expression in each sample is determined as Transcripts Per Million reads (TPM) using RNA-Seq pipeline in OmicSoft Studio (QIAGEN), by choosing Genome Reference Consortium Human Build 38 (GRCh38) for alignment and OmicsoftGenCode.V33 as the gene model (60,699 genes in total).

Log 2 transformation was applied on TPM (after flooring at 0.1), and differential gene expression between two conditions being compared was evaluated using generalized linear model (GLM) on log 2 transformed TPM, followed by multiple comparison correction using false discovery rate (FDR).

Among the 60,699 genes evaluated, 2050 genes were induced after 24-hour culture (vs control) with geometric average of fold change>2 among the 10 donors, and FDR<0.05; 1287 of these 2050 genes were induced (fold change>1.5) in each of the 10 donors.

The % inhibition by IL1α antibody on induced expression of the 1287 injury-induced genes were further calculated in each donor. 139 of the 1287 induced genes had the average of inhibition (among the 10 donors)>20%, and were defined as IL1αRNAseq signature (see FIG. 61. The 139 genes identified are listed in the table below.

| | | Injury | % Inhibi- | |
|---|---|---|---|---|
| | | Fold | tion by | SEQ |
| GeneID | GeneName | Change | antiIL1a | ID NO: |
| ENSG00000164400.6 | CSF2 | 16 | 75 | 19 |
| ENSG00000181617.6 | FDCSP | 84 | 73 | 20 |
| ENSG00000108342.12 | CSF3 | 2635 | 65 | 21 |
| ENSG00000277632.2 | CCL3 | 253 | 62 | 22 |
| ENSG00000163661.4 | PTX3 | 147 | 60 | 23 |
| ENSG00000276085.1 | CCL3L3 | 175 | 59 | 24 |
| ENSG00000125538.12 | IL1B | 45 | 59 | 25 |
| ENSG00000275302.2 | CCL4 | 45 | 58 | 26 |
| ENSG00000169429.11 | CXCL8 | 4065 | 53 | 27 |
| ENSG00000163734.4 | CXCL3 | 990 | 53 | 28 |
| ENSG00000138685.15 | FGF2 | 14 | 53 | 29 |
| ENSG00000163739.5 | CXCL1 | 1114 | 53 | 30 |
| ENSG00000163735.7 | CXCL5 | 957 | 52 | 31 |
| ENSG00000081041.9 | CXCL2 | 800 | 51 | 32 |
| ENSG00000104722.14 | NEFM | 13 | 50 | 33 |
| ENSG00000243649.9 | CFB | 9 | 50 | 34 |
| ENSG00000050730.16 | TNIP3 | 6 | 50 | 35 |
| ENSG00000115009.13 | CCL20 | 39 | 49 | 36 |
| ENSG00000166670.10 | MMP10 | 1478 | 49 | 37 |
| ENSG00000128271.22 | ADORA2A | 6 | 48 | 38 |
| ENSG00000171711.3 | DEFB4A | 172 | 47 | 39 |
| ENSG00000139572.4 | GPR84 | 17 | 46 | 40 |
| ENSG00000277089.4 | AC243829.4 | 6 | 46 | 41 |
| ENSG00000206561.13 | COLQ | 11 | 46 | 42 |
| ENSG00000105825.14 | TFPI2 | 107 | 44 | 43 |
| ENSG00000241794.2 | SPRR2A | 38 | 43 | 44 |
| ENSG00000180914.10 | OXTR | 5 | 43 | 45 |
| ENSG00000149968.12 | MMP3 | 2525 | 43 | 46 |
| ENSG00000276070.5 | CCL4L2 | 26 | 43 | 47 |
| ENSG00000073756.12 | PTGS2 | 533 | 42 | 48 |
| ENSG00000196611.5 | MMP1 | 22185 | 42 | 49 |
| ENSG00000123610.5 | TNFAIP6 | 105 | 42 | 50 |
| ENSG00000188620.11 | HMX3 | 9 | 42 | 51 |
| ENSG00000128342.5 | LIF | 81 | 42 | 52 |
| ENSG00000120217.14 | CD274 | 12 | 42 | 53 |
| ENSG00000279581.1 | AC073862.2 | 8 | 42 | 54 |
| ENSG00000136244.12 | IL6 | 10249 | 40 | 55 |
| ENSG00000164761.9 | TNFRSF11B | 13 | 39 | 56 |
| ENSG00000236453.5 | AC003092.1 | 9 | 39 | 57 |
| ENSG00000103449.12 | SALL1 | 5 | 38 | 58 |
| ENSG00000286256.2 | AC084871.4 | 3 | 38 | 59 |
| ENSG00000004468.13 | CD38 | 4 | 38 | 60 |
| ENSG00000144802.11 | NFKBIZ | 4 | 37 | 61 |
| ENSG00000163874.11 | ZC3H12A | 5 | 37 | 62 |
| ENSG00000080493.17 | SLC4A4 | 8 | 37 | 63 |
| ENSG00000108700.5 | CCL8 | 33 | 36 | 64 |
| ENSG00000272841.1 | AL139393.3 | 8 | 36 | 65 |
| ENSG00000178860.8 | MSC | 5 | 36 | 66 |
| ENSG00000110436.13 | SLC1A2 | 6 | 36 | 67 |
| ENSG00000108691.9 | CCL2 | 19 | 35 | 68 |
| ENSG00000123689.6 | G0S2 | 69 | 35 | 69 |
| ENSG00000157765.13 | SLC34A2 | 8 | 35 | 70 |
| ENSG00000257605.2 | AC073611.1 | 7 | 34 | 71 |
| ENSG00000250771.2 | AC106865.1 | 5 | 34 | 72 |
| ENSG00000237927.1 | AL078604.2 | 11 | 34 | 73 |
| ENSG00000122641.11 | INHBA | 30 | 34 | 74 |
| ENSG00000148346.12 | LCN2 | 5 | 34 | 75 |
| ENSG00000279227.1 | AC009303.4 | 3 | 33 | 76 |
| ENSG00000151790.9 | TDO2 | 9 | 33 | 77 |
| ENSG00000134259.4 | NGF | 19 | 33 | 78 |
| ENSG00000137331.12 | IER3 | 21 | 32 | 79 |
| ENSG00000140379.8 | BCL2A1 | 8 | 32 | 80 |
| ENSG00000112096.18 | SOD2 | 32 | 32 | 81 |
| ENSG00000103888.17 | CEMIP | 239 | 32 | 82 |
| ENSG00000173432.12 | SAA1 | 13 | 32 | 83 |
| ENSG00000056558.11 | TRAF1 | 4 | 32 | 84 |
| ENSG00000131979.19 | GCH1 | 4 | 31 | 85 |
| ENSG00000267009.6 | AC007780.1 | 13 | 31 | 86 |
| ENSG00000267583.5 | AC007998.3 | 6 | 31 | 87 |
| ENSG00000165091.17 | TMC1 | 3 | 31 | 88 |
| ENSG00000152822.14 | GRM1 | 5 | 31 | 89 |
| ENSG00000178172.7 | SPINK6 | 17 | 30 | 90 |
| ENSG00000158473.7 | CD1D | 8 | 30 | 91 |
| ENSG00000124391.5 | IL17C | 8 | 30 | 92 |
| ENSG00000268812.3 | AC004264.1 | 13 | 29 | 93 |
| ENSG00000136160.17 | EDNRB | 6 | 28 | 94 |
| ENSG00000136048.14 | DRAM1 | 8 | 28 | 95 |
| ENSG00000138135.7 | CH25H | 11 | 28 | 96 |
| ENSG00000184492.6 | FOXD4L1 | 4 | 28 | 97 |
| ENSG00000148344.11 | PTGES | 14 | 28 | 98 |
| ENSG00000134339.8 | SAA2 | 132 | 27 | 99 |
| ENSG00000104635.14 | SLC39A14 | 10 | 27 | 100 |
| ENSG00000104312.8 | RIPK2 | 3 | 27 | 101 |
| ENSG00000073150.14 | PANX2 | 4 | 27 | 102 |
| ENSG00000125430.9 | HS3ST3B1 | 4 | 26 | 103 |
| ENSG00000110944.9 | IL23A | 11 | 26 | 104 |
| ENSG00000223949.7 | ROR1-AS1 | 4 | 26 | 105 |
| ENSG00000250519.7 | AP002784.1 | 7 | 26 | 106 |
| ENSG00000006118.14 | TMEM132A | 5 | 26 | 107 |
| ENSG00000154736.6 | ADAMTS5 | 7 | 26 | 108 |
| ENSG00000099985.4 | OSM | 24 | 26 | 109 |
| ENSG00000138835.22 | RGS3 | 3 | 26 | 110 |
| ENSG00000153162.9 | BMP6 | 5 | 26 | 111 |
| ENSG00000159167.12 | STC1 | 389 | 26 | 112 |
| ENSG00000198053.11 | SIRPA | 3 | 25 | 113 |
| ENSG00000196460.14 | RFX8 | 8 | 25 | 114 |
| ENSG00000249992.2 | TMEM158 | 3 | 25 | 115 |
| ENSG00000125813.13 | PAX1 | 10 | 25 | 116 |
| ENSG00000162892.16 | IL24 | 201 | 25 | 117 |
| ENSG00000109205.16 | ODAM | 10 | 25 | 118 |
| ENSG00000060982.15 | BCAT1 | 3 | 25 | 119 |
| ENSG00000279415.1 | AC099494.2 | 4 | 25 | 120 |
| ENSG00000143067.5 | ZNF697 | 3 | 25 | 121 |
| ENSG00000198535.5 | C2CD4A | 82 | 25 | 122 |
| ENSG00000121797.10 | CCRL2 | 6 | 25 | 123 |
| ENSG00000101680.15 | LAMA1 | 18 | 24 | 124 |
| ENSG00000160285.15 | LSS | 2 | 24 | 125 |
| ENSG00000119699.7 | TGFB3 | 4 | 24 | 126 |
| ENSG00000169908.12 | TM4SF1 | 7 | 24 | 127 |
| ENSG00000108688.11 | CCL7 | 81 | 24 | 128 |
| ENSG00000185897.7 | FFAR3 | 7 | 24 | 129 |
| ENSG00000011478.12 | QPCTL | 3 | 24 | 130 |
| ENSG00000089351.14 | GRAMD1A | 4 | 23 | 131 |
| ENSG00000137094.14 | DNAJB5 | 3 | 23 | 132 |
| ENSG00000141337.12 | ARSG | 3 | 23 | 133 |
| ENSG00000118257.16 | NRP2 | 6 | 23 | 134 |
| ENSG00000173404.5 | INSM1 | 3 | 22 | 135 |
| ENSG00000090339.9 | ICAM1 | 6 | 22 | 136 |
| ENSG00000102962.5 | CCL22 | 9 | 22 | 137 |
| ENSG00000174236.4 | REP15 | 6 | 22 | 138 |
| ENSG00000068366.20 | ACSL4 | 4 | 22 | 139 |
| ENSG00000280200.1 | AC073862.5 | 11 | 22 | 140 |
| ENSG00000254087.8 | LYN | 2 | 22 | 141 |
| ENSG00000187134.14 | AKR1C1 | 10 | 22 | 142 |
| ENSG00000146555.19 | SDK1 | 3 | 21 | 143 |
| ENSG00000186340.15 | THBS2 | 6 | 21 | 144 |
| ENSG00000104951.16 | IL4I1 | 14 | 21 | 145 |
| ENSG00000085514.16 | PILRA | 3 | 21 | 146 |
| ENSG00000138821.13 | SLC39A8 | 4 | 21 | 147 |
| ENSG00000204099.11 | NEU4 | 14 | 21 | 148 |
| ENSG00000205362.11 | MT1A | 12 | 21 | 149 |
| ENSG00000175352.11 | NRIP3 | 5 | 21 | 150 |
| ENSG00000205502.4 | C2CD4B | 19 | 20 | 151 |
| ENSG00000174939.11 | ASPHD1 | 4 | 20 | 152 |
| ENSG00000018280.17 | SLC11A1 | 14 | 20 | 153 |
| ENSG00000196805.7 | SPRR2B | 36 | 20 | 154 |
| ENSG00000077150.20 | NFKB2 | 4 | 20 | 155 |
| ENSG00000049249.8 | TNFRSF9 | 4 | 20 | 156 |
| ENSG00000167034.10 | NKX3-1 | 9 | 20 | 157 |

### 6.6. Example 6—Bermekimab Reduces Elevated Level of a Subset of Induced Cytokines in Ex Vivo Biopsy Explant Culture Model at 24-Hour Time Point

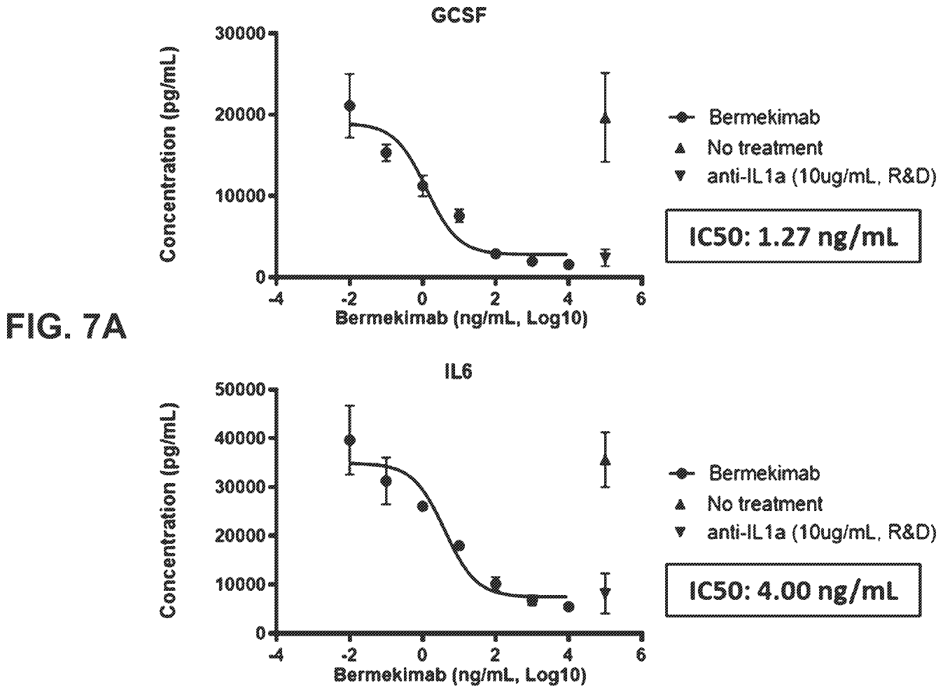

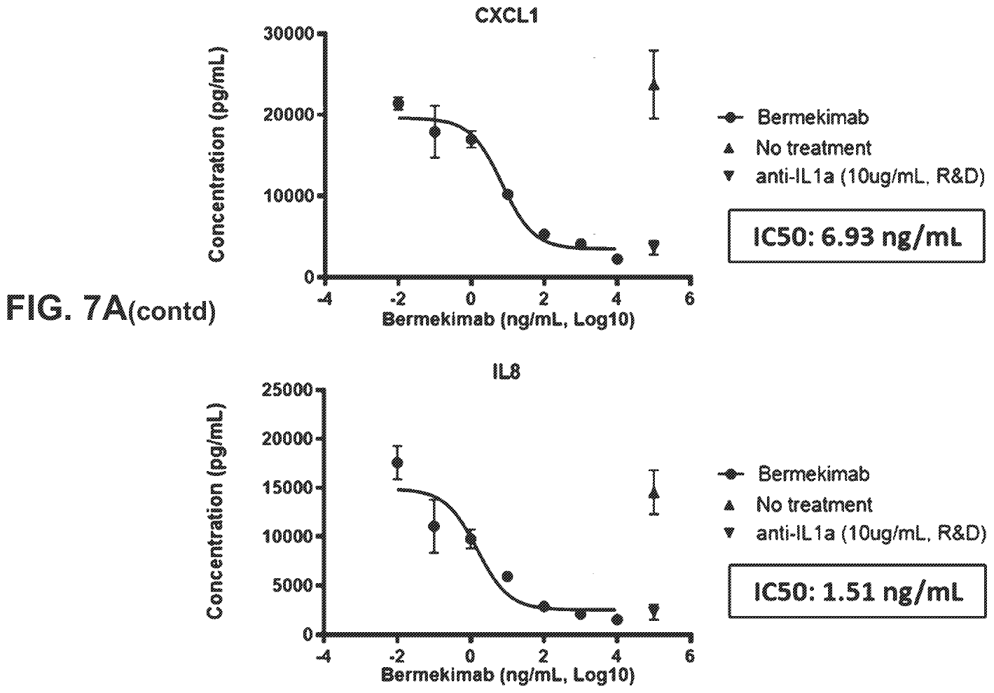

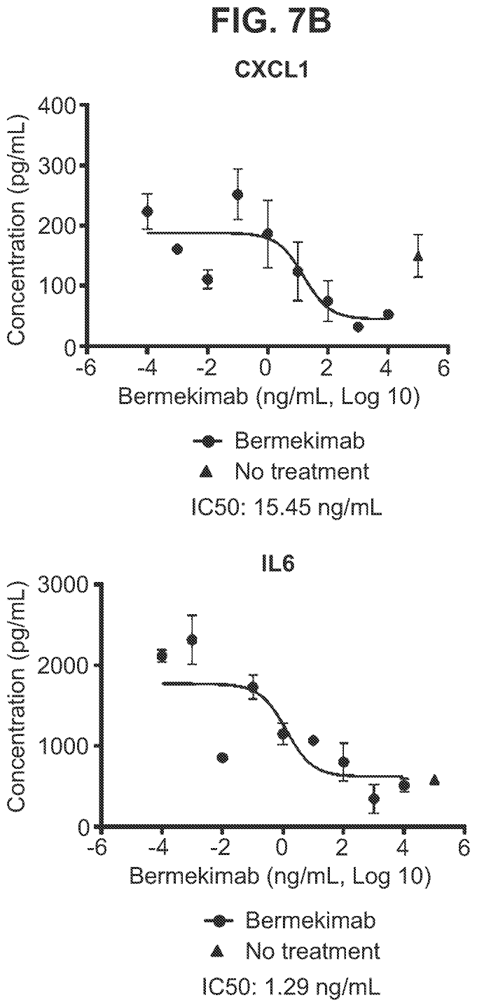

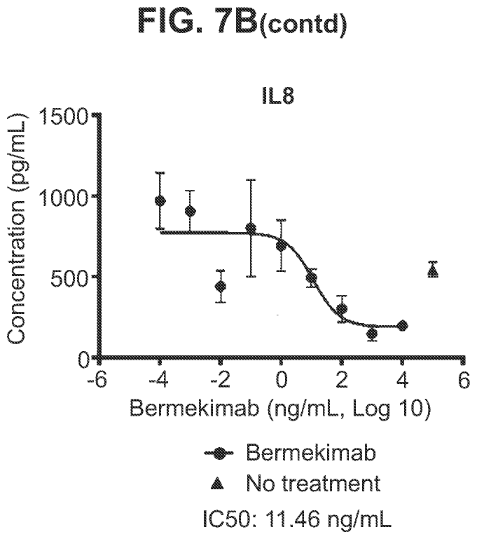

Bermekimab was evaluated in the ex vivo biopsy explant culture assay in 3 healthy skin donors. Bermekimab was tested at various doses (10000, 1000, 100, 10, 1, 0.1 and 0.01 ng/mL; no lidocaine injection was used in this experiment). Commercially available anti-IL1α from R&D (clone 4414, 10 ug/mL) that was utilized to establish the assay and in the studies described above was also included in this experiment. Culture supernatant were collected for cytokines measurement using Luminex while the skin biopsies were used for gene expression analysis in Nanostring. Measurement of cytokine level in the supernatant showed that bermekimab can reduce the level of GCSF, CXCL1, IL6 and IL8 in a dose responsive manner. IC50 values were obtained from the cytokine level measured (see FIG. 7A), and the corresponding IC50 values were calculated. While bermekimab can also reduce the level of IL6, IC50 values can be calculated only in 1 out of the 3 donors evaluated. IC50 values from 3 healthy donors are summarized in the table below.

| IC50 (ng/mL) | Donor 1 | Donor 2 | Donor 3 |
|---|---|---|---|
| GCSF | 1.27 | 5.86 | 0.29 |
| CXCL1 | 6.93 | 20.58 | 28.41 |
| IL6 | 4.01 | — | — |
| IL8 | 1.51 | 10.82 | 2.25 |

Cultured skin biopsies were also collected for further analysis. Skin tissues were processed to generate lysate and the level of cytokines were analyzed similarly using Luminex. Similar to observations in the supernatant, bermekimab can reduce the level of CXCL1, IL6 and IL8 in the skin tissue lysate and the corresponding IC50 values can then be calculated (see FIG. 7B). Unlike in the supernatant, the level of GCSF was not measurable in the tissue lysate.

### 6.7. Example 7—a Phase 1 Study to Investigate the Pharmacokinetics and Pharmacodynamics of Bermekimab in Healthy Participants

This is an open-label, interventional study in healthy participants. Single doses of bermekimab are administered. Three doses of anakinra, the positive PD control, are administered.

There are single-dose SC cohorts and 3 single-dose IV cohorts. Participants are enrolled into either the SC cohorts (400 mg dose at concentrations of 100, 150, 175, and 200 mg/mL; 200 and 800 mg dose at concentration 175 mg/mL) or the IV cohorts (400, 800, and 1,200 mg at 100 mg/mL). There is also a cohort of that receives a 100 mg SC dose of anakinra daily for 3 days to be used as a PD comparator.

The total duration of participation is approximately 16 weeks, including a screening visit up to 28 days prior to study intervention administration. Participants have an inpatient period consisting of 9 days/8 nights. Participants return to the study site at Weeks 2, 3, 4, 6, 8, and 12.

Description of Interventions

Description of Interventions

| Intervention Name | bermekimab | Anakinra |
|---|---|---|
| Dose Formulation | Liquid formulation; | Anakinra is supplied in single-use |
| | bermekimab; Trehalose | preservative free, prefilled glass |
| | Dihydrate; Sodium Phosphate | syringes with 29-gauge needles. |
| | Dibasic; Citric Acid | Each prefilled glass syringe contains |
| | Monohydrate, Phosphoric Acid; | 100 mg of anakinra per 0.67 mL. |
| | Sodium Hydroxide, Water for | The full syringe contains 100 mg |
| | Injection | anakinra. |
| | | Citric acid, anhydrous; Sodium |
| | | chloride; Di sodium edetate |
| | | dihydrate; Polysorbate 80; Sodium |
| | | hydroxide; Water for injections |
| Unit Dose | Subcutaneous (SC): 100 mg/mL, | Injection: 100 mg/0.67 mL solution |
| Strength(s) | 150 mg/mL, 175 mg/mL, | in a single-use prefilled syringe for |
| | 200 mg/mL intravenous (IV): | SC injection. Graduated syringe |
| | 100 mg/mL | allows for doses between 20 and |
| | | 100 mg. |
| Dosage Level(s) | SC: 400 mg at 100 mg/mL, 400 | 100 mg |
| | mg at 150 mg/mL, 400 mg at | |
| | 175 mg/mL, 400 mg at | |
| | 200 mg/mL, 200 mg at | |
| | 175 mg/mL, 800 mg at 175 | |
| | mg/mL | |
| | IV: 400, 800, 1,200 mg | |
| Route of | SC injection and IV infusion | SC injection |
| Administration | | |

Objectives and Endpoints

| | |
|---|---|
| Objectives | Assessments |

Primary

| | |
|---|---|
| To assess the pharmacokinetics (PK) of | PK parameters of bermekimab, including but |
| bermekimab after single subcutaneous (SC) or | not limited to Cₘₐₓ, AUC_{inf}, AUCₗₐₛₜ, T_{1/2} and |
| intravenous (IV) administrations and the effect | F(%) |
| of formulation concentrations on PK of | |
| bermekimab in healthy participants. | |

Secondary

| | |
|---|---|
| To evaluate safety, tolerability, and | Proportion of participants with treatment- |
| immunogenicity of a single SC or IV | emergent adverse events (TEAE) by severity |
| administration of bermekimab in healthy | and serious adverse events (SAE) through |
| participants. | Day 85. |
| | Clinically significant changes in vital signs, |
| | electrocardiograms (ECGs), hematology, |
| | chemistry, and urinalysis. |
| | Presence of antibodies to bermekimab. |

Exploratory

| | |
|---|---|
| To assess pharmacodynamic (PD) modulation | Biopsy, incubation, and molecular endpoint |
| of interleukin-1 alpha (IL-1α) in comparison to | assessment in skin biopsies and secreted |
| a known antagonist of IL-1α and β by | cytokines before and after treatment with |
| provoking the release of IL-1α in healthy skin | bermekimab or anakinra. |
| and assessing the effect of blockade on | |
| downstream PD readouts. | |

This study employs a 2-wave dosing scheme. Wave 1 consists of Cohorts A through E and Wave 2 consists of Cohorts F through J. The cohorts in Wave 1 are randomized and dosed in a parallel manner according to site logistics. The cohorts in Wave 2 are not randomized but are enrolled in sequential order, ie, Cohort F is fully enrolled before starting Cohort G enrollment and so on.

| Wave 1 (Randomized | Wave 2 (Non-Randomized |
|---|---|
| Parallel Cohorts) | Sequential Cohorts) |
| Cohort A - 400 mg SC | Cohort F - 200 mg SC |
| (formulation: 100 mg/mL) | (formulation: 175 mg/mL) |
| Cohort B - 400 mg SC | Cohort G - 800 mg SC |
| (formulation: 150 mg/mL) | (formulation: 175 mg/mL) |
| Cohort C - 400 mg SC | Cohort H - 800 mg IV |
| (formulation: 175 mg/mL) | (formulation: 100 mg/mL) |
| Cohort D - 400 mg SC | Cohort I - 1,200 mg IV |
| (formulation: 200 mg/mL) | (formulation: 100 mg/mL) |
| Cohort E - 400 mg IV | Cohort J - 100 mg SC |
| (formulation: 100 mg/mL) | anakinra |

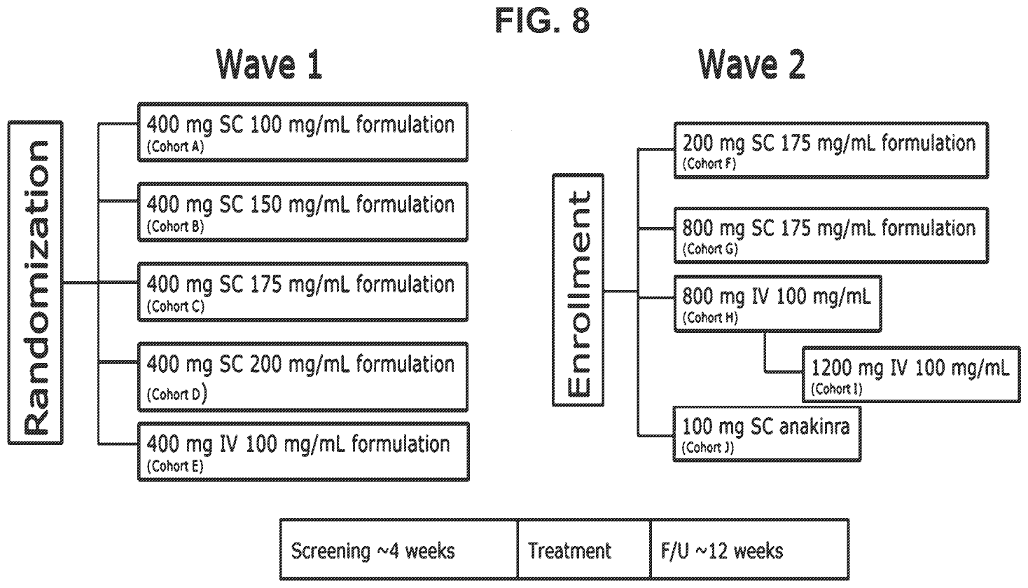

FIG. 8 is a schematic summarizing the design of the Phase 1 study.

### Pharmacokinetics

Serum and plasma samples are analyzed to determine concentrations of bermekimab using a validated, specific, and sensitive immunoassay method.

Pharmacokinetic parameters of bermekimab are calculated from concentrations over time data using noncompartmental analyses. Pharmacokinetic parameters following a single IV or SC administration of bermekimab include, but are not limited to:

IV only:
Cₘₐₓ: maximum observed plasma concentration.
AUCinf: area under the plasma concentration versus time curve from time zero to infinity with extrapolation of the terminal phase.
AUCₗₐₛₜ: area under the plasma concentration versus time curve from time zero to the time corresponding to the last quantifiable concentration.
T_{1/2}: terminal half-life.
CL: total systemic clearance.
V_{z}: volume of distribution based on terminal phase.
   SC only:
Cₘₐₓ: maximum observed plasma concentration.
Tₘₐₓ: time to reach maximum observed plasma concentration.
AUC_{inf}: area under the plasma concentration versus time curve from time zero to infinity with extrapolation of the terminal phase.
AUCₗₐₛₜ: area under the plasma concentration versus time curve from time zero to the time corresponding to the last quantifiable concentration.
T_{1/2}: terminal half-life.
CL/F: apparent total systemic clearance after extravascular administration.
V_{z}/F: apparent volume of distribution based on terminal phase after extravascular administration.
F (%): absolute SC bioavailability to be calculated using the following equation.

$$F ( \% ) = \frac{A U {C}_{inf , SC}}{mean {AUC}_{inf , IV}} \times 100 \%$$

### Pharmacodynamics

Participants are required to have 4, 4-mm skin punch biopsies of normal healthy skin (2 pre- and 2 posttreatment). Assessing the pattern of gene expression and protein production in the skin biopsies allows measurement of PD effect of bermekimab or anakinra (which serves as a positive control) on molecular events that have an established dependence on IL-1α. At each skin biopsy collection visit, one skin biopsy is processed immediately as per skin biopsy lab manual and the second skin biopsy is cultured ex vivo for 24 hours to establish the stimulation baseline for each participant (the stimulus is the skin biopsy procedure itself). The third and fourth skin biopsies are collected after bermekimab administration and 1 skin biopsy specimen is processed immediately as per skin biopsy laboratory manual and the second skin biopsy specimen is cultured ex vivo for 24 hours. The effects of bermekimab or anakinra dosing on gene and protein expression patterns induced as a result of the tissue injury from the skin biopsy collection procedure are determined by comparing changes relative to baseline in predefined gene expression signatures and secreted proteins in the supernatants. Skin biopsy specimens are assessed for gene expression and for secreted proteins accumulation in ex vivo culture supernatants.

Data from skin biopsy samples is analyzed to evaluate PD effects of each intervention on the induction of gene expression changes and secreted protein levels in response to the wounding process (biopsy procedure). Pharmacodynamic data is presented using suitable descriptive statistics for each intervention, comparing PD readouts in ex vivo-cultured pretreatment biopsies versus ex vivo-cultured posttreatment biopsies (biopsy samples that are not cultured ex vivo serve as control for changes occurring during ex vivo incubation). For each PD parameter (e.g., expression levels of select genes, concentrations of protein readouts; % inhibition), summary plots (e.g., mean and standard deviation or median and interquartile ranges) of absolute levels and changes from pretreatment are generated for each cohort.

Thus, these studies identified a set of biomarkers (e.g., secreted cytokines and gene signature) to monitor and assess effects (e.g., pharmacodynamics and/or pharmacokinetic effects) of an IL1α inhibitor among other uses.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of identifying a subject who is likely to be responsive to a treatment comprising an inhibitor of IL1α, or predicting the responsiveness of a subject to a treatment comprising an inhibitor of IL1α, comprising:
i. providing a sample from the subject;
ii. administering the inhibitor of IL1α to the sample;
iii. measuring levels of one or more biomarkers in the sample;
iv. identifying the subject as being likely to be responsive to the treatment comprising the inhibitor of IL1α, or predicting the responsiveness of the subject to the treatment comprising the inhibitor of IL1α, based on the levels of the one or more biomarkers as measured in step (iii).

2. A method of selectively treating a subject with a treatment comprising an inhibitor of IL1α, comprising administering a therapeutically effective amount of the treatment comprising the inhibitor of IL1α to the subject identified as being likely to be responsive to the treatment comprising the inhibitor of IL1α according to the method of claim 1.

3. The method of claim 1 or claim 2, wherein the sample comprises a skin cell.

4. The method of any one of claims 1 to 3, wherein the sample comprises an injured skin cell.

5. The method of any one of claims 1 to 3, wherein the sample is obtained by a skin biopsy procedure.

6. The method of claim 5, wherein the size of the sample is about 3.5 mm to 4.5 mm, wherein optionally the size of the sample is about 4.0 mm.

7. The method of any one of claims 1 to 6 further comprising culturing the sample ex vivo prior to administering the inhibitor of IL1α to the sample.

8. The method of any one of claims 1 to 6 further comprising culturing the sample ex vivo after administering the inhibitor of IL1α to the sample.

9. The method of any one of claims 1 to 6, wherein the inhibitor of IL1α is administered to the sample while culturing the sample ex vivo.

10. The method of any one of claims 1 to 9, wherein the levels of the one or more biomarkers are measured at least 4 hours, at least 5 hours, at least 10 hours, at least 15 hours, at least 20 hours, at least 21 hours, at least 22 hours, at least 23 hours, or at least 24 hours post the administration of the inhibitor of IL1α and/or post the sample is obtained from the subject.

11. The method of any one of claims 1 to 9, wherein the level of the one or more biomarkers are measured at about 24 hours or at least 24 hours post the administration of the inhibitor of IL1α and/or post the sample is obtained from the subject.

12. The method of any one of claims 1 to 11, further comprising comparing the levels of the one or more biomarkers with reference levels of the one or more biomarkers.

13. The method of claim 12, wherein the reference levels of the one or more biomarkers are the levels of the one or more biomarkers in a reference sample from the subject prior to administration of the inhibitor of IL1α.

14. The method of claim 12, wherein the reference levels of the one or more biomarkers are the levels of the one or more biomarkers in a reference sample from the subject without administration of the inhibitor of IL1α.

15. The method of claim 12, wherein the reference levels of the one or more biomarkers are pre-determined levels of the one or more biomarkers.

16. The method of claim 12, wherein the reference levels of the one or more biomarkers are the levels of the one or more biomarkers in a reference sample administered with a control agent.

17. The method of claim 16, wherein the control agent is a positive control agent that inhibits IL1α, for example the anti-IL1α antibody from R&D Systems (clone 4414).

18. The method of claim 16, wherein the control agent is a negative control agent that does not inhibit IL1α.

19. The method of any one of claims 13 to 16 and 18, wherein the lower levels of the one or more biomarkers as compared with reference levels of the one or more biomarkers indicates the subject is likely to be responsive to the treatment comprising the inhibitor of IL1α, or wherein the subject is identified likely to be responsive to the treatment comprising the inhibitor of IL1α if the levels of the one or more biomarkers in the sample are at least 20%, at least 30%, at least 35%, at least 40%, or at least 50% less than the reference levels.

20. The method of any one of claims 1 to 19, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

21. The method of any one of claims 1 to 19, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2.

22. The method of any one of claims 1 to 19, wherein the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

23. The method of claim 20, wherein the one or more biomarkers comprises CCL20.

24. The method of claim 20, wherein the one or more biomarkers comprises CCL22.

25. The method of claim 20, wherein the one or more biomarkers comprises CSF3, wherein optionally the one or more biomarkers comprise CSF3 (GCSF), CXCL1 and IL6.

26. The method of claim 20, wherein the one or more biomarkers comprises CXCL1.

27. The method of claim 20, wherein the one or more biomarkers comprises CXCL2.

28. The method of claim 20, wherein the one or more biomarkers comprises CXCL3.

29. The method of claim 20, wherein the one or more biomarkers comprises CXCL5.

30. The method of claim 20, wherein the one or more biomarkers comprises CXCL6.

31. The method of claim 20, wherein the one or more biomarkers comprises IL6.

32. The method of claim 20, wherein the one or more biomarkers comprises IL8.

33. The method of claim 20, wherein the one or more biomarkers comprises PTGS2.

34. The method of claim 20, wherein the one or more biomarkers comprises CCL3.

35. The method of claim 20, wherein the one or more biomarkers comprises CCL4.

36. The method of claim 20, wherein the one or more biomarkers comprises CCL8.

37. The method of claim 20, wherein the one or more biomarkers comprises CFB.

38. The method of claim 20, wherein the one or more biomarkers comprises IL1B.

39. The method of claim 20, wherein the one or more biomarkers comprises MMP3.

40. The method of claim 20, wherein the one or more biomarkers are CCL20, CCL22, CSF3(GCSF), CXCL1, CXCL2, CXCL3, CXCL5, CXCL6, IL6, IL8, and PTGS2.

41. The method of claim 40, wherein a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

42. The method of claim 20, wherein the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2.

43. The method of claim 42, wherein a composite score is calculated based on the levels of CCL20, CCL3, CCL4, CCL8, CFB, CSF3(GMCSF), CXCL1, CXCL2, CXCL3, CXCL5, IL1B, IL6, IL8, MMP3, and PTGS2, and wherein the method further comprises comparing the composite score to a reference score.

44. The method of claim 20, wherein the one or more biomarkers are CCL20, CSF3(GCSF), CXCL1, CXCL2, CXCL3, CXCL5, IL6, IL8, and PTGS2.

45. The method of claim 44, wherein a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

46. The method of any one of claims 20 to 45, wherein the levels of the one or more biomarkers are determined by measuring the nucleic acid levels of the one or more biomarkers, wherein optionally the nucleic acid is mRNA.

47. The method of any one of claims 20 to 45, wherein the levels of the one or more biomarkers are determined by measuring the protein levels of the one or more biomarkers.

48. The method of any one of claims 1 to 19, wherein the levels of the one or more biomarkers are the levels of cytokines secreted by the sample selected from a group consisting of GCSF, CXCL1, IL4, IL6, IL8, MDC, IP10, GMCSF, MIP1a and TGFa.

49. The method of any one of claims 1 to 19, wherein the levels of the one or more biomarkers are the levels of cytokines secreted by the sample selected from a group consisting of GCSF, CXCL1, IL4, IL6, IL8, MDC and IP10.

50. The method of any one of claims 1 to 19, wherein the levels of the one or more biomarkers are the levels of cytokines secreted by the sample selected from a group consisting of CXCL1, GCSF, GMCSF, IL6, IL8, MIP1a and TGFa.

51. The method of claim 48, wherein the one or more biomarkers comprises GCSF, wherein optionally the one or more biomarkers comprise GCSF, CXCL1 and IL6.

52. The method of claim 48, wherein the one or more biomarkers comprises CXCL1.

53. The method of claim 48, wherein the one or more biomarkers comprises IL4.

54. The method of claim 48, wherein the one or more biomarkers comprises IL6.

55. The method of claim 48, wherein the one or more biomarkers comprises IL8.

56. The method of claim 48, wherein the one or more biomarkers comprises MDC.

57. The method of claim 48, wherein the one or more biomarkers comprises IP10.

58. The method of claim 48, wherein the one or more biomarkers comprises GMCSF.

59. The method of claim 48, wherein the one or more biomarkers comprises MIP1a.

60. The method of claim 48, wherein the one or more biomarkers comprises TGFa.

61. The method of claim 48, wherein the one or more biomarkers are GCSF, CXCL1, IL4, IL6, IL8, MDC and IP10.

62. The method of claim 61, wherein a composite score is calculated based on the levels of GCSF, CXCL1, IL4, IL6, IL8, MDC and IP10, and wherein the method further comprises comparing the composite score to a reference score.

63. The method of claim 48, wherein the one or more biomarkers are CXCL1, GCSF, GMCSF, IL6, IL8, MIP1a and TGFa.

64. The method of claim 63, wherein a composite score is calculated based on the levels of CXCL1, GCSF, GMCSF, IL6, IL8, MIP1a and TGFa, and wherein the method further comprises comparing the composite score to a reference score.

65. The method of claim 48, wherein the one or more biomarkers are GCSF, CXCL1, IL6 and IL8.

66. The method of claim 65, wherein a composite score is calculated based on the levels of GCSF, CXCL1, IL6 and IL8, and wherein the method further comprises comparing the composite score to a reference score.

67. The method of any one of claims 48 to 66, wherein the levels of the one or more biomarkers are determined by measuring the protein levels of the one or more biomarkers.

68. A method of identifying a subject having a IL1α mediated disease who is likely to be responsive to a treatment comprising an inhibitor of IL1α or predicting the responsiveness of a subject having a IL1α mediated disease to a treatment comprising an inhibitor of IL1α, comprising:
i. providing a sample from the subject;
ii. measuring levels of one or more biomarkers in the sample;
iii. identifying the subject with the IL1α mediated disease as responsive to the treatment with the inhibitor of IL1α when the levels of one or more biomarkers in the sample are higher than the reference levels of one or more biomarkers; and/or identifying the subject with the IL1α mediated disease as non-responsive to the treatment with the inhibitor of IL1α when the levels of one or more biomarkers in the sample are not higher than the reference levels of one or more biomarkers.

69. A method of selectively treating a subject having a IL1α mediated disease with a treatment comprising an inhibitor of IL1α, comprising administering a therapeutically effective amount of the treatment comprising the inhibitor of IL1α to the subject identified responsive to the treatment comprising the inhibitor of IL1α according to the method of claim 68.

70. The method of claim 68 or 69, wherein the sample comprises a skin cell, and wherein optionally the skin cell is an injured skin cell.

71. The method of claim 68 or 69, wherein the sample is obtained by a skin biopsy procedure.

72. The method of claim 68 or 69, wherein the sample is a blood sample.

73. The method of any one of claims 68 to 72, wherein the reference levels of the one or more biomarkers are pre-determined levels of the one or more biomarkers.

74. The method of any one of claims 68 to 72, wherein the reference level of the one or more biomarkers are the levels of the one or more biomarkers in a sample comprising an uninjured skin cell.

75. The method of any one of claims 68 to 74, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

76. The method of any one of claims 68 to 74, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2.

77. The method of any one of claims 68 to 74, wherein the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

78. The method of claim 75, wherein the one or more biomarkers comprises CCL20.

79. The method of claim 75, wherein the one or more biomarkers comprises CCL22.

80. The method of claim 75, wherein the one or more biomarkers comprises CSF3, wherein optionally the one or more biomarkers comprise CSF3 (GCSF), CXCL1 and IL6.

81. The method of claim 75, wherein the one or more biomarkers comprises CXCL1.

82. The method of claim 75, wherein the one or more biomarkers comprises CXCL2.

83. The method of claim 75, wherein the one or more biomarkers comprises CXCL3.

84. The method of claim 75, wherein the one or more biomarkers comprises CXCL5.

85. The method of claim 75, wherein the one or more biomarkers comprises CXCL6.

86. The method of claim 75, wherein the one or more biomarkers comprises IL6.

87. The method of claim 75, wherein the one or more biomarkers comprises IL8.

88. The method of claim 75, wherein the one or more biomarkers comprises PTGS2.

89. The method of claim 75, wherein the one or more biomarkers comprises CCL3.

90. The method of claim 75, wherein the one or more biomarkers comprises CCL4.

91. The method of claim 75, wherein the one or more biomarkers comprises CCL8.

92. The method of claim 75, wherein the one or more biomarkers comprises CFB.

93. The method of claim 75, wherein the one or more biomarkers comprises IL1B.

94. The method of claim 75, wherein the one or more biomarkers comprises MMP3.

95. The method of claim 75, wherein the one or more biomarkers are CCL20, CCL22, CSF3(GCSF), CXCL1, CXCL2, CXCL3, CXCL5, CXCL6, IL6, IL8, and PTGS2.

96. The method of claim 95, wherein a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

97. The method of claim 75, wherein the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2.

98. The method of claim 97, wherein a composite score is calculated based on the levels of CCL20, CCL3, CCL4, CCL8, CFB, CSF3(GCSF), CXCL1, CXCL2, CXCL3, CXCL5, IL1B, IL6, IL8, MMP3, and PTGS2, and wherein the method further comprises comparing the composite score to a reference score.

99. The method of claim 75, wherein the one or more biomarkers are CCL20, CSF3(GCSF), CXCL1, CXCL2, CXCL3, CXCL5, IL6, IL8, and PTGS2.

100. The method of claim 99, wherein a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

101. The method of any one of claims 75 to 100, wherein the levels of the one or more biomarkers are determined by measuring the nucleic acid levels of the one or more biomarkers, wherein optionally the nucleic acid is mRNA.

102. The method of any one of claims 75 to 100, wherein the levels of the one or more biomarkers are determined by measuring the protein levels of the one or more biomarkers.

103. The method of any one of claims 68 to 74, wherein the levels of the one or more biomarkers are the levels of cytokines secreted by the sample selected from a group consisting of GCSF, CXCL1, IL4, IL6, IL8, MDC, IP10, GMCSF, MIP1a and TGFa.

104. The method of any one of claims 68 to 74, wherein the levels of the one or more biomarkers are the levels of cytokines secreted by the sample selected from a group consisting of GCSF, CXCL1, IL4, IL6, IL8, MDC and IP10.

105. The method of any one of claims 68 to 74, wherein the levels of the one or more biomarkers are the levels of cytokines secreted by the sample selected from a group consisting of CXCL1, GCSF, GMCSF, IL6, IL8, MIP1a and TGFa.

106. The method of claim 103, wherein the one or more biomarkers comprises GCSF, wherein optionally the one or more biomarkers comprise GCSF, CXCL1 and IL6.

107. The method of claim 103, wherein the one or more biomarkers comprises CXCL1.

108. The method of claim 103, wherein the one or more biomarkers comprises IL4.

109. The method of claim 103, wherein the one or more biomarkers comprises IL6.

110. The method of claim 103, wherein the one or more biomarkers comprises IL8.

111. The method of claim 103, wherein the one or more biomarkers comprises MDC.

112. The method of claim 103, wherein the one or more biomarkers comprises IP10.

113. The method of claim 103, wherein the one or more biomarkers comprises GMCSF.

114. The method of claim 103, wherein the one or more biomarkers comprises MIP1a.

115. The method of claim 103, wherein the one or more biomarkers comprises TGFa.

116. The method of claim 103, wherein the one or more biomarkers are GCSF, CXCL1, IL4, IL6, IL8, MDC and IP10.

117. The method of claim 116, wherein a composite score is calculated based on the levels of GCSF, CXCL1, IL4, IL6, IL8, MDC and IP10, and wherein the method further comprises comparing the composite score to a reference score.

118. The method of claim 103, wherein the one or more biomarkers are CXCL1, GCSF, GMCSF, IL6, IL8, MIP1a and TGFa.

119. The method of claim 118, wherein a composite score is calculated based on the levels of CXCL1, GCSF, GMCSF, IL6, IL8, MIP1a and TGFa, and wherein the method further comprises comparing the composite score to a reference score.

120. The method of claim 103, wherein the one or more biomarkers are GCSF, CXCL1, IL6 and IL8.

121. The method of claim 120, wherein a composite score is calculated based on the levels of GCSF, CXCL1, IL6 and IL8, and wherein the method further comprises comparing the composite score to a reference score.

122. The method of any one of claims 103 to 121, wherein the levels of the one or more biomarkers are determined by measuring the protein levels of the one or more biomarkers.

123. A method of predicting the responsiveness of a subject having an IL1α mediated disease to an IL1α inhibitor or determining an effective dose of an IL1α inhibitor to be administered to a subject having an IL1α mediated disease comprising:
i. providing a first sample from a subject;
ii. administering an IL1α inhibitor to the subject at a predefined dose;
iii. providing a second sample from the subject;
iv. measuring levels of one or more biomarkers in the first sample and the second sample; and
v. determining that the subject is likely to be responsive to the IL1α inhibitor or the predefined dose is effective when the levels of the one or more biomarkers in the second sample are lower than the levels of one or more biomarkers in the first sample.

124. The method of claim 123, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

125. The method of claim 123, wherein the levels of the one or more biomarkers are the expression levels of one or more biomarkers selected from a group consisting of CCL20, CCL22, CSF3(GCSF), CXCL1, CXCL2, CXCL3, CXCL5, CXCL6, IL6, IL8, and PTGS2.

126. The method of claim 123, wherein the levels of the one or more biomarkers are the expression levels of one or more biomarkers selected from a group consisting of CCL20, CCL3, CCL4, CCL8, CFB, CSF3(GCSF), CXCL1, CXCL2, CXCL3, CXCL5, IL1B, IL6, IL8, MMP3, and PTGS2.

127. The method of claim 124, wherein the one or more biomarkers comprises CCL20.

128. The method of claim 124, wherein the one or more biomarkers comprises CCL22.

129. The method of claim 124, wherein the one or more biomarkers comprises CSF3, wherein optionally the one or more biomarkers comprise CSF3 (GCSF), CXCL1 and IL6.

130. The method of claim 124, wherein the one or more biomarkers comprises CXCL1.

131. The method of claim 124, wherein the one or more biomarkers comprises CXCL2.

132. The method of claim 124, wherein the one or more biomarkers comprises CXCL3.

133. The method of claim 124, wherein the one or more biomarkers comprises CXCL5.

134. The method of claim 124, wherein the one or more biomarkers comprises CXCL6.

135. The method of claim 124, wherein the one or more biomarkers comprises IL6.

136. The method of claim 124, wherein the one or more biomarkers comprises IL8.

137. The method of claim 124, wherein the one or more biomarkers comprises PTGS2.

138. The method of claim 124, wherein the one or more biomarkers comprises CCL3.

139. The method of claim 124, wherein the one or more biomarkers comprises CCL4.

140. The method of claim 124, wherein the one or more biomarkers comprises CCL8.

141. The method of claim 124, wherein the one or more biomarkers comprises CFB.

142. The method of claim 124, wherein the one or more biomarkers comprises IL1B.

143. The method of claim 124, wherein the one or more biomarkers comprises MMP3.

144. The method of claim 124, wherein the biomarkers are CCL20, CCL22, CSF3(GCSF), CXCL1, CXCL2, CXCL3, CXCL5, CXCL6, IL6, IL8, PTGS2, CCL3, CCL4, CCL8, CFB, IL1B, and MMP3.

145. The method of claim 124, wherein the one or more biomarkers are CCL20, CCL22, CSF3(GCSF), CXCL1, CXCL2, CXCL3, CXCL5, CXCL6, IL6, IL8, and PTGS2.

146. The method of claim 145, wherein a composite score is calculated based on the levels of CCL20, CCL22, CSF3, CXCL1, CXCL2, CXCL3, CXCL5, CXCL6, IL6, IL8, and PTGS2, and wherein the method further comprises comparing the composite score to a reference score.

147. The method of claim 124, wherein the one or more biomarkers are CCL20, CCL3, CCL4, CCL8, CFB, CSF3(GCSF), CXCL1, CXCL2, CXCL3, CXCL5, IL1B, IL6, IL8, MMP3, and PTGS2.

148. The method of claim 147, wherein a composite score is calculated based on the levels of CCL20, CCL3, CCL4, CCL8, CFB, CSF3(GMCSF), CXCL1, CXCL2, CXCL3, CXCL5, IL1B, IL6, IL8, MMP3, and PTGS2, and wherein the method further comprises comparing the composite score to a reference score.

149. The method of claim 124, wherein the one or more biomarkers are CCL20, CSF3(GCSF), CXCL1, CXCL2, CXCL3, CXCL5, IL6, IL8, and PTGS2.

150. The method of claim 149, wherein a composite score is calculated based on the levels of CCL20, CSF3(GCSF), CXCL1, CXCL2, CXCL3, CXCL5, IL6, IL8, and PTGS2, and wherein the method further comprises comparing the composite score to a reference score.

151. The method of any one of claims 124 to 150, wherein the levels of the one or more biomarkers are determined by measuring the nucleic acid levels of the one or more biomarkers, wherein optionally the nucleic acid is mRNA.

152. The method of any one of claims 124 to 150, wherein the levels of the one or more biomarkers are determined by measuring the protein levels of the one or more biomarkers.

153. The method of claim 123, wherein the levels of the one or more biomarkers are the levels of cytokines secreted by the sample selected from a group consisting of GCSF, CXCL1, IL4, IL6, IL8, MDC, IP10, GMCSF, MIP1a and TGFa.

154. The method of claim 123, wherein the levels of the one or more biomarkers are the levels of cytokines secreted by the sample selected from a group consisting of GCSF, CXCL1, IL4, IL6, IL8, MDC and IP10.

155. The method of claim 123, wherein the levels of the one or more biomarkers are the levels of cytokines secreted by the sample selected from a group consisting of CXCL1, GCSF, GMCSF, IL6, IL8, MIP1a and TGFa.

156. The method of claim 153, wherein the one or more biomarkers comprises GCSF, wherein optionally the one or more biomarkers comprise GCSF, CXCL1 and IL6.

157. The method of claim 153, wherein the one or more biomarkers comprises CXCL1.

158. The method of claim 153, wherein the one or more biomarkers comprises IL4.

159. The method of claim 153, wherein the one or more biomarkers comprises IL6.

160. The method of claim 103, wherein the one or more biomarkers comprises IL8.

161. The method of claim 153, wherein the one or more biomarkers comprises MDC.

162. The method of claim 153, wherein the one or more biomarkers comprises IP10.

163. The method of claim 153, wherein the one or more biomarkers comprises GMCSF.

164. The method of claim 153, wherein the one or more biomarkers comprises MIP1a.

165. The method of claim 153, wherein the one or more biomarkers comprises TGFa.

166. The method of claim 153, wherein the one or more biomarkers are GCSF, CXCL1, IL4, IL6, IL8, MDC and IP10.

167. The method of claim 166, wherein a composite score is calculated based on the levels of GCSF, CXCL1, IL4, IL6, IL8, MDC and IP10, and wherein the method further comprises comparing the composite score to a reference score.

168. The method of claim 153, wherein the one or more biomarkers are CXCL1, GCSF, GMCSF, IL6, IL8, MIP1a and TGFa.

169. The method of claim 168, wherein a composite score is calculated based on the levels of CXCL1, GCSF, GMCSF, IL6, IL8, MIP1a and TGFa, and wherein the method further comprises comparing the composite score to a reference score.

170. The method of claim 153, wherein the one or more biomarkers are GCSF, CXCL1, IL6 and IL8.

171. The method of claim 170, wherein a composite score is calculated based on the levels of GCSF, CXCL1, IL6 and IL8, and wherein the method further comprises comparing the composite score to a reference score.

172. The method of any one of claims 153 to 171, wherein the levels of the one or more biomarkers are determined by measuring the protein levels of the one or more biomarkers.

173. The method of any one of claim 1-19, 68-74 or 123 wherein the levels of the one or more biomarkers are the expression levels of one or more biomarkers selected from a group consisting of GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

174. The method of claim 173, wherein the biomarkers are GCSF (CSF3), CXCL1, IL6, GMCSF (CSF2), CCL20, CCL22, CXCL2, CXCL3, CXCL5, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

175. The method of any one of claim 1-19, 68-74 or 123 wherein the levels of the one or more biomarkers are the expression levels of one or more biomarkers selected from a group consisting of GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

176. The method of claim 175, wherein the biomarkers are GCSF (CSF3), CXCL1, IL6, IL8, IL4, MDC, IP10, GMCSF (CSF2), MIP1a, TGFa, CCL20, CCL22, CXCL2, CXCL3, CXCL5, CXCL6, IL1B, PTGS2, CCL3, CCL4, CCL8, CFB, MMP3, FDCSP, PTX3, CCL3L3, CXCL8, FGF2, NEFM, TNIP3, MMP10, ADORA2A, DEFB4A, GPR84, AC243829.4, COLQ, TFPI2, SPRR2A, OXTR, CCL4L2, MMP1, TNFAIP6, HMX3, LIF, CD274, AC073862.2, TNFRSF11B, AC003092.1, SALL1, AC084871.4, CD38, NFKBIZ, ZC3H12A, SLC4A4, AL139393.3, MSC, SLC1A2, CCL2, GOS2, SLC34A2, AC073611.1, AC106865.1, AL078604.2, INHBA, LCN2, AC009303.4, TDO2, NGF, IER3, BCL2A1, SOD2, CEMIP, SAA1, TRAF1, GCH1, AC007780.1, AC007998.3, TMC1, GRM1, SPINK6, CD1D, IL17C, AC004264.1, EDNRB, DRAM1, CH25H, FOXD4L1, PTGES, SAA2, SLC39A14, RIPK2, PANX2, HS3ST3B1, IL23A, ROR1-AS1, AP002784.1, TMEM132A, ADAMTS5, OSM, RGS3, BMP6, STC1, SIRPA, RFX8, TMEM158, PAX1, IL24, ODAM, BCAT1, AC099494.2, ZNF697, C2CD4A, CCRL2, LAMA1, LSS, TGFB3, TM4SF1, CCL7, FFAR3, QPCTL, GRAMD1A, DNAJB5, ARSG, NRP2, INSM1, ICAM1, REP15, ACSL4, AC073862.5, LYN, AKR1C1, SDK1, THBS2, IL4I1, PILRA, SLC39A8, NEU4, MT1A, NRIP3, C2CD4B, ASPHD1, SLC11A1, SPRR2B, NFKB2, TNFRSF9, and NKX3-1.

177. The method of any one of claims 68-175, wherein the IL1α mediated disease is atopic dermatitis.

178. The method of any one of claims 68-175, wherein the IL1α mediated disease is hidradenitis suppurativa.

179. The method of any preceding claim, wherein the inhibitor of IL1α is an anti-IL1α antibody.

180. The method of claim 179, wherein the anti-IL1α antibody is bermekimab.

181. A kit for identifying a subject who is likely to be responsive to a treatment comprising an inhibitor of IL1α, predicting the responsiveness of a subject to a treatment comprising an inhibitor of IL1α, or monitoring the response of a subject to a treatment comprising an inhibitor of IL1α, comprising:
(a) an agent for measuring levels of one or more biomarkers in a sample, wherein the one or more biomarkers are selected from (i) a group consisting of CCL20, CCL22, CSF3, (GCSF), CXCL1, CXCL2, CXCL3, CXCL5, CXCL6, IL6, IL8, and PTGS2, CCL3, CCL4, CCL8, CFB, IL1B, and MMP3, or (ii) a group consisting of GCSF, CXCL1, IL4, IL6, IL8, MDC, IP10, GMCSF, MIP1a and IP10TGFa; and
(b) a positive control agent that inhibits IL1α and/or a negative control agent that does not inhibit IL1α.

182. The kit of claim 181, wherein the kit further comprises a tool for obtaining the sample from a subject.

183. The kit of claim 182, wherein the tool is suitable for obtaining a skin sample from the subject.

184. The kit of any one of claims 181 to 183, wherein the kit further comprises a tool for administering the inhibitor of IL1α to the sample.

185. The kit of any one of claims 181 to 184, wherein the positive control agent is an antibody that binds to IL1α.

186. The method or kit of any one of claims 1 to 185, wherein the subject has atopic dermatitis, hidradenitis suppurativa, psoriasis, cutaneous lupus erythematosus, or autoimmune bullous disease.

187. The method or kit of claim 186, wherein the subject has atopic dermatitis.

188. The method or kit of claim 186, wherein the subject has hidradenitis suppurativa.
